(19) United States
(12) Patent Application Publication
(10) Pub. No.: US 2024/0287160 A1
(43) Pub. Date: Aug. 29, 2024

(54) ANTIBODY COCKTAIL FOR TREATMENT OF EBOLAVIRUS INFECTIONS

(71) Applicant: VANDERBILT UNIVERSITY, Nashville, TN (US)

(72) Inventors: James E. CROWE, Jr., Nashville, TN (US); Pavlo GILCHUK, Nashville, TN (US)

(21) Appl. No.: 18/556,648

(22) PCT Filed: Apr. 20, 2022
(86) PCT No.: PCT/US2022/025536

§ 371 Date: Oct. 20, 2023

Related U.S. Application Data
(60) Provisional application No. 63/177,528, filed on Apr. 21, 2021.

(51) Int. Cl. C07K 16/10; A61K 39/00; A61P 31/14

(52) U.S. Cl. CPC C07K 16/10; A61P 31/14; A61K 2039/507; C07K 2317/24; C07K 2317/34; C07K 2317/52; C07K 2317/72; C07K 2317/732; C07K 2317/76

(57) ABSTRACT

The present disclosure is directed to an antibody cocktail comprising antibodies that bind to and protect subjects again multiple ebolavirus strains and methods for use thereof.

### PRIORITY CLAIM

This application provisional application No. 63/177,528 filed on Apr. 21, 2021, the entire contents of which are hereby incorporated by reference.

### FEDERAL FUNDING STATEMENT

This invention was made with government support under Grant nos. U19 AI109711 and U19 AI142785 awarded by National Institutes of Health, Contract HHSN272201400058C awarded by Health and Human Services, and Grant No. HDTRA1-13-1-0034 awarded by the Defense Threat Reduction Agent. The government has certain rights in the invention.

### BACKGROUND

### 1. Field of the Disclosure

The present disclosure relates generally to the fields of medicine, infectious disease, and immunology. More particular, the disclosure relates to human antibodies binding to ebola virus and methods of use therefor.

### 2. Background

The Filoviridae family consists of six antigenically distinct species, including Zaire ebolavirus (represented by Ebola virus [EBOV]), Sudan ebolavirus (Sudan virus [SUDV]), Bundibugyo ebolavirus (Bundibugyo virus [BDBV]), Taï Forest ebolavirus (Taï Forest virus [TAFV]), Reston ebolavirus (Reston virus [RESV]) (Feldmann et al., 2020; Kuhn et al., 2019), and Bombali ebolavirus (Bombali virus [BOMV])(Goldstein et al., 2019). Three ebolaviruses—EBOV, BDBV, and SUDV, are responsible for severe disease and occasional deadly outbreaks in Africa posing a significant health threat. A total of 19 confirmed ebolavirus disease (EVD) outbreaks caused by EBOV have occurred, with >30,000 people infected to date and an average reported mortality rate of ˜70%. In 2021, there are ongoing EVD outbreaks in the Democratic Republic of the Congo (DRC) and Guinea (WHO, 2021). BDBV has caused two confirmed outbreaks and infected 206 people (˜32% mortality rate), and SUDV has been responsible for eight confirmed outbreaks and infected 779 people (˜53% mortality rate) (WHO, 2021). The largest EVD epidemic to date occurred in 2013-2016 in West Africa with a total of 28,610 disease cases and 11,308 deaths reported (WHO, 2021), highlighting the urgent need for development of medical countermeasures. Monoclonal antibody (mAb) therapies have demonstrated safety and significant survival benefit in the treatment of acute EVD caused by EBOV in randomized controlled human trials (Gaudinski et al., 2019; Levine, 2019; Mulangu et al., 2019; Sivapalasingam et al., 2018), and several investigational human mAb treatments have been shown to reverse the advanced EVD in non-buman primates caused by EBOV (Bornholdt et al., 2019; Corti et al., 2016; Gilchuk et al., 2020b; Pascal et al., 2018; Qiu et al., 2014). BDBV (Bornholdt et al., 2019; Gilchuk et al., 2018b), or SUDV (Bornholdt et al., 2019; Herbert et al., 2020). By 2020, two mAb-based therapeutics-ansuvimab-zykl (Ebanga) and atoltivimab+maftivimab+odesivimab-ebgn (Inmazeb)—have been developed and approved by the Food and Drug Administration (FDA) for clinical use (FDA, 2020a, b). Both of these approved antibody treatments are monospecific to EBOV, and therefore, not indicated for treatment of BDBV or SUDV infection. Identification of mAbs that cross-neutralize EBOV, BDBV, and SUDV with high potency is challenging due to the relatively high antigenic variability between these viruses (King et al., 2019). The efficacy of previously reported investigational antibody therapeutics typically is limited to only one of the three medically important ebolavirus species. The nature of future ebolavirus outbreaks cannot be predicted, however, and in a scenario of global spread viruses can mutate rapidly making available antibody treatments vulnerable to escape, as has been recently shown for SARS-COV-2 (Starr et al., 2021; Wang et al., 2021). One approach could be to develop separate therapeutic antibody products for BDBV or SUDV or future escape variants of EBOV. It is desirable from a practical standpoint, however, to identify a single broad therapeutic spectrum antibody treatment of an equivalent or higher potency to existing monospecific antibody treatments that could be used for treatment of EBOV, BDBV, or SUDV. Therefore, ongoing efforts are needed to increase the therapeutic breadth of antibody therapies while maintaining or improving efficacy.

The ebolavirus envelope contains a single surface protein, the glycoprotein (GP), which is the key target for neutralizing mAbs (King et al., 2018; Lee et al., 2008; Lee and Saphire, 2009; Misasi and Sullivan, 2021). The inventors previously described isolation of two broadly neutralizing human antibodies designated EBOV-515 and EBOV-442 using a human B cell hybridoma approach (Gilchuk et al., 2018a). Each of these two mAbs exhibited favorable immunological profiles, which included (1) broad reactivity for binding to GP of diverse species (EBOV, BDBV, and SUDV), (2) broad neutralization of authentic ebolaviruses (EBOV, BDBV, and SUDV), (3) recognition of distinct, non-overlapping epitopes in the GP (EBOV-515 is base-specific, and EBOV-442 is glycan-cap-specific), and (4) a high level of therapeutic protection against EBOV in mice. The antibodies EBOV-515 or EBOV-442 are analogous to the broadly reactive mAb EBOV-520 (GP-base-specific mAb) or mAb EBOV-548 (glycan-cap-specific), respectively. The inventors recently described a beneficial feature of the combination of EBOV-520+EBOV-548 by showing that these two antibodies synergized for virus neutralization when combined in a cocktail and conferred therapeutic protection in EBOV-challenged rhesus macaques (Gilchuk et al., 2020b). They did not previously characterize the similar combination of EBOV-515+EBOV-442. However, the inventors' previous studies revealed that the individual antibodies EBOV-515 and EBOV-442 had higher potency to neutralize the most antigenically distinct of the three viruses (SUDV) when compared, respectively, to the potency of EBOV-520 or EBOV-548. Also, EBOV-515 monotherapy in mice demonstrated a high level of therapeutic protection against SUDV (Gilchuk et al., 2018a; Gilchuk et al., 2020b).

### SUMMARY

Thus, in accordance with the present disclosure, there is provided a method of treating a subject infected with ebola virus, or reducing the likelihood of infection of a subject at risk of contracting ebola virus, comprising delivering to said subject at least first and second structurally distinct antibodies or antibody fragments, wherein said first antibody or antibody fragment has heavy and light chain CDR1-3 sequences of SEQ ID NOS: 5-7 and 8-10, respectively, and the second antibody or antibody fragment has heavy and light chain CDR1-3 sequences of SEQ ID NOS: 15-17 and 18-20, respectively. The first antibody or antibody fragment may be encoded by heavy and light chain sequences of SEQ ID NOS: 1 and 2, respectively, and the second antibody or antibody fragment may be encoded by heavy and light chain sequences of SEQ ID NOS; 11 and 12, respectively; the first antibody or antibody fragment may be encoded by heavy and light chain sequences having 95% identity to SEQ ID NOS: 1 and 2, respectively, and the second antibody or antibody fragment may be encoded by heavy and light chain sequences having 95% identity to SEQ ID NOS: 11 and 12, respectively; or the first antibody or antibody fragment may be encoded by heavy and light chain sequences having 70%, 80%, or 90% sequence identity to SEQ ID NOS: 1 and 2, respectively, and the second antibody or antibody fragment may be encoded by heavy and light chain sequences having 70%, 80%, or 90% sequence identity to SEQ ID NOS: 11 and 12, respectively. The first antibody or antibody fragment may comprise heavy and light chain sequences of SEQ ID NOS: 3 and 4, respectively, and the second antibody or antibody fragment may comprise heavy and light chain sequences of SEQ ID NOS: 13 and 14, respectively; the first antibody or antibody fragment may comprise heavy and light chain sequences having 70%, 80% or 90% sequence identity to SEQ ID NOS: 3 and 4, respectively, and the second antibody or antibody fragment may comprise heavy and light chain sequences having 70%, 80%, or 90% sequence identity to SEQ ID NOS: 13 and 14, respectively; or the first antibody or antibody fragment may comprise heavy and light chain sequences having 95% sequence identity to SEQ ID NOS: 3 and 4, respectively, and the second antibody or antibody fragment may comprise heavy and light chain sequences having 95% sequence identity to SEQ ID NOS: 13 and 14, respectively. At least one of said antibody fragments may be a recombinant scFv (single chain fragment variable) antibody, Fab fragment, F(ab′)₂ fragment, or Fv fragment. At least one of said antibodies may be an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern. At least one of said antibodies may be a chimeric antibody or a bispecific antibody. At least one of said antibodies or antibody fragments may be administered prior to infection or after infection. The subject may have been diagnosed with an ebola virus infection. Delivering may comprise antibody or antibody fragment administration, or genetic delivery with an RNA or DNA sequence or vector encoding the antibody or antibody fragment.

Also provide is a composition comprising at least first and second structurally distinct antibodies or antibody fragments, wherein said first antibody or antibody fragment has heavy and light chain CDR1-3 sequences of SEQ ID NOS: 5-7 and 8-10, respectively, wherein said second antibody or antibody fragment has heavy and light chain CDR1-3 sequences of SEQ ID NOS: 15-17 and 18-20, respectively. The first antibody or antibody fragment may be encoded by heavy and light chain sequences of SEQ ID NOS: 1 and 2, respectively, and the second antibody or antibody fragment may be encoded by heavy and light chain sequences of SEQ ID NOS; 11 and 12, respectively; the first antibody or antibody fragment may be encoded by heavy and light chain sequences having 95% identity to SEQ ID NOS: 1 and 2, respectively, and the second antibody or antibody fragment may be encoded by heavy and light chain sequences having 95% identity to SEQ ID NOS: 11 and 12, respectively; or the first antibody or antibody fragment may be encoded by heavy and light chain sequences having 70%, 80%, or 90% sequence identity to SEQ ID NOS: 1 and 2, respectively, and the second antibody or antibody fragment may be encoded by heavy and light chain sequences having 70%, 80%, or 90% sequence identity to SEQ ID NOS: 11 and 12, respectively. The first antibody or antibody fragment may comprise heavy and light chain sequences of SEQ ID NOS: 3 and 4, respectively, and the second antibody or antibody fragment may comprise heavy and light chain sequences of SEQ ID NOS: 13 and 14, respectively; the first antibody or antibody fragment may comprise heavy and light chain sequences having 70%, 80% or 90% sequence identity to SEQ ID NOS: 3 and 4, respectively, and the second antibody or antibody fragment may comprise heavy and light chain sequences having 70%, 80%, or 90% sequence identity to SEQ ID NOS: 13 and 14, respectively; or the first antibody or antibody fragment may comprise heavy and light chain sequences having 95% sequence identity to SEQ ID NOS: 3 and 4, respectively, and the second antibody or antibody fragment may comprise heavy and light chain sequences having 95% sequence identity to SEQ ID NOS: 13 and 14, respectively. At least one of said antibody fragments may be a recombinant scFv (single chain fragment variable) antibody, Fab fragment, F(ab′)₂ fragment, or Fv fragment. At least one of said antibodies may be an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern. At least one of said antibodies may be a chimeric antibody or a bispecific antibody. At least one of said antibodies or antibody fragments may further comprise a cell penetrating peptide and/or is an intrabody.

In another embodiment, there is provided an engineered cell encoding at least first and second structurally distinct antibodies or antibody fragments, wherein said first antibody or antibody fragment has heavy and light chain CDR1-3 sequences of SEQ ID NOS: 5-7 and 8-10, respectively, wherein said second antibody or antibody fragment has heavy and light chain CDR1-3 sequences of SEQ ID NOS: 15-17 and 18-20, respectively. The first antibody or antibody fragment may be encoded by heavy and light chain sequences of SEQ ID NOS: 1 and 2, respectively, and the second antibody or antibody fragment may be encoded by heavy and light chain sequences of SEQ ID NOS; 11 and 12, respectively; the first antibody or antibody fragment may be encoded by heavy and light chain sequences having 95% identity to SEQ ID NOS: 1 and 2, respectively, and the second antibody or antibody fragment may be encoded by heavy and light chain sequences having 95% identity to SEQ ID NOS: 11 and 12, respectively; or the first antibody or antibody fragment may be encoded by heavy and light chain sequences having 70%, 80%, or 90% sequence identity to SEQ ID NOS: 1 and 2, respectively, and the second antibody or antibody fragment may be encoded by heavy and light chain sequences having 70%, 80%, or 90% sequence identity to SEQ ID NOS: 11 and 12, respectively. The first antibody or antibody fragment may comprise heavy and light chain sequences of SEQ ID NOS: 3 and 4, respectively, and the second antibody or antibody fragment may comprise heavy and light chain sequences of SEQ ID NOS: 13 and 14, respectively; the first antibody or antibody fragment may comprise heavy and light chain sequences having 70%, 80% or 90% sequence identity to SEQ ID NOS: 3 and 4, respectively, and the second antibody or antibody fragment may comprise heavy and light chain sequences having 70%, 80%, or 90% sequence identity to SEQ ID NOS: 13 and 14, respectively; or the first antibody or antibody fragment may comprise heavy and light chain sequences having 95% sequence identity to SEQ ID NOS: 3 and 4, respectively, and the second antibody or antibody fragment may comprise heavy and light chain sequences having 95% sequence identity to SEQ ID NOS: 13 and 14, respectively. At least one of said antibody fragments may be a recombinant scFv (single chain fragment variable) antibody, Fab fragment, F(ab′)₂ fragment, or Fv fragment. At least one of said antibodies may be an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern. At least one of said antibodies may be a chimeric antibody or a bispecific antibody. At least one of said antibodies or antibody fragments may further comprise a cell penetrating peptide and/or is an intrabody.

In yet another embodiment, there is provided a vaccine formulation comprising at least first and second structurally distinct antibodies or antibody fragments, wherein said first antibody or antibody fragment has heavy and light chain CDR1-3 sequences of SEQ ID NOS: 5-7 and 8-10, respectively, wherein said second antibody or antibody fragment has heavy and light chain CDR1-3 sequences of SEQ ID NOS: 15-17 and 18-20, respectively. The first antibody or antibody fragment may be encoded by heavy and light chain sequences of SEQ ID NOS: 1 and 2, respectively, and the second antibody or antibody fragment may be encoded by heavy and light chain sequences of SEQ ID NOS; 11 and 12, respectively; the first antibody or antibody fragment may be encoded by heavy and light chain sequences having 95% identity to SEQ ID NOS: 1 and 2, respectively, and the second antibody or antibody fragment may be encoded by heavy and light chain sequences having 95% identity to SEQ ID NOS: 11 and 12, respectively; or the first antibody or antibody fragment may be encoded by heavy and light chain sequences having 70%, 80%, or 90% sequence identity to SEQ ID NOS: 1 and 2, respectively, and the second antibody or antibody fragment may be encoded by heavy and light chain sequences having 70%, 80%, or 90% sequence identity to SEQ ID NOS: 11 and 12, respectively. The first antibody or antibody fragment may comprise heavy and light chain sequences of SEQ ID NOS: 3 and 4, respectively, and the second antibody or antibody fragment may comprise heavy and light chain sequences of SEQ ID NOS: 13 and 14, respectively; the first antibody or antibody fragment may comprise heavy and light chain sequences having 70%, 80% or 90% sequence identity to SEQ ID NOS: 3 and 4, respectively, and the second antibody or antibody fragment may comprise heavy and light chain sequences having 70%, 80%, or 90% sequence identity to SEQ ID NOS: 13 and 14, respectively; or the first antibody or antibody fragment may comprise heavy and light chain sequences having 95% sequence identity to SEQ ID NOS: 3 and 4, respectively, and the second antibody or antibody fragment may comprise heavy and light chain sequences having 95% sequence identity to SEQ ID NOS: 13 and 14, respectively. At least one of said antibody fragments may be a recombinant scFv (single chain fragment variable) antibody, Fab fragment, F(ab′)₂ fragment, or Fv fragment. At least one of said antibodies may be an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern. At least one of said antibodies may be a chimeric antibody or a bispecific antibody. At least one of said antibodies or antibody fragments further comprises a cell penetrating peptide and/or is an intrabody.

In yet another embodiment, there is provided a vaccine formulation comprising one or more expression vectors encoding at least first and second structurally distinct antibodies or antibody fragments, wherein said first antibody or antibody fragment has heavy and light chain CDR1-3 sequences of SEQ ID NOS: 5-7 and 8-10, respectively, wherein said second antibody or antibody fragment has heavy and light chain CDR1-3 sequences of SEQ ID NOS: 15-17 and 18-20, respectively. The first antibody or antibody fragment may be encoded by heavy and light chain sequences of SEQ ID NOS: 1 and 2, respectively, and the second antibody or antibody fragment may be encoded by heavy and light chain sequences of SEQ ID NOS; 11 and 12, respectively; the first antibody or antibody fragment may be encoded by heavy and light chain sequences having 95% identity to SEQ ID NOS: 1 and 2, respectively, and the second antibody or antibody fragment may be encoded by heavy and light chain sequences having 95% identity to SEQ ID NOS: 11 and 12, respectively; or the first antibody or antibody fragment may be encoded by heavy and light chain sequences having 70%, 80%, or 90% sequence identity to SEQ ID NOS: 1 and 2, respectively, and the second antibody or antibody fragment may be encoded by heavy and light chain sequences having 70%, 80%, or 90% sequence identity to SEQ ID NOS: 11 and 12, respectively. The first antibody or antibody fragment may comprise heavy and light chain sequences of SEQ ID NOS: 3 and 4, respectively, and the second antibody or antibody fragment may comprise heavy and light chain sequences of SEQ ID NOS: 13 and 14, respectively; the first antibody or antibody fragment may comprise heavy and light chain sequences having 70%, 80% or 90% sequence identity to SEQ ID NOS: 3 and 4, respectively, and the second antibody or antibody fragment may comprise heavy and light chain sequences having 70%, 80%, or 90% sequence identity to SEQ ID NOS: 13 and 14, respectively; or the first antibody or antibody fragment may comprise heavy and light chain sequences having 95% sequence identity to SEQ ID NOS: 3 and 4, respectively, and the second antibody or antibody fragment may comprise heavy and light chain sequences having 95% sequence identity to SEQ ID NOS: 13 and 14, respectively.

At least one of said antibody fragments may be a recombinant scFv (single chain fragment variable) antibody, Fab fragment, F(ab′)₂ fragment, or Fv fragment. At least one of said antibodies may be an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern. At least one of said antibodies may be a chimeric antibody or a bispecific antibody. At least one of said antibodies or antibody fragments may further comprise a cell penetrating peptide and/or is an intrabody. The expression vector(s) may be Sindbis virus or VEE vector(s). The vaccine may be formulated for delivery by needle injection, jet injection, or electroporation. The vaccine may further comprise one or more expression vectors encoding for a third antibody or antibody fragment.

In a further embodiment, there is provided a method of protecting the health of a placenta and/or fetus of a pregnant a subject infected with or at risk of infection with ebola virus comprising delivering to said subject a dual antibody composition as described herein. The composition may reduce viral load and/or pathology of the fetus as compared to an untreated control.

In yet a further embodiment, there is provided a composition comprising at least two human monoclonal antibodies or antibody fragments, wherein a first antibody binds to ebola virus glycan-cap and a second antibody binds to the ebola virus GP-base, wherein said antibodies together protect against EBOV, BDBV, and SUDV. The Fc region of at least one of the first antibody and second antibody may contain a LALA-PG mutation.

Also provided is a bispecific antibody comprising heavy and light chain CDR1-3 sequences of SEQ ID NOS: 5-7 and 8-10, respectively and heavy and light chain CDR1-3 sequences of SEQ ID NOS: 15-17 and 18-20, respectively. Methods of treating a subject infected with ebola virus, or reducing the likelihood of infection of a subject at risk of contracting ebola virus, comprising delivering to said subject such bispecific antibody are also contemplated. Additionally, an engineered cell that expresses such a bispecific antibody is disclosed.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-D. Functional activities of pan-ebolavirus cocktail candidate antibodies rEBOV-442 and rEBOV-515. (FIG. 1A) EBOV, BDBV, or SUDV neutralization. Biosafety level 4 recombinant ebolaviruses encoding enhanced green fluorescent protein (eGFP) were incubated with increasing concentrations of recombinantly produced purified mAbs, and infection was determined at 3 days after inoculation by measuring eGFP fluorescence in cells. Mean±SD of technical triplicates from one experiment are shown. (FIG. 1B) In vitro killing capacity mediated by the Fc regions of IgG1-engineered variants of mAbs measured by rapid fluorometric antibody-mediated cytotoxicity (RFADCC) assay. Human PBMCs (effector cells) were incubated with a SNAP-tagged EBOV GP-expressing 293F cell line as a target in the presence of increasing concentrations of purified recombinant mAbs, and cytotoxic activity was measured by flow cytometry. Dengue virus human antibody rDENV 2D22 served as a negative control. The dotted line indicates assay background. Mean±SD of technical duplicates from one experiment are shown. (FIG. 1C) Heat map summarizing reactivity breadth and potency of rEBOV-442 and rEBOV-515. * indicates data determined in the inventors' previous work using hybridoma-cell-secreted mAbs (Gilchuk et al., 2018). (FIG. 1D) Results of viral selections with individual antibodies or the cocktail, indicating the number of replicates with escape out of total number tested, resistance of selected escape variants to individual antibodies or the cocktail, and the selected mutations in the GP that escape neutralization by rEBOV-442. The escape selection was performed using infectious chimeric VSV expressing EBOV (Mayinga variant) GP. ** indicates the prevalent mutation L273P that was identified in all sequenced escape viruses. See also FIGS. S1A-C.

FIGS. 2A-C. Differential requirements for the Fc regions of antibodies rEBOV-442 and rEBOV-515 for optimal therapeutic protection in mice. C57BL/6 mice were challenged with mouse-adapted EBOV-MA, treated at 1 dpi with wild-type IgG1 or the Fc-effector function disabled IgG1 LALA-PG variant of antibodies rEBOV-442 (5 mg/kg) and rEBOV-515 (1 mg/kg), and monitored for 28 days. (FIG. 2A) Kaplan-Meier survival plot. The overall difference in survival between the groups was estimated using two-sided log-rank (Mantel-Cox) test. (FIG. 2B) Weight change. (FIG. 2C) Clinical score. +, animal found dead prior to reaching the pre-determined clinical score. Mean values are shown in FIGS. 2B-C, and data represent one experiment with five mice per group. Dengue virus-specific antibody rDENV 2D22 was used as a control. A historical control for protection from weight loss with 5 mg/kg of wild-type rEBOV-515 treatment (dotted line in FIG. 2B) is shown for comparative purposes.

FIGS. 3A-C. Broad and synergistic neutralizing activity mediated by the cocktail of rEBOV-442 and rEBOV-515. Neutralizing activity of individual antibodies or their mixture was assessed using infectious chimeric VSV expressing EBOV (Mayinga variant), BDBV (Uganda variant), or SUDV (Boniface variant) GP and real-time cell analysis (RTCA) assay. (FIG. 3A) Neutralization of VSV/EBOV GP, VSV/BDBV GP, or VSV/SUDV GP by rEBOV-442 alone, rEBOV-515 alone, or a 1:1 mixture of rEBOV-442 and rEBOV-515. (FIG. 3B) Serially diluted rEBOV-442 was titrated into serially-diluted rEBOV-515 to generate a pairwise combinatorial matrix of two antibodies in the mixture. The matrix shows neutralization dose-response data for VSV/EBOV GP, VSV/BDBV GP, or VSV/SUDV GP, by indicated concentrations of rEBOV-442 and rEBOV-515. Axes denote the concentration of each antibody, with the percent neutralization shown in each square. The heat map denotes a gradient of 0 (white) to 100% (red) neutralization. Examples of neutralization by rEBOV-515 alone (raspberry box) or rEBOV-442 alone (blue box) in comparison to a combined indicated concentration of two mAbs in the cocktail (green box) are shown. (FIG. 3C) Synergy distribution map generated from the dose-response neutralization matrix in FIG. 3B. Red color indicates areas in which synergistic neutralization was observed; shaded grey box indicates the area of maximum synergy between the two monoclonal antibodies, and the δ-score for this area is shown. The d-score is a synergy score: values <−10 indicate antagonism; values-10 to 10 indicate an additive effect; values >10 indicate synergy. Data in FIGS. 3A-C) are from a representative experiment performed in technical duplicate and repeated twice. See also FIGS. S2A-C.

FIGS. 4A-C. The cocktail treatment provides pan-ebolavirus protection of nonhuman primates against disease. Rhesus macaques were inoculated with a lethal dose of the EBOV Kikwit or SUDV/Gulu viruses intramuscularly (i.m.) on day 0 and were treated with total 30 mg/kg of the cocktail (1:2 mixture of rEBOV-442 and rEBOV-515) intravenously on 3 and 6 dpi (EBOV/Kikwit; n=5 per cohort), or 4 and 7 dpi (SUDV/Gulu; n=5 per cohort). Cynomolgus monkeys were inoculated with a lethal dose of the BDBV/Uganda i.m, on day 0 and were treated with a total dose of 30 mg/kg of the cocktail (1:2 mixture of rEBOV-442 and rEBOV-515) intravenously on 6 and 9 dpi (n=5 per cohort). The contemporaneous control was an untreated NHP challenged with the virus (n=1 for each cohort). One experiment was performed. (FIG. 4A) Kaplan-Meier survival plot. The historical untreated controls (grey) are shown for comparative purposes (see Methods). The proportion surviving at day 28 after viral challenge in the treated cohort was compared to the respective historical untreated cohort using a 2-sided exact unconditional test of homogeneity. (FIG. 4B) Clinical score. (FIG. 4C) Selected blood chemistry measurements: ALP, alkaline phosphatase; GGT, gamma-glutamyl transpeptidase; CRE, creatinine. Antibody treatment times are indicated with blue dotted vertical lines. Orange curves indicate treated, and black indicate untreated animals in FIGS. 4A-C. The black dotted line in FIG. 4B indicates the clinical score threshold for euthanasia. See also Tables S1-6.

FIGS. 5A-B. The cocktail treatment provides pan-ebolavirus protection of nonhuman primates against viremia. Blood viral loads were assessed from individual animals that were challenged with EBOV/Kikwit, BDBV/Uganda, or SUDV/Gulu and treated with the two-antibody cocktail as described in FIGS. 4A-C. (FIG. 5A) Kinetics of blood viral load determined for genome equivalents (GEq) using qRT-PCR. (FIG. 5B) Kinetics of infectious virus blood viral load as determined by plaque assay. Orange curves indicate treated, and black indicate untreated animals. Antibody treatment times are indicated with blue dotted vertical lines. The black dotted line indicates the limit of detection (LOD), which was 3.7 log₁₀ GEq/mL (FIG. 5A) or 5 PFU/mL (FIG. 5B). Each measurement in FIGS. 5A-B represents the mean of technical duplicates. See also FIG. S3.

FIGS. 6A-F. rEBOV-515 binds to the major site of vulnerability in the GP base region in a distinct manner. (FIG. 6A) Cryo-EM structure of rEBOV-442 (heavy chain in blue and light chain in grey) and rEBOV-515 (heavy chain in maroon and light chain in pink) Fab bound to EBOV GPΔMuc/Mak. A side view in relation to the viral membrane is shown. Fab constant domains were excluded by masking. (FIG. 6B) The predicted contact surface of broadly neutralizing antibodies rEBOV-515 and rEBOV-520 on the surface representation of the EBOV GPΔMuc/Mak monomer model (PDB: 5JQ3). Non-overlapping contact surfaces for rEBOV-515 or rEBOV-520 are shown, respectively, in maroon or orange, and overlapping contact surface of both antibodies is shown in yellow. (FIG. 6C) Contact residue details of the rEBOV-515 heavy chain Fab interactions with the base of GP. CDRH3 contacts include a backbone-mediated hydrogen bond at S102_{H3} to N512_{GP2}, a key contact that links the β17-β18 loop to the base of the IFL via W291 in unliganded GP1. Contacts near the 310 pocket include a potential hydrogen bond between S105_{H3} and E106_{GP1} that forms when a large portion of the CDRH3 loop displaces the β17-β18 loop. Within the CDRH2 loop, Y52_{H2} and Y55_{H2} make potential hydrogen bonds via their hydroxyl groups to H549_{GP2} or H516_{GP2} on GP2, respectively. One of the most extensive rEBOV-515 contacts is from W103_{H3}, which forms a strong cation-pi bond with R136_{GP1}, allowing R136 to make additional hydrogen bonds with Y3_{H1} and a salt bridge with E106H3. (FIG. 6D) Epitope details of the rEBOV-515 light chain interactions with the base of GP. The rEBOV-515 light chain makes contacts with all three CDRs exclusively within GP2. Contact features include a potential hydrogen bond between N32_{L1} and the backbone of C511_{GP2}, and a salt bridge between D5012 and K510_{GP2}. Pi cation interactions at W9413 with H549GP2 (black dashed line) and an additional potential hydrogen bond at N92_{L3} with N550_{GP2} provide additional stabilizing interactions. (FIG. 6E) Comparison of antibody CDRH3 loops that bind in and around the 310 pocket (blue dashed circle) and putative glycerol pocket (solid yellow line). rEBOV-520 and ADI-15946 replace and mimic residue W291_{GP1} (that anchors down the β17-β18 loop in apo-GP) with a tryptophan from their CDRH3 loops. rEBOV-515 uses an analogous tryptophan residue (W103_{H3}) to contact R136_{GP1} via a strong cation-pi interactions bond, which causes a shift in the rotamer of R136_{GP1}. W103_{H3} from rEBOV-515 also accesses a pocket that is occupied by a glycerol cryoprotectant molecule in the unliganded crystal structure of GP, which is also occupied by Y28_{L1} from ADI-15946. (FIG. 6F) A shift in the placement of the GP1 α2 helix that cased by the CDRH3 from rEBOV-520 but is lacking in rEBOV-515 binding due to a shorter CDR loop is shown. Red dotted lines: hydrogen bonds; green dashed lines: salt bridge; purple dashed line: cation-pi interaction; black dashed line: carbon-pi or aromatic interactions. rEBOV-520-GP PDB: 6UYE; ADI-15946-GP PDB: 6MAM. See also FIGS. S4A-B, S5A-C, -6 and Table S7.

FIG. 7. Conservation of the binding sites for broadly reactive antibodies rEBOV-515, rEBOV-520, and ADI-15946. Apo-GP is surface-rendered according to residue conservation on the left, with no conservation in dark purple (0%) and complete conservation in white (100%). The corresponding antibody footprints are highlighted. On the right are aligned sequences of the interacting regions on GP from the three major ebolaviruses, EBOV, BDBV, and SUDV. Total contacts for each residue at 4 Å distance or less were determined (see Table S7) and residues are highlighted in blue according to the number of contacts, with darker blue indicating more contacts and thus a higher likelihood for contributing critically to binding. Residues that are variable are marked with a green diamond. HC contacts are indicated below in dark purple and LC contacts in pink. (SEQ ID NOS: 24-31)

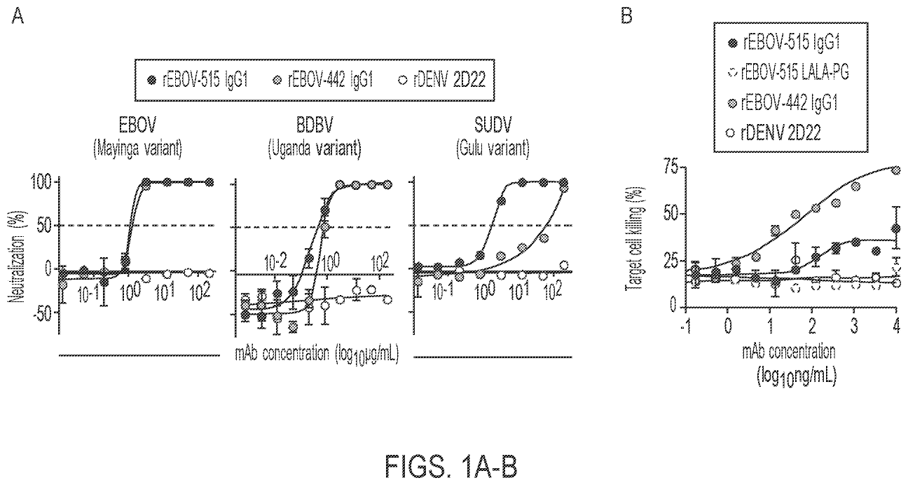

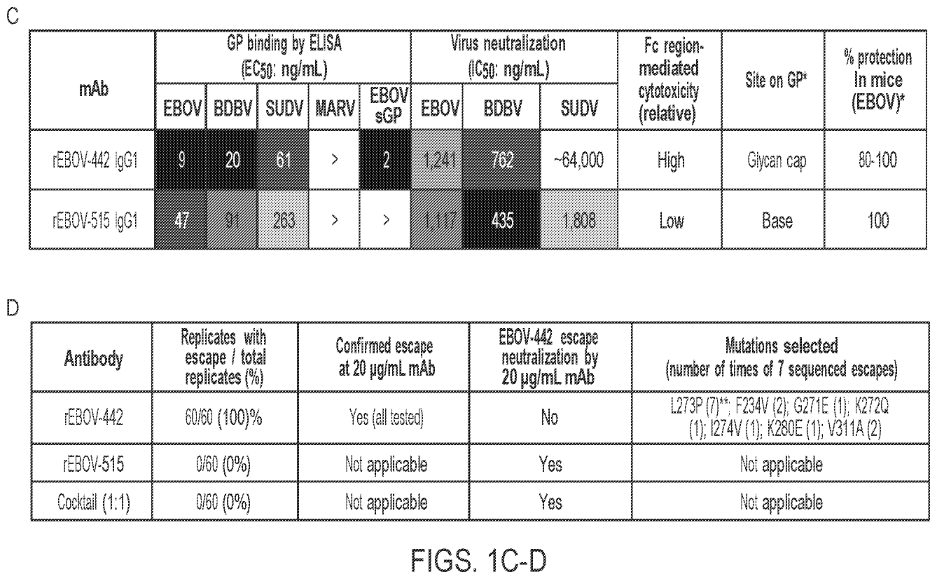

FIGS. S1A-C. Neutralizing activity of antibody rEBOV-515 LALA-PG and neutralization of antibody rEBOV-442-selected escape viruses by rEBOV-515 or the cocktail. (FIG. S1A) EBOV, BDBV, or SUDV neutralization by wild-type IgG1 or IgG1 LALA-PG variants of rEBOV-515. Neutralization was assessed with chimeric Ebola viruses encoding EBOV, DBDV, or SUDV GP as in FIG. 1A. Mean±SD of technical triplicates from one experiment are shown. (FIG. S1B) Real-time cell analysis (RTCA) sensograms for antibody rEBOV-442-selected escape viruses for neutralization by rEBOV-442 alone, rEBOV-515 alone, or a 1:1 antibody cocktail. The escape selection was performed using infectious chimeric VSV expressing EBOV (Mayinga variant) GP. Seven viruses that escaped neutralization by rEBOV-442 and had sequence-verified mutations in the gene encoding the glycan cap region of the GP were incubated in the presence of a saturating concentration (20 μg/mL) of antibody rEBOV-442 (left panel, grey), rEBOV-515 (middle, rose), or a 1:1 mixture of both antibodies. Cytopathic effect (CPE) was monitored kinetically in Vero cells after applying the virus-antibody mixtures to the cells. Sensograms for CPE by the wild-type VSV/EBOV GP without antibody (left panel, red), and full neutralization of the wild-type VSV/EBOV GP by rEBOV-442 (left panel, blue) are shown as the controls. Seven viruses with individual mutations selected by rEBOV-442 and indicated in FIG. 1D, escape neutralization by rEBOV-442 (left panel, grey) but were neutralized by rEBOV-515 (middle panel) or a 1:1 mixture of rEBOV-442 and rEBOV-515 (right panel). (FIG. S1C) Mutations in indicated residues (magenta) identified in the glycan cap region of the GP (PDB: 5JQ3) for rEBOV-442-selected viruses that escaped neutralization by rEBOV-442 as in FIG. S1B. Position of V311 residue is shown with grey sphere. Related to FIGS. 1A-D.

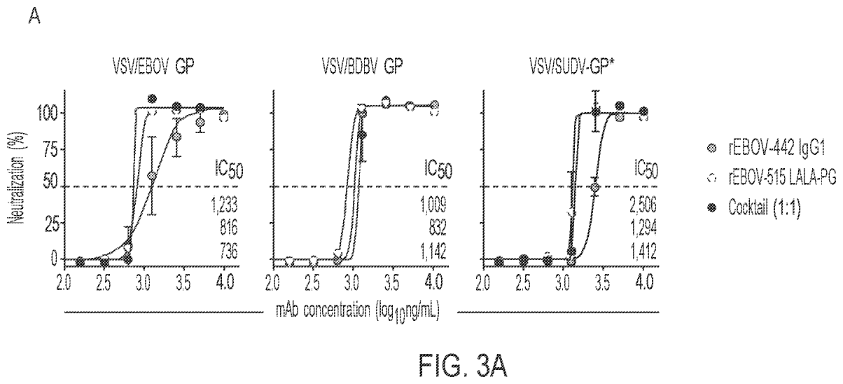

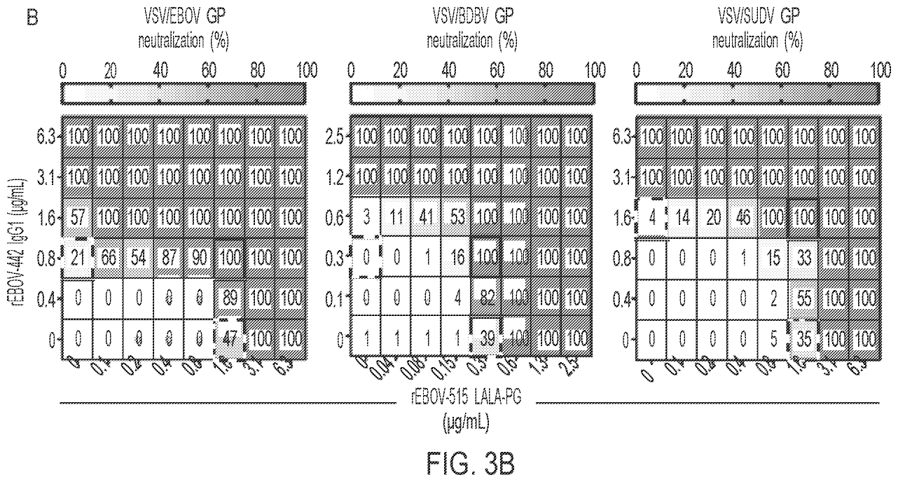

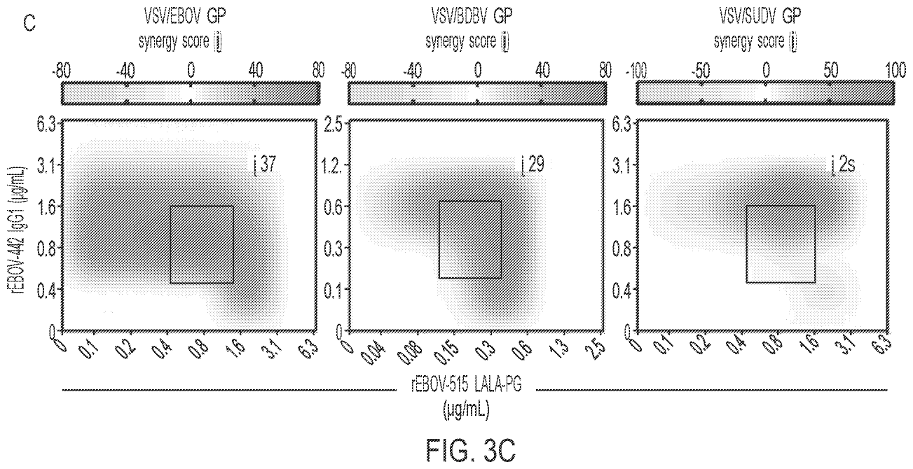

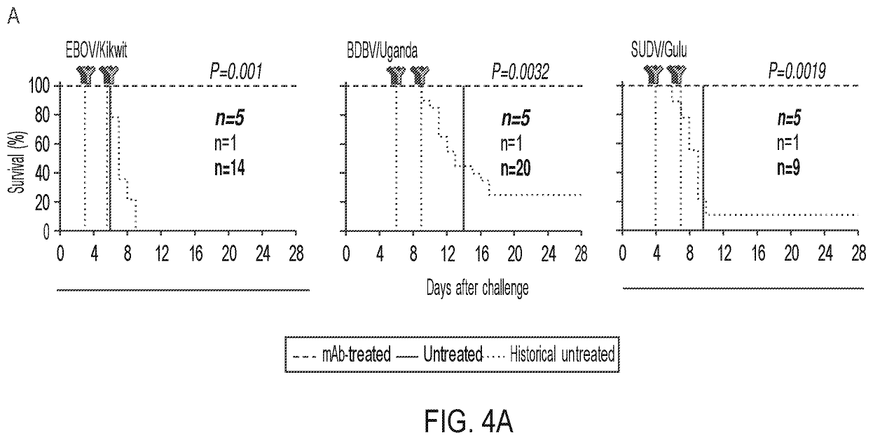

FIGS. S2A-C. Reciprocal synergistic binding activity in the cocktail of rEBOV-442 and rEBOV-515. Serially diluted Alexa Fluor 647-labeled antibody rEBOV-442 IgG1 or rEBOV-515 LALA-PG was titrated into serially-diluted unlabeled partner antibody to generate a pairwise combinatorial matrix of two antibodies in the mixture. Binding to Jurkat EBOV-GP cells was assessed by flow cytometric analysis. (FIG. S2A) Dose-response binding of fluorescently labeled rEBOV-442 alone (left) or rEBOV-515 alone (right) to Jurkat-EBOV GP. Mean±SD of technical duplicates is shown. (FIG. S2B) Binding dose-response matrix by rEBOV-442 or rEBOV-515 bound to the EBOV-GP in the presence of a partner antibody. Axes denote the concentration of each antibody, with the percent binding shown in each square. Binding was calculated as the percent of the maximal median fluorescence intensity signal (MFI) by the highest concentration of respective fluorescently-labeled antibody alone (25 μg/mL) as in FIG. S2A. Examples of an enhanced binding activity by a combination of fluorescently-labeled antibody plus partner antibody at the highest tested concentration (green box) in a comparison to the binding by fluorescently-labeled rEBOV-442 alone (violet box) or rEBOV-515 alone (raspberry box) are shown. (FIG. S2C) Synergy distribution map generated from the dose-response binding matrix in (B). Red color indicates areas in which synergistic neutralization was observed; shaded grey box indicates the area of maximum synergy between the two monoclonal antibodies, and 8-score for this area is shown. 8-score is a synergy score: a value of −10 indicates antagonism; a value of −10 to 10 indicates an additive effect; a value of >10 indicates synergy. Data in FIGS. S2A-B are from a representative experiment performed in technical duplicate and repeated twice. Related to FIGS. 3A-C.

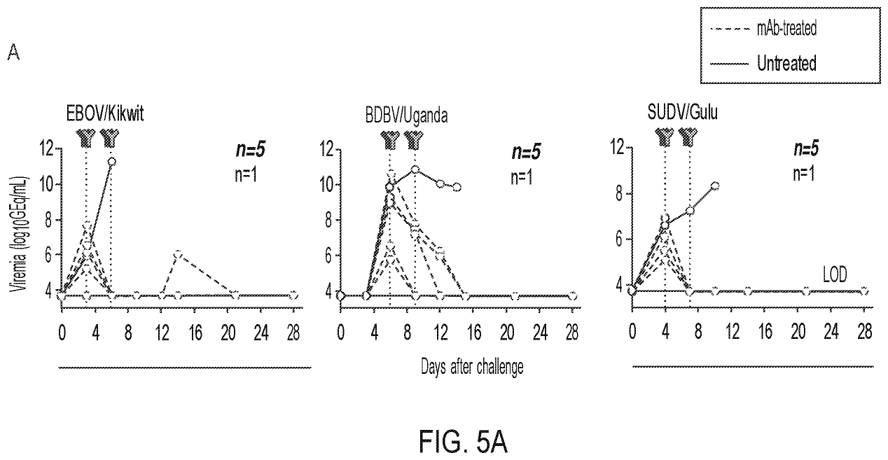

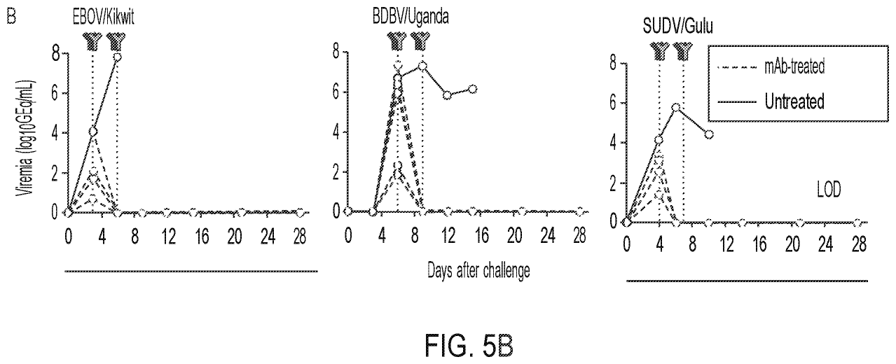

FIG. S3. The cocktail treatment provides pan-ebolavirus protection of nonhuman primates against viremia. Viral RNA load in various peripheral tissues of treated NHPs determined by qRT-PCR (28 dpi for EBOV and SUDV cohort, or 35 dpi for BDBV cohort). Tissues from succumbed untreated NHP from each cohort used as a control. The black dotted line indicates the limit of detection, which was 2.4 log₁₀ GEq/mL. The data are shown as an aligned dot plot with bar, where orange dots indicate measurement from individual treated animals, black dots and bars indicate measurement from untreated control animals, and orange bar indicates the mean value of the measurements for treated cohorts. For samples in which viral RNA was not detected, the measurement values were set to the limit of detection 2.4 log₁₀ GEq/mL. Each measurement represents the mean of technical duplicates. Related to FIGS. 5A-B.

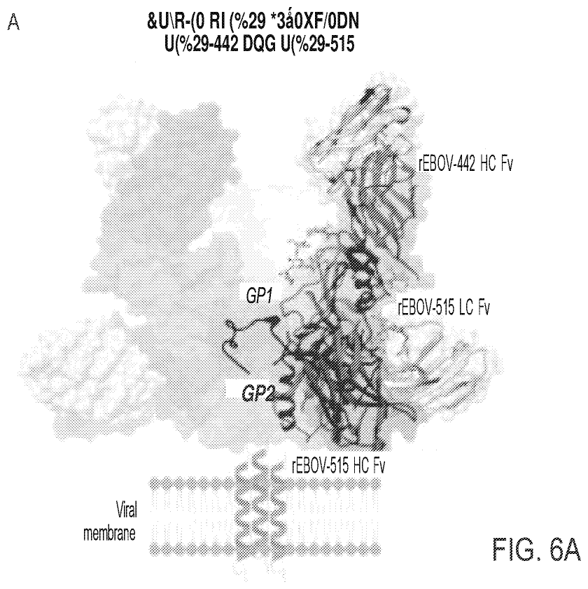

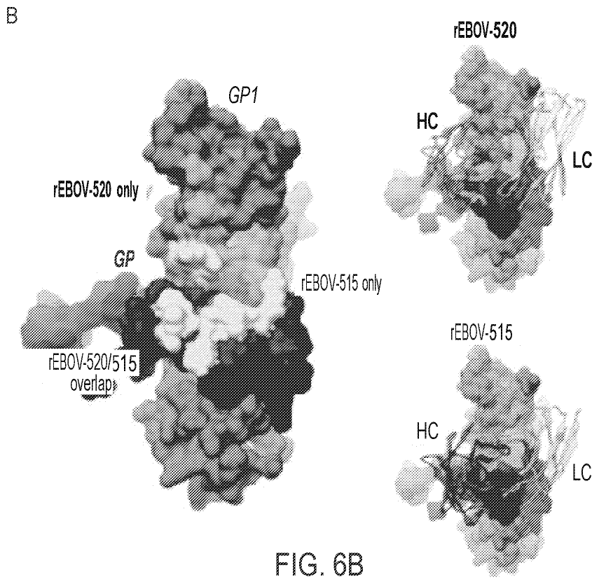

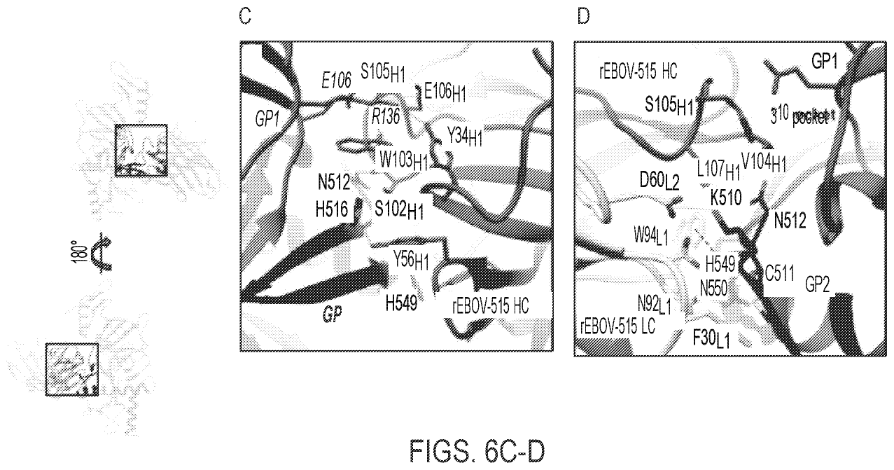

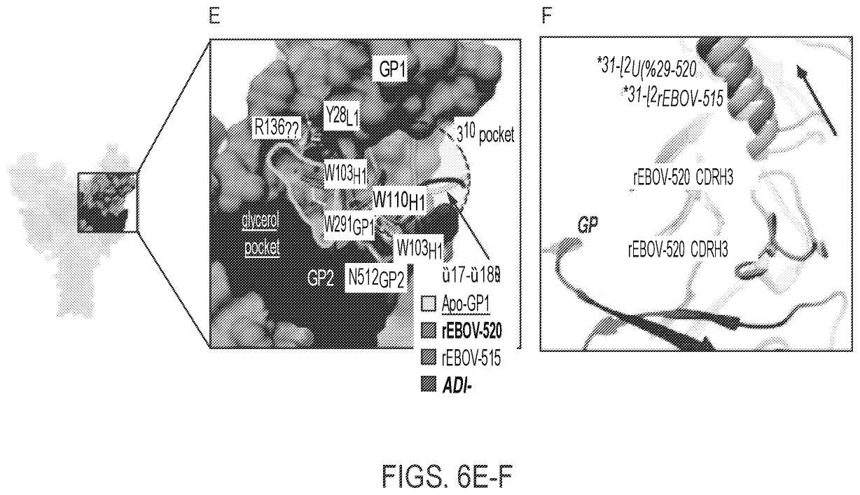

FIGS. S4A-B. Cryo-EM local resolution estimation and FSC curves. (FIG. S4A) The local resolution of the EBOV GPΔMuc/Mak:rEBOV-442/rEBOV-515 cryo-EM map, with local resolutions indicated according to the colors in the legend below. (FIG. S4B) The Fourier shell correlation (FSC) curve for the EBOV GPΔMuc/Mak:rEBOV-442/rEBOV-515 cryo-EM map, generated by Relion 3.1. Related to FIGS. 6A-F.

FIGS. S5A-C. Epitope and paratope binding properties of rEBOV-515. (FIG. S5A) LigPlot schematic showing interaction of rEBOV-515 with GP1 (above) or GP2 (below). Residues that make hydrogen bonds are rendered, and those that contribute van der Waals interactions are drawn as semi-circles. rEBOV-515 CDR loop residues are colored according to the labels, with GP residues in magenta/pink and backbone residues colored in dark cyan. Chain identifiers are in parenthesis. (FIG. S5B) Kyte-Doolittle surface rendering of GP1,2 (left) and a face-on view of the rEBOV-515 Fv paratope (right) with the interacting regions highlighted. Key hydrophobic residues are indicated with important regions of interaction labeled 1-3 on the GP epitope and the corresponding interacting residues labeled 1-3 on the rEBOV-515 paratope. (FIG. S5C) Similar epitope/paratope interaction rendering as in (C) but with Coulombic surface rendering, indicating key electronegative regions on GP that interact with electropositive regions on the rEBOV-515 paratope. Related to FIGS. 6A-F.

FIG. S6. Contact residues for broadly reactive antibodies rEBOV-515, rEBOV-520, and ADI-15946. Base binding antibody contacts on EBOV GP determined by UCSF Chimera with a default cutoff value of −0.4 Å and an allowance of 0 Å (Pettersen et al., 2004). rEBOV-520 from PDB 6PCI (Gilchuk et al., 2020b), ADI-15946 from PDB 6MAM (West et al., 2019). Gradient is green to red, with green being fewer contacts and red being the most contacts. Related to FIGS. 6A-F.

FIGS. S7A-B. rEBOV-515 retains GP-binding capacity at low pH and efficiently inhibits GP cleavage. (FIG. S7A) ELISA binding of rEBOV-515 or rEBOV-520 to the recombinant EBOV, BDBV, or SUDV GP ΔTM at neutral or acidic pH. The mean±SD of technical triplicates and one of two independent experiments are shown. (FIGS. S7B) Cleavage inhibition by the GP-base specific antibodies. Jurkat EBOV-GP cells were pre-incubated with various concentrations of indicated antibodies, treated with thermolysin, then incubated with fluorescently labeled antibody MR78 that recognizes the receptor binding site (RBS) of the GP and assessed for exposure of the RBS on cleaved GP by flow cytometry. Antibody BDBV223 that target a distal antigenic site in the canonical heptad repeat 2 (HR2) region near the membrane proximal external region (MPER) of GP, and dengue virus-specific antibody rDENV 2D22 were used as controls. Results are expressed as the percent of RBS exposure inhibition in the presence of tested mAb relative to controls for minimal binding of labeled MR78 mAb-only to intact (uncleaved) Jurkat EBOV-GP, and maximal binding of labeled MR78 mAb-only to cleaved Jurkat-EBOV GP. Mean±SD of technical triplicates and one of two independent experiments are shown. Related to FIGS. 6A-F.

### DESCRIPTION OF ILLUSTRATIVE EMBODIMENTS

As discussed above, there remains an urgent need to develop improved antibody therapies for the treatment of ebolaviruses with efficacy across various strains. Given the higher potency of rEBOV-515 and rEBOV-442 against SUDV, the inventors tested their combination as a candidate for a “next-generation” broad therapeutic antibody cocktail. In this study, the inventors describe pre-clinical development of the EBOV-515+EBOV-442 antibody cocktail and defined the molecular basis for its pan-ebolavirus activity and efficacy.

These and other aspects of the disclosure are described in detail below.

### I. EBOLAVIRUSES

The genus Ebolavirus is a virological taxon included in the family Filoviridae, order Mononegavirales. The members of this genus are called ebolaviruses. As discussed above, the five known virus species are named for the region where each was originally identified: Bundibugyo ebolavirus, Reston ebolavirus, Sudan ebolavirus, Taï Forest ebolavirus (originally Côte d'Ivoire ebolavirus), and Zaire ebolavirus.

The EBOV protein VP24 inhibits type I and II interferon (IFN) signaling by binding to NPI-1 subfamily karyopherin a (KPNA) nuclear import proteins, preventing their interaction with tyrosine-phosphorylated STAT1 (phospho-STAT1). This inhibits phospho-STAT1 nuclear import. A biochemical screen now identifies heterogeneous nuclear ribonuclear protein complex C1/C2 (hnRNP C1/C2) nuclear import as an additional target of VP24. Co-immunoprecipitation studies demonstrate that hnRNP C1/C2 interacts with multiple KPNA family members, including KPNA1. Interaction with hnRNP C1/C2 occurs through the same KPNAI C-terminal region (amino acids 424-457) that binds VP24 and phospho-STAT1. The ability of hnRNP C1/C2 to bind KPNAI is diminished in the presence of VP24, and cells transiently expressing VP24 redistribute hnRNP C1/C2 from the nucleus to the cytoplasm. These data further define the mechanism of hnRNP C1/C2 nuclear import and demonstrate that the impact of EBOV VP24 on nuclear import extends beyond STAT1.

Ebolaviruses were first described after outbreaks of EVD in southern Sudan in June 1976 and in Zaire in August 1976. The name Ebolavirus is derived from the Ebola River in Zaire (now the Democratic Republic of the Congo), the location of the 1976 outbreak, and the taxonomic suffix-virus (denoting a viral genus). This genus was introduced in 1998 as the “Ebola-like viruses.” In 2002 the name was changed to Ebolavirus and in 2010, the genus was emended. Ebolaviruses are closely related to Marburg viruses, which are included in family Filoviridae as a separate genus.

Researchers have now found evidence of EBOV infection in three species of fruit bats. The bats show no symptoms of the disease, indicating that they might be spreading it. Researchers found that bats of three species-Hypsignathus monstrosus, Epomops franqueti, and Myonycteris torquata-had either genetic material from the EBOV, known as RNA sequences, or evidence of an immune response to the disease. The bats showed no symptoms themselves. Other hosts are possible as well.

### A. Taxonomy

A virus of the family Filoviridae is a member of the genus Ebolavirus if its genome has several gene overlaps, its fourth gene (GP) encodes four proteins (sGP, ssGP, Δ-peptide, and GP1.2) using co-transcriptional editing to express GP1.2 and ssGP and proteolytic cleavage to express sGP and Δ-peptide, peak infectivity of its virions is associated with particles ≈805 nm in length, its genome differs from that of Marburg virus by ≥50% and from that of ebolaviruses by <50% at the nucleotide level, its virions show almost no antigenic cross reactivity with Marburg virions.

The genera Ebolavirus and Marburgvirus were originally classified as the species of the now-obsolete Filovirus genus. In March 1998, the Vertebrate Virus Subcommittee proposed in the International Committee on Taxonomy of Viruses (ICTV) to change the Filovirus genus to the Filoviridae family with two specific genera: Ebola-like viruses and Marburg-like viruses. This proposal was implemented in Washington, D.C., as of April 2001 and in Paris as of July 2002. In 2000, another proposal was made in Washington, D.C., to change the “-like viruses” to “-virus” resulting in today's Ebolavirus and Marburgvirus.

Each species of the genus Ebolavirus has one member virus, and four of these cause Ebola virus disease (EVD) in humans, characterized by having a very high case fatality rate; the fifth, Reston virus, has caused EVD in other primates. EBOV is the type species (reference or example species) for Ebolavirus, and has the highest mortality rate of the ebolaviruses, and is also responsible for the largest number of outbreaks of the five known members of the genus, including the 1976 EBOV outbreak and the 2013-2017 epidemic with the most deaths. The five characterized species of the Ebolavirus genus are:

Zaire ebolavirus (EBOV). Also known simply as the Zaire virus, EBOV has the highest case-fatality rate, up to 90% in some epidemics, with an average case fatality rate of approximately 83% over 27 years. There have been more outbreaks of EBOV than of any other species. The first outbreak took place on 26 Aug. 1976 in Yambuku. Mabalo Lokela, a 44-year-old schoolteacher, became the first recorded case. The symptoms resembled malaria, and subsequent patients received quinine. Transmission has been attributed to reuse of unsterilized needles and close personal contact. The virus is responsible for the 2014 West Africa EBOV outbreak, with the largest number of deaths to date.

Sudan ebolavirus (SUDV). Like EBOV, SUDV emerged in 1976; it was at first assumed to be identical with EBOV. SUDV is believed to have broken out first amongst cotton factory workers in Nzara, Sudan (now in South Sudan), in June 1976, with the first case reported as a worker exposed to a potential natural reservoir. Scientists tested local animals and insects in response to this; however, none tested positive for the virus. The carrier is still unknown. The lack of barrier nursing (or “bedside isolation”) facilitated the spread of the disease. The average fatality rates for SUDV were 54% in 1976, 68% in 1979, and 53% in 2000 and 2001.

Reston ebolavirus (RESTV). This virus was discovered during an outbreak of simian hemorrhagic fever virus (SHFV) in crab-eating macaques from Hazleton Laboratories (now Covance) in 1989. Since the initial outbreak in Reston, Virginia, it has since been found in nonhuman primates in Pennsylvania, Texas, and Siena, Italy. In each case, the affected animals had been imported from a facility in the Philippines, where the virus has also infected pigs. Despite its status as a Level-4 organism and its apparent pathogenicity in monkeys, RESTV did not cause disease in exposed human laboratory workers.

Tai Forest ebolavirus (TAFV). Formerly known as “Côte d'Ivoire ebolavirus,” it was first discovered among chimpanzees from the Tai Forest in Côte d'Ivoire, Africa, in 1994. Necropsies showed blood within the heart to be brown; no obvious marks were seen on the organs; and one necropsy displayed lungs filled with blood. Studies of tissues taken from the chimpanzees showed results similar to human cases during the 1976 EBOV outbreaks in Zaire and Sudan. As more dead chimpanzees were discovered, many tested positive for EBOV using molecular techniques. The source of the virus was believed to be the meat of infected western red colobus monkeys (Procolubus badius) upon which the chimpanzees preyed. One of the scientists performing the necropsies on the infected chimpanzees contracted the virus. She developed symptoms similar to those of dengue fever approximately a week after the necropsy, and was transported to Switzerland for treatment. She was discharged from hospital after two weeks and had fully recovered six weeks after the infection.

Bundibugyo ebolavirus (BDBV). On Nov. 24, 2007, the Uganda Ministry of Health confirmed an outbreak of ebolavirus in the Bundibugyo District. After confirmation of samples tested by the United States National Reference Laboratories and the CDC, the World Health Organization confirmed the presence of the new species. On 20 Feb. 2008, the Uganda Ministry officially announced the end of the epidemic in Bundibugyo, with the last infected person discharged on 8 Jan. 2008. An epidemiological study conducted by WHO and Uganda Ministry of Health scientists determined there were 116 confirmed and probable cases the new Ebola species, and that the outbreak had a mortality rate of 34% (39 deaths).

### B. Ebola Virus Disease

Symptoms of Ebola virus disease. The incubation period from infection with the virus to onset of symptoms is 2 to 21 days. Humans are not infectious until they develop symptoms. First symptoms are the sudden onset of fever fatigue, muscle pain, headache and sore throat. This is followed by vomiting, diarrhea, rash, symptoms of impaired kidney and liver function, and in some cases, both internal and external bleeding (e.g., oozing from the gums, blood in the stools). Laboratory findings include low white blood cell and platelet counts and elevated liver enzymes.

Diagnosis. It can be difficult to distinguish ebolavirus infections from other infectious diseases such as malaria, typhoid fever and meningitis. Confirmation that symptoms are caused by ebolaviruses infection are made using antibody-capture ELISA, antigen-capture detection tests, serum neutralization test, RT-PCR assay, electron microscopy, and virus isolation by cell culture. Samples from patients are an extreme biohazard risk; laboratory testing on non-inactivated samples should be conducted under maximum biological containment conditions.

Treatment and vaccines. Supportive care-rehydration with oral or intravenous fluids—and treatment of specific symptoms, improves survival. There is as yet no proven treatment available for ebolavirus. However, a range of potential treatments including blood products, immune therapies and drug therapies are currently being evaluated. No licensed vaccines are available yet, but 2 potential vaccines are undergoing human safety testing.

Prevention and control. Good outbreak control relies on applying a package of interventions, namely case management, surveillance and contact tracing, a good laboratory service, safe burials and social mobilization. Community engagement is key to successfully controlling outbreaks. Raising awareness of risk factors for ebolavirus infection and protective measures that individuals can take is an effective way to reduce human transmission. Risk reduction messaging should focus on several factors:
reducing the risk of wildlife-to-human transmission from contact with infected fruit bats or monkeys/apes and the consumption of their raw meat;
reducing the risk of human-to-human transmission from direct or close contact with people with Ebola symptoms, particularly with their bodily fluids;
outbreak containment measures including prompt and safe burial of the dead;
identifying people who may have been in contact with someone infected with Ebola and monitoring the health of contacts for 21 days;
the importance of separating the healthy from the sick to prevent further spread; and
the importance of good hygiene and maintaining a clean environment
   In terms of controlling infection in health-care settings, health-care workers should always take standard precautions when caring for patients, regardless of their presumed diagnosis. These include basic hand hygiene, respiratory hygiene, use of personal protective equipment (to block splashes or other contact with infected materials), safe injection practices and safe burial practices. Health-care workers caring for patients with suspected or confirmed Ebola virus should apply extra infection control measures to prevent contact with the patient's blood and body fluids and contaminated surfaces or materials such as clothing and bedding. When in close contact (within 1 meter) of patients with EBV, health-care workers should wear face protection (a face shield or a medical mask and goggles), a clean, non-sterile long-sleeved gown, and gloves (sterile gloves for some procedures). Laboratory workers are also at risk. Samples taken from humans and animals for investigation of Ebola infection should be handled by trained staff and processed in suitably equipped laboratories.

### II. MONOCLONAL ANTIBODIES AND PRODUCTION THEREOF

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

### A. General Methods

It will be understood that monoclonal antibodies binding to ebola virus will have several applications. These include the production of diagnostic kits for use in detecting and diagnosing ebola virus infection, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (mAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce ebola virus-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984). HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

mAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

### B. Antibodies of the Present Disclosure

Antibodies may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A. When the antibody neutralizes ebola virus, antibody escape mutant variant organisms can be isolated by propagating ebola virus in vitro or in animal models in the presence of high concentrations of the antibody. Sequence analysis of the ebola virus gene encoding the antigen targeted by the antibody reveals the mutation(s) conferring antibody escape, indicating residues in the epitope or that affect the structure of the epitope allosterically.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope, or may bind distinct epitopes. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference anti-ebola virus antibody, the above-described binding methodology is performed in two orientations: In a first orientation, the reference antibody is allowed to bind to the ebola virus antigen under saturating conditions followed by assessment of binding of the test antibody to the ebola virus molecule. In a second orientation, the test antibody is allowed to bind to the ebola virus antigen molecule under saturating conditions followed by assessment of binding of the reference antibody to the ebola virus molecule. If, in both orientations, only the first (saturating) antibody is capable of binding to the ebola virus, then it is concluded that the test antibody and the reference antibody compete for binding to the ebola virus. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, there is provided a mAb having the following variable region sequences:

| | | |
|---|---|---|
| [EBOV- | gaggtgcagttggtggagtctgggggaggcctcgtaaagccgggggggtcccttaga | SEQ ID NO: 1 |
| 442] | ctctcctgtacaggctctggaggcactggattcactttcaagtatgccgggatgagctg | |
| heavy | ggtccgccaggctccagggaaggggccggagtggattggccgtattaaaagcaggat | |
| | tgatggtgggacaacagactacgctgcacccgtgaaagacagattcattgtctcaaga | |
| | gatgattcaagaaatacactctatctgcaaatgaacagcctgaagaccgaggacaca | |
| | gccgtctattattgtgccacaggatcgggaaaggggccctctgcgtcgttcggggagtc | |
| | atactactactacgacttcattaacgtctggggcaaagggaccacggtcaccgtctcct | |
| | ca | |
| [EBOV- | gaaagtgtgttgacgcagtctccaggcaccctgtctttgtctccaggggaaagagcca | SEQ ID NO: 2 |
| 442] | ccctctcgtgcagggccagtcagagtattagcaggaagtacttagcctggtaccagca | |
| light | gaaacctggccaggctcccaggctcctcatctatggttcatccagcagggccactggc | |
| | atcccagacagattcagtggcagtgggtctgggacagacttcactctcaccatcagca | |
| | gactggagcctgaagattttgcagtgtattactgtcatcagtatgaaagctcaccttgg | |
| | acgttcggccaagggactaaggtggaaatcaaa | |
| [EBOV- | EVQLVESGGGLVKPGGSLRLSCTGSGGTGFTFKYAGMSWVRQAPG | SEQ ID NO: 3 |
| 442] | KGPEWIGRIKSRIDGGTTDYAAPVKDRFIVSRDDSRNTLYLQMNSLK | |
| heavy | TEDTAVYYCATGSGKGPSASFGESYYYYDFINVWGKGTTVTVSS | |
| [EBOV- | ESVLTQSPGTLSLSPGERATLSCRASQSISRKYLAWYQQKPGQAPRLL | SEQ ID NO: 4 |
| 442] | IYGSSSRATGIPDRFSGSGSGTDFTLTISRLEPEDFAVYYCHQYESSPW | |
| light | TFGQGTKVEIK | |

The heavy chain CDR1, CDR3 and CDR3 for EBOV-442 are GFTFKYAG (SEQ ID NO: 5), IKSRIDGGTT (SEQ ID NO: 6), and ATGSGKGPSASFGESYYYYDFINV (SEQ ID NO: 7), respectively, while the light chain CDR1, CDR2 and CDR3 for this antibody are QSISRKY (SEQ ID NO: 8), GSS (SEQ ID NO: 9), and HQYESSPWT (SEQ ID NO: 10), respectively.

In another aspect, there is provided a mAb having the following variable region sequences:

| | | |
|---|---|---|
| [EBOV-515] | caggtgcagctgcacgagtcgggcccaggactggtgaagccttcacagaccctgtccct | SEQ ID NO: 11 |
| heavy | cacctgcactgtctctggtgggtccatcaacagtgctggttactactggacctggatccg | |
| | ccagcacccggggaagggcctggagtggattgggtacatcgattatactgggaggacc | |
| | tactacaacccgtcccttgagagccgagtgatcatttcaatagacacgtctaagaacca | |
| | cttctccctgagactgacctctgtgtctgccgcggacacggccgtgtattactgtgcgcga | |
| | gaatcgtcgtgggtatccgagttagggcgtgacaactggggccagggaaccctggtca | |
| | ccgtctcttca | |
| [EBOV-515] | gaaatagtgatgacgcagtctccagccaccctgcctgtgtctccaggggaaggagccac | SEQ ID NO: 12 |
| light | cctctcctgcagggccagtcagagtgttttcaccaacttagcctggtaccagcaaaaacc | |
| | tggccaggctcccaggctcctcatctatgatgcatccaccagggccactggtatcccagc | |
| | caggttcagtggcagtgggtctgggacagagttcactctcaccatcagcagcctgcagt | |
| | ctgaagactttgcagtttattattgtcagcagtataataactggcctcggacgtacggcc | |
| | aagggaccagggtggaagtcaaa | |
| [EBOV-515] | QVQLHESGPGLVKPSQTLSLTCTVSGGSINSAGYYWTWIRQHPGKGL | SEQ ID NO: 13 |
| heavy | EWIGYIDYTGRTYYNPSLESRVIISIDTSKNHFSLRLTSVSAADTAVYYCA | |
| | RESSWVSELGRDNWGQGTLVTVSS | |
| [EBOV-515] | EIVMTQSPATLPVSPGEGATLSCRASQSVFTNLAWYQQKPGQAPRLL | SEQ ID NO: 14 |
| light | IYDASTRATGIPARFSGSGSGTEFTLTISSLQSEDFAVYYCQQYNNWPR | |
| | TYGQGTRVEVK | |

The heavy chain CDR1, CDR3 and CDR3 for EBOV-515 are GGSINSAGYY (SEQ ID NO: 15), IDYTGRT (SEQ ID NO: 16), and ARESSWVSELGRDN (SEQ ID NO: 17), respectively, while the light chain CDR1, CDR2 and CDR3 for this antibody are QSVFTN (SEQ ID NO: 18), DAS (SEQ ID NO: 19), and QQYNNWPRT (SEQ ID NO: 20), respectively.

When discussing variable sequence, which include additional “framework” regions, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C., to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below). Each of the foregoing applies to the variable region nucleic acid sequences and the amino acid sequences as set forth herein.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example, with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example, antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002), J. Biol. Chem. 277(30): 26733-26740; Davies J. et al. (2001), Biotechnology & Bioengineering 74(4): 288-294). Methods of altering carbohydrate contents are known to those skilled in the art, see, e.g., Wallick, S. C. et al. (1988), J. Exp. Med. 168(3): 1099-1109; Tao, M. H. et al. (1989), J. Immunol. 143(8): 2595-2601; Routledge, E. G. et al. (1995), Transplantation 60(8):847-53; Elliott, S. et al. (2003), Nature Biotechnol. 21:414-21; Shields, R. L. et al. (2002), J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

### C. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2a phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)2) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within +2 is preferred, those that are within +1 are particularly preferred, and those within +0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG, can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life (Kuo and Aveson, (2011)), including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat. In a further aspect of the disclosure the modifications are M252Y/S254T/T256E.

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH2 domain (according to the IMGT unique numbering for C-domain) were substituted with alanine residues. This modification, also known as “LALA” mutation, abolishes antibody binding to FcγRI, FcγRII and FcγRIIIa, as described by Hessell et al. (2007). The variant and unmodified recombinant mAbs were compared for their capacity to neutralize and enhance infection by the four dengue virus serotypes. LALA variants retained the same neutralizing activity as unmodified mAb but were completely devoid of enhancing activity. LALA mutations of this nature are therefore contemplated in the context of the presently disclosed antibodies.

A Pro329Gly mutation can added to the LALA mutations, which inhibits binding to murine FcγRI, II, and III by IgG2a Fc (Sauders, Frontiers in Immunology, 10, article 1296, 2013). The amino acid at 329 makes contact with Trp108 and Trp131 of FcγRIIIa. The LALA-PG is thus an improvement over LALA mutations alone in that it nullified Fc function in mouse and human IgG, whereas LALA alone still retains murine FcγRIII binding to murine IgG2a. The significance of the LALA-PG mutations are that observed results in murine models are expected to more accurately translate to humans since the mutations confer a similar phenotype for both murine IgG2a and human IgG1. Also, the LALA-PG mutation may afford more profound Fc-knockout and may be preferred.

Altered Glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, GIF, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication 20030003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG1, IgG2, IgG3, and IgG4 subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection, however it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

### D. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### E. Multispecific Antibodies

In certain embodiments, antibodies of the present disclosure are bispecific or multispecific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an anti-pathogen arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess a pathogen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). WO 96/16673 describes a bispecific anti-ErbB2/anti-Fc gamma RIII antibody and U.S. Pat. No. 5,837,234 discloses a bispecific anti-ErbB2/anti-Fc gamma RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full-length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998). doi: 10.1038/nbt0798-677pmid:9661204). For example, bispecific antibodies have been produced using leucine zippers (Kostelny et al., J. Immunol., 148(5): 1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

In a particular embodiment, a bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004: 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). A multivalent antibody may be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1),-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

Accordingly, in particular embodiments, an antibody comprised in the therapeutic agent comprises
(a) a first Fab molecule which specifically binds to a first antigen
(b) a second Fab molecule which specifically binds to a second antigen, and wherein the variable domains VL and VH of the Fab light chain and the Fab heavy chain are replaced by each other,
wherein the first antigen is an activating T cell antigen and the second antigen is a target cell antigen, or the first antigen is a target cell antigen and the second antigen is an activating T cell antigen; and
wherein
i) in the constant domain CL of the first Fab molecule under a) the amino acid at position 124 is substituted by a positively charged amino acid (numbering according to Kabat), and wherein in the constant domain CHI of the first Fab molecule under a) the amino acid at position 147 or the amino acid at position 213 is substituted by a negatively charged amino acid (numbering according to Kabat EU index); or
ii) in the constant domain CL of the second Fab molecule under b) the amino acid at position 124 is substituted by a positively charged amino acid (numbering according to Kabat), and wherein in the constant domain CHI of the second Fab molecule under b) the amino acid at position 147 or the amino acid at position 213 is substituted by a negatively charged amino acid (numbering according to Kabat EU index).

The antibody may not comprise both modifications mentioned under i) and ii). The constant domains CL and CHI of the second Fab molecule are not replaced by each other (i.e., remain unexchanged).

In another embodiment of the antibody, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)), and in the constant domain CHI of the first Fab molecule under a) the amino acid at position 147 or the amino acid at position 213 is substituted independently by glutamic acid (E), or aspartic acid (D) (numbering according to Kabat EU index).

In a further embodiment, in the constant domain CL of the first Fab molecule under a) the amino acid at position 124 is substituted independently by lysine (K), arginine (R) or histidine (H) (numbering according to Kabat), and in the constant domain CHI of the first Fab molecule under a) the amino acid at position 147 is substituted independently by glutamic acid (E), or aspartic acid (D) (numbering according to Kabat EU index).

In a particular embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CHI of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more particular embodiment, in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and in the constant domain CHI of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In an even more particular embodiment, in the constant domain CL of the first Fab molecule under a) the amino acid at position 124 is substituted by lysine (K) (numbering according to Kabat) and the amino acid at position 123 is substituted by arginine (R) (numbering according to Kabat), and in the constant domain CHI of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

### F. Chimeric Antigen Receptors

Artificial T cell receptors (also known as chimeric T cell receptors, chimeric immunoreceptors, chimeric antigen receptors (CARs)) are engineered receptors, which graft an arbitrary specificity onto an immune effector cell. Typically, these receptors are used to graft the specificity of a monoclonal antibody onto a T cell, with transfer of their coding sequence facilitated by retroviral vectors. In this way, a large number of target-specific T cells can be generated for adoptive cell transfer. Phase I clinical studies of this approach show efficacy.

The most common form of these molecules are fusions of single-chain variable fragments (scFv) derived from monoclonal antibodies, fused to CD3-zeta transmembrane and endodomain. Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g., neuroblastoma cells). To target malignant B cells, investigators have redirected the specificity of T cells using a chimeric immunoreceptor specific for the B-lineage molecule, CD19.

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows to the scFv to orient in different directions to enable antigen binding. The transmembrane domain is a typical hydrophobic alpha helix usually derived from the original molecule of the signaling endodomain which protrudes into the cell and transmits the desired signal.

Type I proteins are in fact two protein domains linked by a transmembrane alpha helix in between. The cell membrane lipid bilayer, through which the transmembrane domain passes, acts to isolate the inside portion (endodomain) from the external portion (ectodomain). It is not so surprising that attaching an ectodomain from one protein to an endodomain of another protein results in a molecule that combines the recognition of the former to the signal of the latter.

Ectodomain. A signal peptide directs the nascent protein into the endoplasmic reticulum. This is essential if the receptor is to be glycosylated and anchored in the cell membrane. Any eukaryotic signal peptide sequence usually works fine. Generally, the signal peptide natively attached to the amino-terminal most component is used (e.g., in a scFv with orientation light chain-linker-heavy chain, the native signal of the light-chain is used

The antigen recognition domain is usually an scFv. There are however many alternatives. An antigen recognition domain from native T-cell receptor (TCR) alpha and beta single chains have been described, as have simple ectodomains (e.g., CD4 ectodomain to recognize HIV infected cells) and more exotic recognition components such as a linked cytokine (which leads to recognition of cells bearing the cytokine receptor). In fact, almost anything that binds a given target with high affinity can be used as an antigen recognition region.

A spacer region links the antigen binding domain to the transmembrane domain. It should be flexible enough to allow the antigen binding domain to orient in different directions to facilitate antigen recognition. The simplest form is the hinge region from IgG1. Alternatives include the CH₂CH₃ region of immunoglobulin and portions of CD3. For most scFv based constructs, the IgG1 hinge suffices. However, the best spacer often has to be determined empirically.

Transmembrane domain. The transmembrane domain is a hydrophobic alpha helix that spans the membrane. Generally, the transmembrane domain from the most membrane proximal component of the endodomain is used. Interestingly, using the CD3-zeta transmembrane domain may result in incorporation of the artificial TCR into the native TCR a factor that is dependent on the presence of the native CD3-zeta transmembrane charged aspartic acid residue. Different transmembrane domains result in different receptor stability. The CD28 transmembrane domain results in a brightly expressed, stable receptor.

Endodomain. This is the “business-end” of the receptor. After antigen recognition, receptors cluster and a signal is transmitted to the cell. The most commonly used endodomain component is CD3-zeta which contains 3 ITAMs. This transmits an activation signal to the T cell after antigen is bound. CD3-zeta may not provide a fully competent activation signal and additional co-stimulatory signaling is needed.

“First-generation” CARs typically had the intracellular domain from the CD3 g-chain, which is the primary transmitter of signals from endogenous TCRs. “Second-generation” CARs add intracellular signaling domains from various costimulatory protein receptors (e.g., CD28, 41BB, ICOS) to the cytoplasmic tail of the CAR to provide additional signals to the T cell. Preclinical studies have indicated that the second generation of CAR designs improves the antitumor activity of T cells. More recent, “third-generation” CARs combine multiple signaling domains, such as CD3z-CD28-41BB or CD3z-CD28-OX40, to further augment potency.

### G. ADCs

Antibody Drug Conjugates or ADCs are a new class of highly potent biopharmaceutical drugs designed as a targeted therapy for the treatment of people with infectious disease. ADCs are complex molecules composed of an antibody (a whole mAb or an antibody fragment such as a single-chain variable fragment, or scFv) linked, via a stable chemical linker with labile bonds, to a biological active cytotoxic/anti-viral payload or drug. Antibody Drug Conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the infected cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on infected cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cell or impairs viral replication. Due to this targeting, ideally the drug has lower side effects and gives a wider therapeutic window than other agents.

A stable link between the antibody and cytotoxic/anti-viral agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (anti-cancer) drug. For example, a non-cleavable linker keeps the drug within the cell. As a result, the entire antibody, linker and cytotoxic agent enter the targeted cancer cell where the antibody is degraded to the level of an amino acid. The resulting complex-amino acid, linker and cytotoxic agent-now becomes the active drug. In contrast, cleavable linkers are catalyzed by enzymes in the host cell where it releases the cytotoxic agent.

Another type of cleavable linker, currently in development, adds an extra molecule between the cytotoxic/anti-viral drug and the cleavage site. This linker technology allows researchers to create ADCs with more flexibility without worrying about changing cleavage kinetics. Researchers are also developing a new method of peptide cleavage based on Edman degradation, a method of sequencing amino acids in a peptide. Future direction in the development of ADCs also include the development of site-specific conjugation (TDCs) to further improve stability and therapeutic index and a emitting immunoconjugates and antibody-conjugated nanoparticles.

### H. BiTES

Bi-specific T-cell engagers (BiTEs) are a class of artificial bispecific monoclonal antibodies that are investigated for the use as anti-cancer drugs. They direct a host's immune system, more specifically the T cells' cytotoxic activity, against infected cells. BiTE is a registered trademark of Micromet AG.

BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic/anti-viral activity on infected cells by producing proteins like perforin and granzymes, independently of the presence of MHC I or co-stimulatory molecules. These proteins enter infected cells and initiate the cell's apoptosis. This action mimics physiological processes observed during T cell attacks against infected cells.

### I. Intrabodies

In a particular embodiment, the antibody is a recombinant antibody that is suitable for action inside of a cell-such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

By virtue of their ability to enter cells, intrabodies have additional uses that other types of antibodies may not achieve. In the case of the present antibodies, the ability to interact with the MUCI cytoplasmic domain in a living cell may interfere with functions associated with the MUC1 CD, such as signaling functions (binding to other molecules) or oligomer formation. In particular, it is contemplated that such antibodies can be used to inhibit MUCI dimer formation.

### J. Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. ACTIVE/PASSIVE IMMUNIZATION AND TREATMENT/PREVENTION OF EBOLA VIRUS INFECTION

### A. Formulation and Administration

The present disclosure provides pharmaceutical compositions comprising anti-ebola virus antibodies and antigens for generating the same. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Active vaccines are also envisioned where antibodies like those disclosed are produced in vivo in a subject at risk of ebola virus infection. Such vaccines can be formulated for parenteral administration, e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example, by nasal drops, inhalation, by nebulizer, or via intrarectal or vaginal delivery. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### 2. ADCC

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. By “antibody having increased/reduced antibody dependent cell-mediated cytotoxicity (ADCC)” is meant an antibody having increased/reduced ADCC as determined by any suitable method known to those of ordinary skill in the art.

As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example, the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

### 3. CDC

Complement-dependent cytotoxicity (CDC) is a function of the complement system. It is the processes in the immune system that kill pathogens by damaging their membranes without the involvement of antibodies or cells of the immune system. There are three main processes. All three insert one or more membrane attack complexes (MAC) into the pathogen which cause lethal colloid-osmotic swelling, i.e., CDC. It is one of the mechanisms by which antibodies or antibody fragments have an anti-viral effect.

### IV. ANTIBODY CONJUGATES

Antibodies of the present disclosure may be linked to at least one therapeutic agent to form an antibody conjugate. In order to increase the efficacy of antibody molecules as therapeutic agents, it is conventional to link or covalently bind or complex at least one therapeutic molecule or moiety. Such a molecule or moiety may be, but is not limited to, at least one effector or reporter molecule.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3a-6a-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### V. Kits

In still further embodiments, the present disclosure concerns kits for use with the antibodies and methods described above. Further suitable reagents for use in the present kits include buffers and diluents as well as instruments for administration. The kit may also contain instructions for use. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody and any other reagent containers in close confinement for commercial sale. Such containers may include injection or blow-molded plastic containers into which the desired vials are retained.

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1-Materials and Methods

Cell lines. Vero-E6 (monkey, female origin) and Vero CCL-81 (monkey, female origin) were obtained from the American Type Culture Collection (ATCC). Vero-E6 cells were cultured in Minimal Essential Medium (MEM) (Thermo Fisher Scientific) supplemented with 10% fetal bovine serum (FBS; HyClone) and 1% penicillin-streptomycin in 5% CO₂, 37° C. Vero CCL-81 cells were cultured in Dulbecco's Modified Eagle Medium (DMEM; Thermo Fisher Scientific) supplemented with 10% Ultra-Low IgG FBS (Gibco), 25 mM HEPES, and 100 units/mL of penicillin, and 100 μg/mL of streptomycin (GIBCO) in 5% CO₂, 37° C. A 293F cell line (human, female origin) stably-transfected to express SNAP-tagged EBOV GP was described previously (Domi et al., 2018). ExpiCHO (hamster, female origin) and FreeStyle 293F (human, female origin) cell lines were purchased from Thermo Fisher Scientific and cultured according to the manufacturer's protocol. The Jurkat-EBOV GP (Makona variant) cell line stably transduced to display EBOV GP on the surface (Davis et al., 2019) was a kind gift from Carl Davis (Emory University, Atlanta, GA). All cell lines were tested on a monthly basis for Mycoplasma and found to be negative in all cases.

Viruses. The mouse-adapted EBOV Mayinga variant (EBOV-MA, GenBank: AF49101) (Bray et al., 1998), authentic EBOV Mayinga variant expressing eGFP (Towner et al., 2005), the chimeric infectious EBOV/BDBV-GP (GenBank: KU174137) and EBOV/SUDV-GP (GenBank: KU174142) viruses expressing eGFP (Ilinykh et al., 2016), the infectious vesicular stomatitis viruses rVSV/EBOV GP (Mayinga variant) (Garbutt et al., 2004), rVSV/BDBV GP (Uganda variant) (Mire et al., 2013), or rVSV/SUDV GP (Boniface variant) (Geisbert et al., 2008) expressing an ebolavirus GP that replaces VSV G protein were used for mouse challenge studies or neutralization assays. Viruses were grown and titrated in Vero cell monolayer cultures.

Authentic ebolaviruses EBOV (Cross et al., 2016), BDBV (Towner et al., 2008), and SUDV (Thi et al., 2016) were used for NHP challenge studies. The EBOV isolate 199510621 (Kikwit variant) originated from a 65-year-old female patient who died on 5 May 1995. The study challenge material was from the second Vero-E6 passage of EBOV isolate 199510621. The first passage at UTMB consisted of inoculating CDC 807223 (passage 1 of EBOV isolate 199510621) at a MOI of 0.001 onto Vero E6 cells. SUDV isolate 200011676 (variant Gulu) originated from a 35-year-old male patient who died on 16 Oct. 2000. The study challenge material was from the second Vero-E6 cell passage of SUDV isolate 200011676. The first passage at UTMB consisted of inoculating CDC 808892 (CDC passage 1 of SUDV isolate 200011676) at a MOI of 0.001 onto Vero-E6 cells. BDBV isolate 200706291 variant Uganda originated from serum of a patient collected in Uganda on 1 Oct. 2007. The study challenge material was from the second Vero-E6 cell passage of BDBV isolate 200706291 variant Uganda. Briefly, the first passage at UTMB consisted of inoculating CDC 811250 (CDC passage 1 of BDBV isolate 200706291) at a MOI of 0.001 onto Vero-E6 cells. The cell culture fluids were subsequently harvested at day 10 post inoculation with each of indicated viruses and stored at −80° C. as ˜1 mL aliquots. Neither mycoplasma nor endotoxin were detected (<0.5 endotoxin units (EU)/mL).

Mouse model. Seven-to eight-week old female BALB/c mice were obtained from the Jackson Laboratory. Mice were housed in microisolator cages and provided food and water ad libitum. Challenge studies were conducted under maximum containment in an animal biosafety level 4 (ABSL-4) facility of the Galveston National Laboratory, UTMB. The animal protocols for testing of mAbs in mice were approved by the Institutional Animal Care and Use Committee of the University of Texas Medical Branch (UTMB) in compliance with the Animal Welfare Act and other applicable federal statutes and regulations relating to animals and experiments involving animals.

Nonhuman primate (NHP) model. Three-to four-year-old male (n=6) or female (n=6) rhesus macaques used in this study were obtained from PrimGen. Four-year-old male (n=3) or female (n=3) cynomolgus monkeys were obtained from Worldwide Primates. NHP research adhered to principles stated in the eighth edition of the Guide for the Care and Use of Laboratory Animals (National Research Council, 2011). The facility where this research was conducted [University of Texas Medical Branch (UTMB)] is fully accredited by the Association for Assessment and Accreditation of Laboratory Animal Care International and has an approved Office of Laboratory Animal Welfare Assurance (#A3314-01).

Monoclonal antibody production and purification. Sequences of monoclonal antibodies that had been synthesized as cDNA (Twist Bioscience) and cloned into an IgG1 or IgG1 LALA-PG monocistronic expression vector (designated as pTwist-mCis_G1 or pTwist-mCis_hG1 LALA-PG) were used for monoclonal antibody secretion in mammalian cell culture. This vector contains an enhanced 2A sequence and GSG linker that allows the simultaneous expression of monoclonal antibody heavy and light chain genes from a single construct upon transfection (Chng et al., 2015). CHO cell cultures were transfected using the Gibco ExpiCHO Expression System protocols as described by the vendor. Culture supernatants were purified using 5 mL HiTrap MabSelect SuRe (Cytiva, formerly GE Healthcare Life Sciences) column and an ÄKTA pure chromatography system (Cytiva). Purified monoclonal antibodies were buffer-exchanged into PBS, concentrated using Amicon Ultra-4 50-kDa centrifugal filter units (Millipore Sigma) and stored at 4° C. until use. For NHP treatment studies antibodies were purified from 5 to 15 L of CHO supernatant using HiScale 26/20 column (Cytiva) packed with MabSelect SuRe resin, purified protein was buffer-exchanged into PBS using HiScale 50/40 column packed with Sephadex G-25 (medium) resin (GE Healthcare Life Sciences), concentrated, and stored at −80° C. until use. Purified monoclonal antibodies were tested routinely for endotoxin levels (found to be less than 30 EU per mg IgG for mouse studies and less than 1 EU per mg IgG for NHP studies). Endotoxin testing was performed using the PTS201F cartridge (Charles River), with a sensitivity range from 10 to 0.1 EU per mL, and an Endosafe Nexgen-MCS instrument (Charles River). For structural studies, Fab was produced after co-transfection of ExpiCHO cells with two separate mammalian expression vectors containing antibody light chain and Fab heavy chain sequences as described previously (Gilchuk et al., 2020b). Fab proteins were purified using CaptureSelect column (Thermo Fisher Scientific). Purified antibodies were buffer exchanged into PBS, concentrated using Amicon Ultra-4 30 kDa MWCO centrifugal filter units (Millipore Sigma) and stored at 4° C. until use.

GP expression and purification. For ELISA studies, the ectodomains of EBOV GP ΔTM (residues 1-636; strain Makona; GenBank: KM233070), BDBV GP ΔTM (residues 1-643; strain 200706291 Uganda; GenBank: NC_014373), SUDV GP ΔTM (residues 1-637; strain Gulu; GenBank: NC_006432), and MARV GP ΔTM (residues 1-648; strain Angola2005; GenBank: DQ447653) were expressed and purified as described before (Gilchuk et al., 2018a). For structural studies, the ectodomain of EBOV/Makona GP (residues 32-644, GenBank AKG65268.1) lacking residues 310-460 of the mucin-like domain to produce EBOV/Makona GPΔMuc was produced and purified as described before (Murin et al., 2021).

ELISA binding assay. To assess mAb binding at different pH, wells of 96-well microtiter plates were coated with purified, recombinant EBOV, BDBV or SUDV GPA™ ectodomains at 4° C., overnight. Plates were blocked with 2% non-fat dry milk and 2% normal goat serum in DPBS containing 0.05% Tween-20 (DPBS-T) for 1 h. Purified mAbs were diluted serially in DPBS-T (pH 7.4), or DPBS-T that was adjusted to pH 5.5 or 4.5 with hydrochloric acid, added to the wells and incubated for 1 h at ambient temperature. The bound antibodies were detected using goat anti-human IgG conjugated with horseradish peroxidase (Southern Biotech) diluted in blocking buffer. Color development was monitored using TMB (3,3′,5,5′-tetramethylbenzidine) substrate (Thermo Fisher Scientific), IN hydrochloric acid was added to stop the reaction, and the absorbance was measured at 450 nm using a spectrophotometer (Biotek).

Mammalian cell-surface-displayed GP antibody binding. Binding of Alexa Fluor 647-labeled antibody to Jurkat-EBOV GP cell line was assessed by flow cytometry using an iQue Screener Plus high throughput flow cytometer (Intellicyt Corp.) as the inventors described previously (Gilchuk et al., 2020b).

Measurement of synergistic GP binding by a combination of antibodies. Serially-diluted Alexa Fluor 647-labeled antibody rEBOV-442 IgG1 or rEBOV-515 LALA-PG was titrated into serially-diluted unlabeled partner antibody to generate a pairwise combinatorial matrix of two antibodies in the mixture. For antibody dilutions and washes, the inventors used DPBS (Dulbecco's phosphate-buffered saline) containing 2% of heat-inactivated FBS and 2 mM EDTA (ethylenediaminetetraacetic acid, sodium salt) (pH 8.0) designated as incubation buffer. For antibody staining, ˜5×10⁴ Jurkat EBOV-GP cells were added per each well of V-bottom 96-well plate (Corning) in 5 μL of the incubation buffer, and antibody mixtures were added to the cells in duplicate for total volume of 50 μL per well, followed by 2 hr incubation at 4° C. Cells were washed with the incubation buffer by centrifugation at 400×g for 5 min at ambient temperature and binding to the GP was assessed using iQue Screener Plus flow cytometer. Data for up to 5,000 events per well were acquired, and data were analyzed with ForeCyt (Intellicyt Corp.) software. Dead cells were excluded from the analysis on the basis of forward and side scatter gate for viable cell population. Binding was calculated as the percent of the maximal median fluorescence intensity signal (MFI) by the highest concentration of respective fluorescently-labeled antibody alone (25 μg/mL). Synergy distribution maps were generated from the dose-response binding matrix using a web application, SynergyFinder 2.0, and data was analyzed using ZIP synergy scoring model (Ianevski et al., 2020).

Selection and sequencing of VSV/EBOV GP mutants that escape antibody neutralization. To screen for escape mutations selected in the presence of individual antibodies or antibody cocktails, the inventors used a real-time cell analysis (RTCA) assay and xCELLigence RTCA MP Analyzer (ACEA Biosciences Inc.) with modification of recently described assays (Gilchuk et al., 2020a; Greaney et al., 2021). Fifty (50) μL of cell culture medium (DMEM supplemented with 2% FBS) was added to each well of a 96-well E-plate to obtain a background reading. Eighteen thousand (18,000) Vero cells in 50 μL of cell culture medium were seeded per each well, and plates were placed on the analyzer. Measurements were taken automatically every 15 min and the sensograms were visualized using RTCA software version 2.1.0 (ACEA Biosciences Inc). VSV/EBOV GP virus (20,000 plaque forming units [PFU] per well, ˜1 MOI) was mixed with a saturating neutralizing concentration of individual antibody (10 μg/mL) or two-antibody cocktail (1:1 antibody ratio, 10 μg/mL total antibody concentration) in a total volume of 100 μL and incubated for 1 h at 37° C. At 16-20 h after seeding the cells, the virus-antibody mixtures were added into 8 to 96 replicate wells of 96-well E-plates with cell monolayers. Wells containing only virus in the absence of antibody and wells containing only Vero cells in medium were included on each plate as controls. Plates were measured continuously (every 15 min) for 72 h. The escape mutants were identified by delayed CPE in wells containing antibody. To verify escape from rEBOV-442 antibody selection, isolated viruses were assessed in a subsequent RTCA experiment in the presence of 20 μg/mL of rEBOV-442 or 20 μg/mL of rEBOV-515 or 20 μg/mL of 1:1 cocktail of rEBOV-442+rEBOV-515 (see FIG. S1B).

To verify escape mutations present in GP protein-expressing VSV antibody-selected escape variants, the escape viruses isolated after RTCA escape screening were propagated in 6-well culture plates with confluent Vero cells in the presence of 20 μg/mL of the rEBOV-442. Viral RNA was isolated using a QiAmp Viral RNA extraction kit (QIAGEN) from aliquots of supernatant containing a suspension of the selected virus population. The GP protein gene cDNA was amplified with a SuperScript IV One-Step RT-PCR kit (Thermo Fisher Scientific) using primers flanking the GP gene. The amplified PCR product (˜2,400 bp) was purified using SPRI magnetic beads (Beckman Coulter) at a 1:1 ratio and sequenced by the Sanger sequence technique using primers giving forward and reverse reads of the glycan cap region of the GP.

Neutralization assays. BSL-4 virus neutralization assays were performed using recombinant EBOV-eGFP or chimeric EBOV viruses in which GP was replaced with its counterpart from BDBV or SUDV, as described previously (Ilinykh et al., 2016). Briefly, four-fold dilutions of the respective mAb starting at 200 μg/mL were mixed in triplicate with 400 PFU of the virus in U-bottom 96-well plates and incubated for 1 hr at 37° C. Mixtures were applied on Vero-E6 cell monolayer cultures in 96-well plates and incubated for four days at 37° C. In the absence of mAb neutralizing activity, the infection resulted in uniform eGFP fluorescence from the monolayer of cells that was detected readily by fluorescence microscopy. Fluorescence was measured using Synergy HT microplate reader (BioTek). Half maximal inhibitory concentration (ICso) values were determined by nonlinear regression analysis using Prism software.

BSL-2 virus neutralization experiments were performed using the infectious rVSV/EBOV GP, rVSV/BDBV GP, and rVSV/SUDV GP viruses, and the inventors adopted high-throughput RTCA assay that quantify virus-induced cytopathic effect (CPE) (Gilchuk et al., 2020a; Gilchuk et al., 2020b). Viruses were pre-titrated by RTCA to determine dilution of each virus stock to achieve similar CPE kinetics and complete CPE in 32 h after applying virus alone to Vero cells. Fifty (50) μL of cell culture medium (DMEM supplemented with 2% FBS) was added to each well of a 96-well E-plate using a ViaFlo384 liquid handler (Integra Biosciences) to obtain background reading. Eighteen thousand (18,000) Vero cells in 50 μL of cell culture medium were seeded per each well, and the plate was placed on the analyzer. Measurements were taken automatically every 15 min, and the sensograms were visualized using RTCA software version 2.1.0 (ACEA Biosciences Inc). VSV/EBOV GP (˜0.1 MOI, ˜2,000 PFU per well), or VSV/BDBV GP (0.04 MOI, ˜800 PFU per well) or VSV/SUDV GP (0.01 MOI, ˜240 PFU per well) were mixed 1:1 with respective dilution of mAb in triplicate a total volume of 100 μL using DMEM supplemented with 2% FBS as a diluent and incubated for 1 h at 37° C. in 5% CO₂. At 16 to 18 h after seeding the cells, the virus-mAb mixtures were added to the cells in 96-well E-plates. Triplicate wells containing virus only (maximal CPE in the absence of mAb) and wells containing only Vero cells in medium (no-CPE wells) were included as controls. Plates were measured continuously (every 15 min) for 48 h to assess virus neutralization. Normalized cellular index (CI) values at the endpoint (42 h after incubation with the virus) were determined using the RTCA software version 2.1.0 (ACEA Biosciences Inc.). Results were expressed as percent neutralization in the presence of a particular mAb relative to no-CPE control wells minus CI values from control wells with maximum CPE. RTCA IC₅₀ values were determined by nonlinear regression analysis using Prism software.

Measurement of synergistic virus neutralization by a combination of antibodies. The inventors used RTCA assay to assess neutralizing activity from a pairwise combinatorial matrix of two antibodies in the mixture. Serially diluted rEBOV-442 (2-fold dilutions) was titrated into serially-diluted rEBOV-515 (two-fold dilutions) and incubated with rVSV/EBOV GP, rVSV/BDBV GP, or rVSV/SUDV GP viruses for 1 h at 37° C. Virus-antibody mixtures were applied to Vero cells grown in 96-well E-plates in duplicates for each virus and using separate plates for each pairwise combinatorial matrix. Triplicate wells containing virus only (maximal CPE in the absence of mAb) and wells containing only Vero cells in medium (no CPE wells) were included as controls. Plates were measured continuously (every 15 minutes) for 48 h to assess virus neutralization. Normalized cellular index (CI) values at the endpoint (42 h after incubation with the virus) were determined using the RTCA software version 2.1.0 (ACEA Biosciences Inc.). Results were expressed as percent neutralization in the presence of a particular mAb relative to control wells with no CPE minus CI values from control wells with maximum CPE. Synergy distribution maps were generated from the dose-response binding matrix using a web application, SynergyFinder 2.0, and data was analyzed using ZIP synergy scoring model (Ianevski et al., 2020).

Rapid fluorometric antibody-mediated cytotoxicity assay (RFADCC). Antibody-dependent cell-mediated cytotoxicity activity of EBOV GP-reactive IgG was quantified with an EBOV-adapted modification of the RFADCC assay (Domi et al., 2018; Orlandi et al., 2016). Briefly, a target cell line was made by transfecting 293F cells with a full-length DNA expressing GP from the EBOV-Kikwit isolate followed by transfecting with two separate DNA constructs expressing eGFP and the chimeric CCR5-SNAP tag protein. The new cell line, designated EBOV GPkik-293FS eGFP CCR5-SNAP, expresses EBOV-Kikwit GP on the plasma membrane, eGFP in the cytoplasm and the SNAP-tag CCR5, which can be specifically labeled with SNAP-Surface AF647 (NEB), on the cell surface (Domi et al., 2018). The unrelated human mAb DENV 2D22 and the Fc effector function disabled mAb rEBOV-515 LALA-PG were used as negative controls for the assay background. The ADCC activity was quantified by incubating three-fold serial dilutions of mAbs with EBOV GPkik-293FS eGFP CCR5-SNAP target cells for 15 min at ambient temperature and then adding human PBMC as effector cells for 2 hrs at 37° C., after which cells were washed once with PBS, fixed with 2% PFA, stained and analyzed using an iQue Screener Plus flow cytometer (Intellicyt Corp.). Data analysis was performed with ForeCyt (Intellicyt Corp.) software. The percentage cytotoxicity of the mAb was determined as the number of target cells losing eGFP signal (by virtue of ADCC) but retaining the surface expression of CCR5-SNAP.

GP cleavage inhibition. The assay was performed as the inventors described previously (Gilchuk et al., 2018a). Briefly, Jurkat-EBOV GP cells were pre-incubated with serial dilutions of mAbs in DPBS for 20 min at room temperature, then incubated with thermolysin (Promega) diluted in DPBS to 1 mg/mL for 20 min at 37° C. The reaction was stopped by addition of the incubation buffer containing DPBS, 2% heat-inactivated FBS and 2 mM EDTA (pH 8.0). Washed cells were incubated with 5 μg/mL of Alexa Fluor 647-labeled EBOV GP RBD-reactive mAb MR78 (Bornholdt et al., 2016) at 4° C., for 60 min. Stained cells were washed, fixed, and analyzed by flow cytometry using Intellicyt iQue. Cells were gated for the viable population, and median fluorescence intensity from Alexa Fluor 647 was determined. Background staining was determined from binding of the labeled mAb MR78 to Jurkat-EBOV GP (uncleaved) cells. Results are expressed as the percent of RBS exposure inhibition in the presence of tested mAb relative to controls for minimal binding of labeled MR78 mAb-only to intact (uncleaved) Jurkat EBOV-GP, and maximal binding of labeled MR78 mAb-only to cleaved Jurkat-EBOV GP.

Mouse challenge. Groups of mice (n=5 per group) were inoculated with 1,000 PFU of the EBOV-MA by the intraperitoneal (i.p.) route. Mice were treated i.p. with indicated doses of individual mAbs on 1 day after virus inoculation (dpi). Human mAb DENV 2D22 served as a control. Mice were monitored twice daily from 0 to 14 dpi for illness, survival, and weight loss, followed by once daily monitoring from 15 dpi to the end of the study at 28 dpi. The extent of disease was scored using the following parameters: score 1-healthy; score 2-ruffled fur and hunched posture; score 3-a score of 2 plus one additional clinical sign such as orbital tightening and/or >15% weight loss; score 4-a score of 3 plus one additional clinical sign such as reluctance to move when stimulated, or any neurologic signs (seizures, tremors, head tilt, paralysis, etc.), or >20% weight loss. Animals reaching a score of 4 were euthanized as per the IACUC-approved protocol. All mice were euthanized on day 28 after EBOV challenge.

NHP challenge. Twelve healthy adult rhesus macaques (Macaca mulatta) of Chinese origin and six healthy adult cynomolgus monkeys (Macaca fascicularis) were studied. Animals were assigned to three groups of five animals per treatment group and a control untreated animal. Animals were randomized by random number assignment (with Microsoft Excel) into a treatment group and a control animal. After intramuscular challenge with a lethal target dose of 1,000 plaque-forming units (PFU) of EBOV/Kikwit, BDBV/Uganda, or SUDV/Gulu, each of the NHPs of the treatment group received intravenously two 30 mg/kg doses of the cocktail (a 2:1 mixture of rEBOV-515 LALA-PG and rEBOV-442 IgG1) spaced 3 days apart (days 3 and 6 after EBOV/Kikwit, days 6 and 9 after BDBV/Uganda, or days 4 and 7 after SUDV SUDV/Gulu inoculation). The back-titer of the EBOV, BDBV, and SUDV inoculum identified 963, 1113, and 988 PFU as the actual inoculation dose for the respective virus. Historical untreated controls for EBOV challenge cohort included fourteen untreated animals from separate studies including 11 animals, as the inventors reported previously (Gilchuk et al., 2020b), which were challenged with the same target dose of EBOV/Kikwit and by the same route. Historical untreated controls for SUDV/Gulu challenge cohort included five untreated animals from previous study (Thi et al., 2016) and four untreated animals from two other studies (Geisbert and Cross, unpublished) that were challenged with the same target dose of BDBV and by the same route. Historical untreated controls for BDBV challenge cohort included three untreated animals from a previous study (Bornholdt et al., 2019) and seventeen untreated animals from the other studies (Geisbert and Cross, unpublished) that were challenged with the same target dose of BDBV/Uganda and by the same route. All animals were given physical exams, and blood was collected at the time of inoculation and at indicated times after virus inoculation. In addition, all animals were monitored daily and scored for disease progression with an internal filovirus scoring protocol approved by the UTMB Institutional Animal Care and Use Committee. The scoring measured from baseline and included posture or activity level, attitude or behavior, food and water intake, respiration, and disease manifestations such as visible rash, hemorrhage, ecchymosis, or flushed skin. A score of ≥9 indicated that an animal met criteria for euthanasia. These studies were not blinded, and all animals were included in analysis.

Measurement of virus load in NHP blood and tissues. Titration of virus in plasma samples and 10% tissue homogenates (w/v) was performed by plaque assay in Vero-E6 cell culture monolayers. Briefly, serial 10-fold dilutions of the samples were applied to Vero-E6 cell monolayers in duplicate wells (200 μL); the limit of detection was 25 PFU/mL for plasma and 250 PFU/gram for tissue. For qRT-PCR analysis, RNA was isolated from whole blood or tissue using the Viral RNA Mini-kit (Qiagen) using 100 μL of blood or 100 mg of tissue into 600 μL of buffer AVL. Primers (probes) targeting the VP30 gene of EBOV probe sequence of 6-carboxyflourescein (6FAM)-5′ CCG TCA ATC AAG GAG CGC CTC 3′-6 carboxytetramethylrhodamine (TAMRA; SEQ ID NO: 21) (Thermo Fisher Scientific), the VP35 intergenic region of BDBV probe sequence of 6FAM-5′ CGCAACCTCCACAGTCGCCT 3′-TAMRA (SEQ ID NO: 22), and the L gene of SUDV probe sequence of 6FAM-5′ CAT CCA ATC AAA GAC ATT GCG A 3′-TAMRA (SEQ ID NO: 23) were used for qRT-PCR. EBOV RNA was detected using the CFX96 detection system (BioRad Laboratories) in One-step probe qRT-PCR kits (Qiagen) with the following cycle conditions: 50° C., for 10 min, 95° C., for 10 s, and 40 cycles of 95° C., for 10 s and 57° C., for 30 s for EBOV and BDBV and 50° C., for 10 min, 95° C., for 10 s, and 40 cycles of 95° C., for 10 s and 59° C., for 30 s for SUDV. Threshold cycle (CT) values representing EBOV, BDBV and SUDV genomes were analyzed with CFX Manager Software, and data are depicted as genome equivalents (GEq); the limit of detection was 3.7 log₁₀ GEq/mL for blood and 3.7 log₁₀ GEq/g for the tissues.

NHP serum biochemistry. Serum samples collected from NHPs were tested for concentrations of albumin, amylase, alanine aminotransferase, aspartate aminotransferase, alkaline phosphatase, gamma-glutamyltransferase, glucose, blood urea nitrogen, creatinine, total protein, and C-reactive protein by using a Piccolo point-of-care analyzer and Biochemistry Panel Plus analyzer discs (Abaxis).

Sample preparation for cryogenic electron microscopy. EBOV/Makona GPAmuc was incubated overnight with a 5-fold molar excess of rEBOV-515 Fab, rEBOV-442 Fab, and rADI-16061 Fab at 4° C. The complexes were then purified by SEC using an S200I column equilibrated in 1×TBS and concentrated to 5 mg/mL using a 100-kDa concentrator (Amicon Ultra, Millipore). Immediately prior to freezing, 0.06 mM of n-Dodecyl β-D-maltoside (Anatrace) was added to 3 μL of the complex. Vitrification was performed with a Vitrobot (Thermo Fisher Scientific) equilibrated to 4° C. and 100% humidity. Cryo-EM grids were plasma cleaned for 5 s using a mixture of Ar/02 (Gatan Solarus 950 Plasma system) followed by blotting on both sides of the grid with filter paper (Whattman No. 1). See Table S7 for additional details. Note that ADI-16061 Fab was added to assist in angular sampling and orientations of the complexes in ice as the inventors described previously (Gilchuk et al., 2020b).

Cryogenic electron microscopy data collection and processing. Cryo-EM data were collected according to Table S1. Micrographs were aligned and dose-weighted using MotionCor2 (Zheng et al., 2017). CTF estimation was completed using GCTF (Zheng et al., 2017). Particle picking and initial 2D classification were initially performed using CryoSPARC 2.0 (Punjani et al., 2017) to clean up particle stacks and exclude any complexes that were degrading. Particle picks were then imported into Relion 3.1 (Zivanov et al., 2018) for 3D classification and refinement using C3 symmetry and a tight mask around the GP/rEBOV-515 Fab/rEBOV-442 Fab complex. CTF refinement was then performed by either Relion or Cryosparc to increase map quality and resolution. There was no electron density for ADI-16061 Fab.

Cryogenic electron microscopy model building and refinement. Homology models of Fab were first generated using SWISS-MODEL (Biasini et al., 2014). A model of EBOV GP (PDB: 5JQ3) was then added to generate a starting model used for refinement. The starting model was fit into the cryo-EM map using UCSF Chimera (Pettersen et al., 2004) and refined initially using Phenix real-space refinement (Liebschner et al., 2019). The refined model was then used as a starting model for relaxed refinement in Rosetta (DiMaio et al., 2015). The top five models then were evaluated for fit in EM density and adjusted manually using Coot (Emsley et al., 2010) to maximize fit. Finally, Man9 glycans were fit into glycan densities, trimmed and then a final refinement was performed in Rosetta. The final structures were evaluated using EMRinger (Barad et al., 2015) and Molprobity from Phenix. Glycans were validated using Privateer (Agirre et al., 2015) and PDBcare (Lutteke and von der Lieth, 2004). All map and model images were generated in UCSF Chimera (Pettersen et al., 2004). Antibody contacts were analyzed using LigPlot (Laskowski and Swindells, 2011), Arpeggio (Jubb et al., 2017) and UCSF Chimera (Pettersen et al., 2004).

Quantification and Statistical Analysis. The descriptive statistics mean±SEM or mean±SD were determined for continuous variables as noted. Survival curves were estimated using the Kaplan-Meier method and overall difference in survival between the groups in mouse studies was estimated using two-sided log rank test (Mantel-Cox) with subjects right censored, if they survived until the end of the study. In NHP studies, survival curves were estimated using the Kaplan-Meier method, and the proportion surviving at day 28 after virus inoculation was compared using a 2-sided exact unconditional test of homogeneity. Curves for antibody binding were fitted after log transformation of antibody concentrations using a four-parameter log-logistic (4PL) analysis. In neutralization assays and GP cleavage inhibition assays, IC₅₀ values were calculated after log transformation of antibody concentrations using a four-parameter log-logistic (4PL) analysis. Synergy distribution maps were generated from the dose-response binding matrix using an open-source software SynergyFinder 2.0: visual analytics of multi-drug combination synergies (synergyfinder.fimm.fi), and data was analyzed using ZIP synergy scoring model (lanevski et al., 2020). Technical and biological replicates are indicated in the figure legends. Statistical analyses were performed using Prism v8.4.3 (GraphPad).

Data and Code Availability. The EBOV GP ΔMuc ΔTM (Makona)-rEBOV-515-rEBOV-442 Fab cryo-EM structure has been deposited in the PDB with accession code 7M8L. The accession number for cryo-EM reconstructions reported in this paper have been deposited to the Electron Microscopy Data Bank under accession EMDB code EMD-23719 (see Key Resources Table for details). All data needed to evaluate the conclusions in the paper are present in the paper or the Supplemental Information; source data for each of the display items is provided in Key Resources Table.

### Example 2-Results

Activities of pan-ebolavirus candidate cocktail containing recombinant antibodies rEBOV-442 and rEBOV-515. Antibody variable gene sequences for mAbs EBOV-515 and EBOV-442 were determined, and synthetic DNAs encoding the mAbs were used to produce recombinant IgG1 in transiently transfected Chinese hamster ovary (CHO) cells. Recombinant (r) mAbs designated as rEBOV-515 and rEBOV-442 potently neutralized EBOV, BDBV, and SUDV (FIG. 1A) with a note that rEBOV-442 was more active against Boniface variant of SUDV (see below). To assess the Fc-mediated effector function of these antibodies to mediate killing of antigen-expressing cells, the inventors used a stably-transfected EBOV GP-expressing SNAP-tagged 293F cell line as a target, with heterologous human PBMCs as source of effector cells and a previously described assay (Domi et al., 2018). The glycan-cap-specific antibody rEBOV-442 IgG1 exhibited a high level of cytotoxic activity, and the GP-base-specific antibody rEBOV-515 exhibited a moderate level of cytotoxic activity, relative to the control Fc-function-disabled IgG1-LALA-PG variant of rEBOV-515 (designated as rEBOV-515 LALA-PG) or an irrelevant antibody rDENV 2D22 (IgG1 isotype) specific to the dengue virus envelope (E) protein (Fibriansah et al., 2015) (FIG. 1B). Neutralizing activity of the wild-type IgG1 and IgG1-LALA-PG variants of rEBOV-515 was equivalent (FIG. S1A). These results taken together revealed two complementary activities exhibited by this broad antibody cocktail, specifically the Fc-mediated effector functions mediated by rEBOV-442 and the potent neutralizing activities mediated by rEBOV-442 and rEBOV-515 (FIG. 1C).

One benefit of using a cocktail of two or more neutralizing antibodies that bind to non-overlapping regions of the viral antigen is to reduce the risk of viral escape from neutralization that is inherent in monotherapy approaches (Baum et al., 2020; Misasi and Sullivan, 2021; Yewdell et al., 1979). To demonstrate this feature for this cocktail directly, the inventors next used a recombinant infectious vesicular stomatitis virus (VSV) expressing EBOV GP in place of the endogenous VSV glycoprotein (VSV/EBOV GP) to select for GP mutations that escape antibody neutralization. The inventors assessed rEBOV-442, rEBOV-515, or 1:1 mixture of both antibodies (half maximal inhibitory concentration (IC₅₀)<1.5 μg/mL against EBOV for individual mAbs and the cocktail) and used a high-throughput quantitative real-time cellular analysis assay (RTCA) to select viral variants that can escape neutralization (Gilchuk et al., 2020b; Greaney et al., 2021). They selected for escape at a single saturating antibody concentration of 20 μg/mL using ˜20,000 plaque forming units of VSV/EBOV GP per well (˜1 multiplicity of infection [MOI]) and performing 60 replicates for each sample (FIG. 1D). For rEBOV-442, this process selected viral variants that they confirmed escaped neutralization at 20 μg/mL of rEBOV-442 but were neutralized by rEBOV-515 or a mixture of rEBOV-442 and rEBOV-515 (FIG. 1D, FIG. S2B). The inventors sequenced the viral gene encoding the GP of rEBOV-442-selected escape viruses and identified seven distinct point mutations, including the previously identified escape mutation L273P (Gilchuk et al., 2018a) that appeared in all sequenced escape viruses in this study (FIG. 1D). The escape mutation L273P likely pre-existed in the virus stock at a low frequency explaining the observed rate of escape from rEBOV-442 neutralization. In all cases, escape viruses carried GP mutations in the binding site of rEBOV-442 (FIG. S1C). For rEBOV-515 or the cocktail, escape variants were not detected in any of the 60 replicate experiments (FIG. 1D). Together these results demonstrated a high resistance to viral escape for the cocktail of the two broad antibodies rEBOV-442 and rEBOV-515, which acts by both neutralizing and Fc-mediated activities.

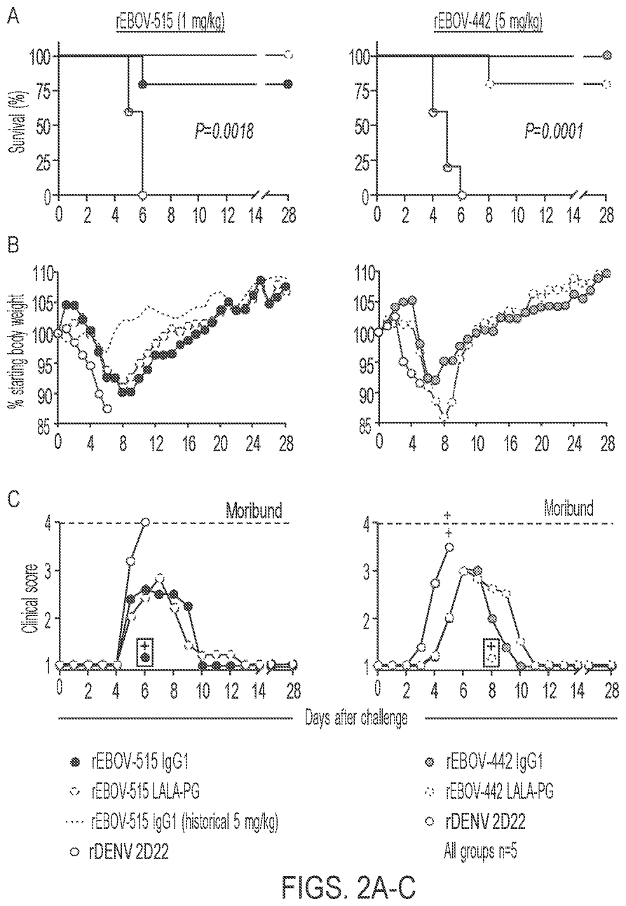

Differential requirements for the Fc regions of antibodies rEBOV-442 and rEBOV-515 for therapeutic protection in mice. To define the contribution of Fc-mediated effector functions to protection in vivo, each mAb of the candidate therapeutic cocktail was assessed as an IgG1 protein (the original subclass of antibody secreted by the hybridoma cell line and a functionally competent form of mAb) and as IgG1 LALA-PG protein (which is disabled for Fc function and fully silent in mice). The inventors challenged groups of mice with mouse-adapted EBOV (EBOV-MA) on day 0 and administered antibodies 1 day later. Previously, the showed that for low-dose treatment (1 mg/kg), the Fc-disabled LALA variant of the GP base-specific mAb EBOV-520 offered a higher level of therapeutic protection in mice against EBOV (60% survival) when compared to that mediated by the wild-type (wt) IgG1 EBOV-520 (0% survival; (Kuzmina et al., 2018)). In a new study conducted here, the rEBOV-515 LALA-PG antibody variant offered complete protection (100% survival) against EBOV challenge in mice at a 1 mg/kg therapeutic dose, while the wt IgG1 variant showed partial protection (80% survival) (FIGS. 2A-C). This finding demonstrated a higher in vivo potency of the broad GP base-specific mAb rEBOV-515 when compared to the previously reported effect of the broad GP base-specific mAb rEBOV-520, and suggested that rEBOV-515 LALA-PG is a preferable Fc variant for the cocktail of two Fc variants tested. Similarly, the inventors compared the in vivo potency of wt IgG1 rEBOV-442 and rEBOV-442 LALA-PG, which were administered at a 5 mg/kg dose one day after EBOV challenge (FIGS. 2A-C). Cytotoxicity assay results (FIG. 1B) suggested that the glycan cap antibody rEBOV-442, in addition to possessing neutralizing activity, also exhibited substantial Fc effector function-mediated activity. In agreement with this finding, the results of the in vivo study showed a transient (2-4 days post-infection) improvement in weight (i.e., reduced loss) with wt rEBOV-442 IgG1, which was associated with improved protection (100% survival in the wt antibody-treated group when compared to 80% in the rEBOV-442 LALA-PG-treated group) (FIGS. 2A-C). Together, these data revealed differing requirements for the Fc regions of rEBOV-442 and rEBOV-515, demonstrated a high in vivo potency of the antibodies tested, and justified the inclusion of rEBOV-515 as a LALA-PG variant and rEBOV-442 as a wt IgG1 variant in a new broad and protective ebolavirus two-antibody cocktail.

Synergistic activity of antibodies rEBOV-442 and rEBOV-515 in the cocktail. Antibodies in a cocktail directed to a common viral protein may recognize antigen in a synergistic, additive, or antagonistic manner. The inventors first characterized the effect mediated by the mixture of two antibodies on ebolavirus GP binding. Serially diluted Alexa-Fluor-647-labeled antibody rEBOV-442 IgG1 or rEBOV-515 LALA-PG was titrated into serially-diluted unlabeled partner antibody to generate two pairwise combinatorial matrices of two antibodies in the mixture (FIGS. S2A-B). Binding of the Alexa-Fluor-647-labeled antibody from each combination was assessed by flow cytometric analysis using a Jurkat cell line that was stably transduced to display EBOV GP on the surface as described before (Gilchuk et al., 2020b). The values were calculated as the percent of the relative fluorescence signal caused by the highest concentration of respective fluorescently-labeled antibody alone (FIGS. S2A-B), and then compared with the expected responses calculated using the ZIP synergy-scoring model (Ianevski et al., 2020). The synergy score can be interpreted as the average excess response (percent change) for an antibody combination (Ianevski et al., 2020). The comparison revealed that the combination of antibodies rEBOV-442 and rEBOV-515 was synergistic, and each antibody reciprocally enhanced binding of the partner antibody in the mixture (FIGS. S2B-C). This finding contrasted with the unidirectional GP binding synergy observed in the cocktail of the other broad antibodies rEBOV-520 and rEBOV-548 (Gilchuk et al., 2020). Binding enhancement increased steadily with increasing of antibody concentration, indicating more contribution from synergy at higher concentrations of individual Abs in the cocktail (FIGS. S2B-C).

The inventors next characterized the effect by the mixture of two antibodies on ebolavirus neutralization. Using a recently developed real-time cell analysis (RTCA) cellular impedance assay that quantifies virus-induced cytopathic effects and infectious chimeric VSV/ebolavirus GP viruses (Gilchuk et al., 2020a; Gilchuk et al., 2020b), the inventors showed efficient and dose-dependent neutralization of VSV/EBOV-GP,/BDBV-GP, or/SUDV-GP viruses by the 1:1 antibody mixture (FIG. 3A). Then, they titrated serially-diluted rEBOV-442 into serially-diluted rEBOV-515 to generate a pairwise combinatorial matrix of two antibodies in the mixture and assessed each combination for neutralization of VSV/EBOV-GP,/BDBV-GP, or/SUDV-GP viruses (FIG. 3B). Data were analyzed using the ZIP synergy scoring model. This analysis identified areas in the neutralization matrix (showing mAb ratios) with the most profound combination effect and suggested broad synergistic activity in the cocktail for the mixture of rEBOV-442 and rEBOV-515 (FIG. 3C).

Protective pan-ebolavirus combination therapy of nonhuman primates by antibodies rEBOV-442 and rEBOV-515. Next, the inventors tested the efficacy of the cocktail of rEBOV-515 LALA-PG+rEBOV-442 IgG1 in nonhuman primate (NHP) challenge models for each of the three ebolaviruses, EBOV (Kikwit variant), BDBV (Uganda variant), and SUDV (Gulu variant). They used rhesus macaque EBOV and SUDV lethal challenge models and a cynomolgus monkey BDBV lethal challenge model, which recapitulate many key features of EVD in humans (Bennett et al., 2017; Geisbert et al., 2015). Animals were assigned to three treatment groups of five animals per group. After intramuscular challenge with a lethal target dose of 1,000 plaque-forming units (PFU) of EBOV, BDBV, or SUDV, all NHPs of the treatment group received intravenously two 30 mg/kg doses of the cocktail (a 2:1 mixture of rEBOV-515 LALA-PG and rEBOV-442 IgG1) spaced 3 days apart (given days 3 and 6 after EBOV, days 6 and 9 after BDBV, or days 4 and 7 after SUDV inoculation). The invent tors chose a 2:1 antibody ratio in the cocktail based on the high level of synergy identified for this antibody ratio from the in vitro synergy distribution maps (FIGS. 3A-C;

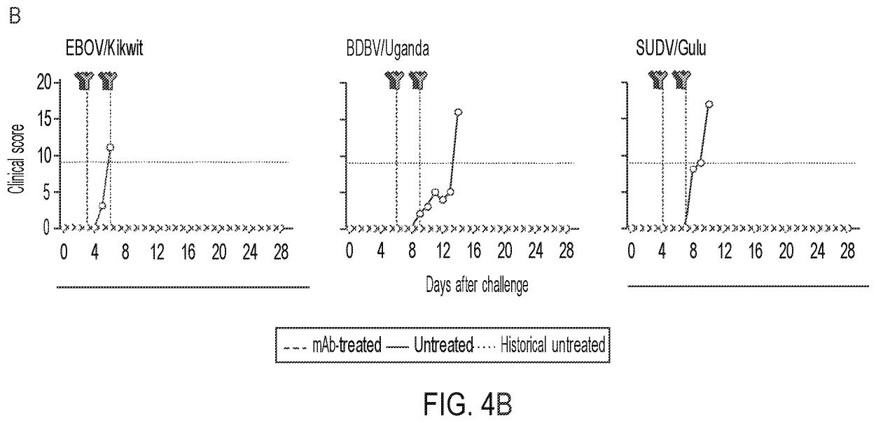

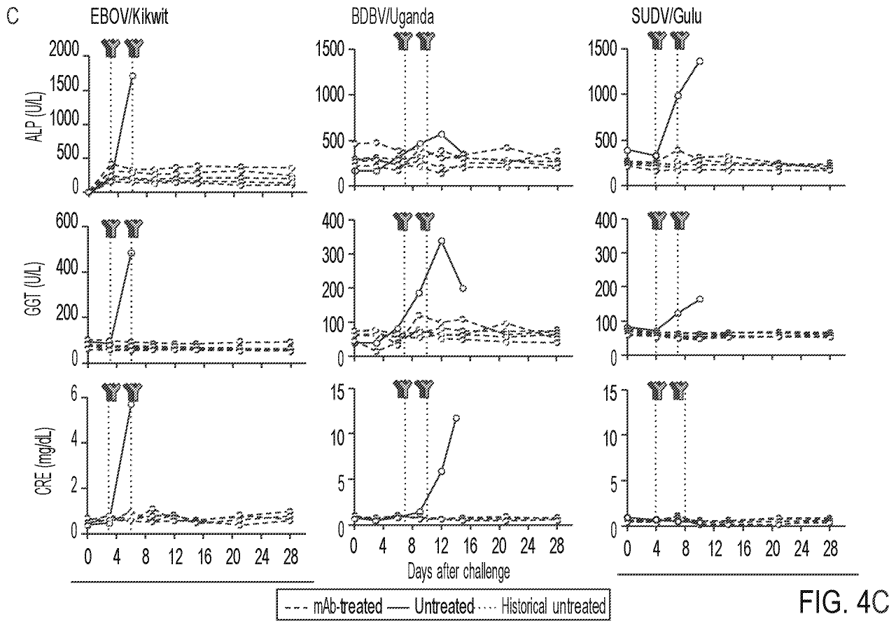

FIG. S2A-C) and due to higher neutralizing potency of rEBOV-515 against SUDV when compared to the potency of rEBOV-442 (FIG. 1A). For each challenge cohort, an additional animal was studied as a contemporaneous control and was left untreated. The untreated control animals developed a high clinical score and succumbed to disease on day 6, 14, or 10 after viral challenge with EBOV, BDBV, or SUDV, respectively. The two-dose therapeutic cocktail treatment provided complete pan-ebolavirus protection of NHPs from mortality and clinical signs of EVD (FIGS. 4A-B). The inventors next assessed changes in blood chemistries and blood cell composition that are typically associated with EVD to further characterize the efficacy of the mAb cocktail treatment (Tables S1-6). The liver enzymes alkaline phosphatase (ALP) and gamma glutamyl transferase (GGT), which are indicators of EVD (Qiu et al., 2014), were elevated in untreated NHPs at the peak of the disease (FIGS. 4B-C). Treated animals did not show signs of acute liver injury after the treatment, displaying low amounts of ALP, GGT, creatinine (CRE), and the other blood chemistries when compared with those of untreated NHP (FIG. 4C; Tables S4-6).

At the time of first treatment with the cocktail (day 3 after EBOV, day 6 after BDBV, or day 7 after SUDV inoculation), 14 NHPs from the treatment-designated group (all except one animal in EBOV-challenged group) and the control untreated NHPs were viremic, with virus titers that ranged from 5.1 to 10.6 log₁₀ genome equivalents (GEQs) per mL of plasma, as measured by quantitative reverse-transcription PCR (qRT-PCR) (FIG. 5A). A plaque assay that detects infectious virus revealed viremia in all 18 animals, with viral titers ranging from 0.7 to 7.4 log₁₀ plaque forming units (PFU) per mL of plasma at the time of first treatment with the cocktail (FIG. 5B). This finding confirmed active ebolavirus infection in all NHPs before treatment. At the time of the second antibody cocktail treatment (day 6 after EBOV, days 9 after BDBV, or day 7 after SUDV inoculation), each untreated control NHP from a respective EBOV, BDBV, or SUDV challenge cohort developed high viremia >8 log₁₀ GEQ/mL and >5 log₁₀ PFU/mL (FIGS. 5A-B). Concordant with full protection from disease and death, none of the 15 treated NHPs had detectable infectious virus in the plasma at that time (limit of detection [LOD]=5 PFU/mL), and viral genomes were not detected in 12 of those NHPs 3.7 log₁₀ GEQ/mL at that time (FIGS. 4A-C, FIGS. 5A-B). The three remaining NHPs that were from the BDBV inoculation cohort and that showed slightly delayed clearance of viral genomes in plasma no longer had detectable virus by day 15 after viral challenge. Analysis of various tissues harvested at study endpoint from treated animals (day 28 after inoculation with EBOV and SUDV or day 35 after inoculation with BDBV) and untreated animals (day 6, 14, or 10 after inoculation with EBOV, BDBV, or SUDV, respectively) confirmed virologic protection mediated by the cocktail treatment (FIG. S3).

Together these results showed a high therapeutic efficacy of the cocktail of rEBOV-515 LALA-PG+rEBOV-442 IgG1 to treat and revert disease caused by primary ebolaviruses that are responsible for outbreaks in humans-EBOV, BDBV, and SUDV.

Structural basis for the efficacy and broad ebolavirus neutralization by the cocktail. The inventors previously reported the molecular determinants of the GP binding for several glycan cap-specific antibodies, including the broad antibody rEBOV-442 (Murin et al., 2021), and defined the structural basis of synergy for the pair of broad antibodies rEBOV-520 and rEBOV-548 (Gilchuk et al., 2020b). In this new study, the inventors focused on studies of the determinants of rEBOV-515 binding to elucidate the structural basis for the neutralization breadth and efficacy mediated by pan-ebolavirus cocktail of rEBOV-515+rEBOV-442. They generated a complex of both rEBOV-515 and rEBOV-442 Fab with mucin-deleted EBOV GP from the Makona variant (EBOV GPΔMuc/Mak) and solved structures by cryogenic electron microscopy (cryo-EM) (Table S7; FIG. 6A and FIGS. S4A-C).

The interface between GP and rEBOV-515 in this structure was resolved to ˜3 to 3.5 Å resolution (FIGS. S4A-C), allowing for confident modeling of most of the side-chain residues. rEBOV-515 binds to the base of the internal fusion loop (IFL), directly below the glycan cap in GP1 (FIG. 6A). There is significant overlap of the rEBOV-515 epitope with that of rEBOV-520, but rEBOV-515 has more extensive contacts in GP2 and less in GP1 (FIG. 6B). Most EBOV-515 contacts are mediated by the heavy chain (HC), with all three complementarity determining regions (CDRs) contributing to binding the base of the IFL as well as a portion of GP1 near the 310 pocket (FIG. 6C; FIGS. S5A-B, FIG. S6). The CDRH3 contributes the most extensive contacts, including those that displace the descending β17-β18 loop of the glycan cap, making contacts near the 310 pocket, as well as a strong cation-pi bond with R136_{GP1} (FIG. 6C). The rEBOV-515 light chain (LC) also makes contacts with all three CDRs exclusively within GP2 (FIG. 6D; FIG. S5A). Together, this analysis showed that rEBOV-515 forms a strong interaction of complementary hydrophobic (FIG. S5B) and electrostatic (FIG. S5C) surfaces for binding to this epitope within GP.

Neutralizing potency may depend on the ability of antibodies to remain bound to viral GP within the acidic environment of late endosomes, which is where pH-dependent cleavage occurs to expose the Niemann-Pick C1 (NPC1) receptor binding site (Carette et al., 2011; Chandran et al., 2005). Given that rEBOV-515 strongly interacts with the EBOV GP, the inventors assessed binding of this antibody at varying pH to recombinant EBOV, BDBV or SUDV GPs by ELISA. The inventors used the broadly reactive base-specific antibody rEBOV-520 for comparison. rEBOV-515 and rEBOV-520 bound equivalently to the GP of each of the three ebolaviruses at neutral pH 7.4, and rEBOV-515 remained bound at acidic pH of 5.5 or 4.5, while rEBOV-520 lost binding activity (FIG. S7A).

The rEBOV-515 binding site partially overlaps with those of other reported broad antibodies that bind to the 310 pocket, including rEBOV-520 (Gilchuk et al., 2020b) and ADI-15946 (West et al., 2019) (FIGS. 6B and 6E). Each of these antibodies relies upon E106 and R136 in GP1 and several residues at the base of the IFL, using long CDRH3 loops that mimic and replace the β17-β18 loop of the GP1 in apo-GP structure (FIG. 6E). However, characterization of the rEBOV-515 binding site revealed several unique features. rEBOV-515 uses a single CDR loop to simultaneously mimic and displace the 17-B18 loop and to bind to R136 in GP1. This feature allows rEBOV-515 to access a region that is occupied by a glycerol cryoprotectant molecule in the unliganded crystal structure of GP (Protein Data Base [PDB] 5JQ3; 5JQB) that the inventors defined as “glycerol pocket” (FIG. 6E). In addition, this mechanism facilitates greater interaction of EBOV-515 with the cleavage loop compared to that caused by rEBOV-520, which may explain the high GP cleavage-inhibiting activity of EBOV-515 (FIG. S7B). Further, a distinct pose of rEBOV-515 binding into the 310 pocket avoids clashes with the α2 helix of the glycan cap (FIG. 6F), unlike the binding of rEBOV-520 to the GP, which requires the α2 helix shift (Gilchuk et al., 2020b). This finding suggested that the binding site of rEBOV-515 on the intact GP molecule is more accessible than the rEBOV-520 binding site, which could explain the higher neutralizing potency of rEBOV-515 when compared to the potency of rEBOV-520 against the virus carrying the intact GP. Of note, the inventors have shown previously that GP cleavage, which removes the glycan cap along with the β17-β18 loop, resulted in enhanced binding to cell-surface-displayed cleaved GP and increased neutralizing potency for both rEBOV-515 and rEBOV-520 (Gilchuk et al., 2018a; Gilchuk et al., 2020b).

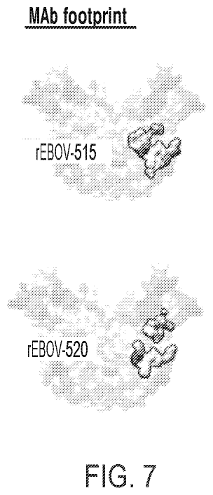

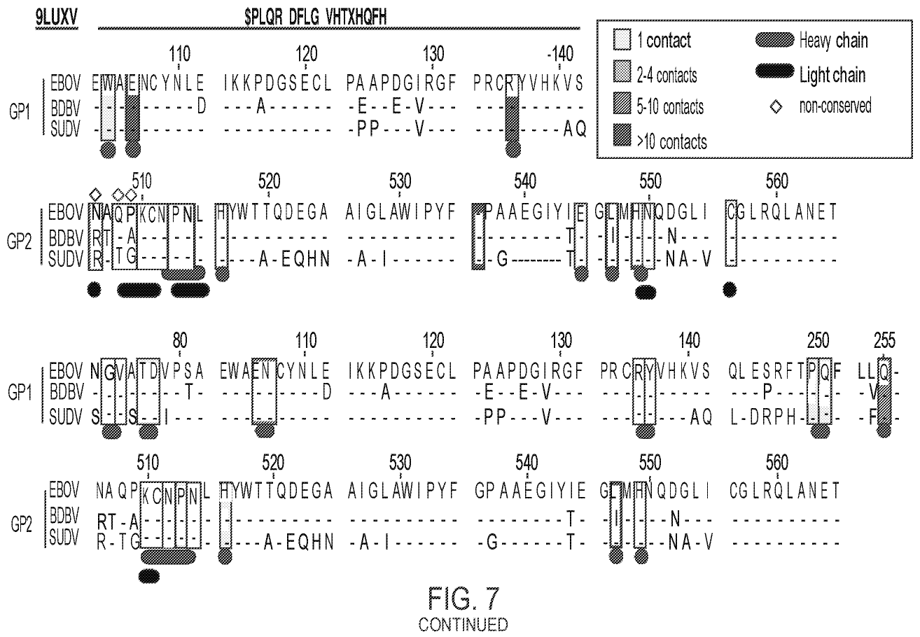

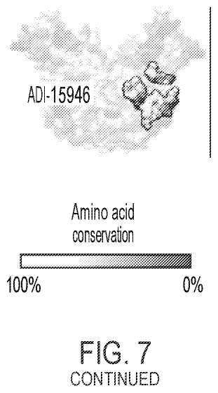

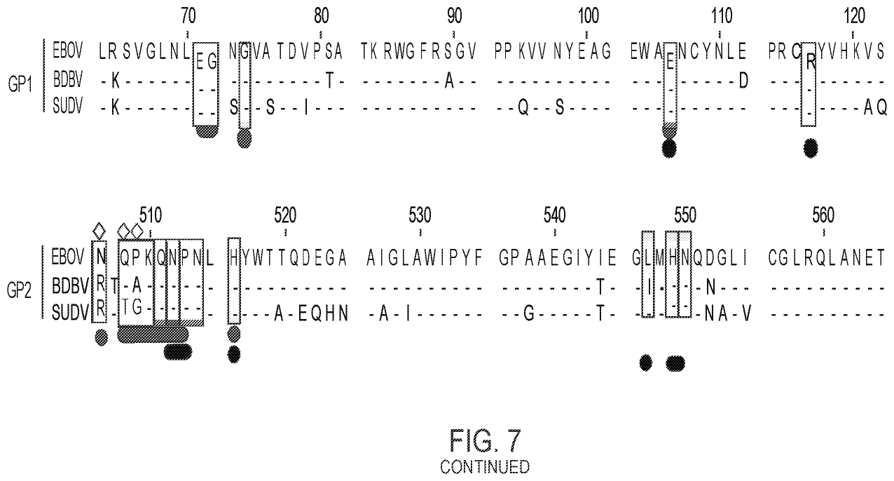

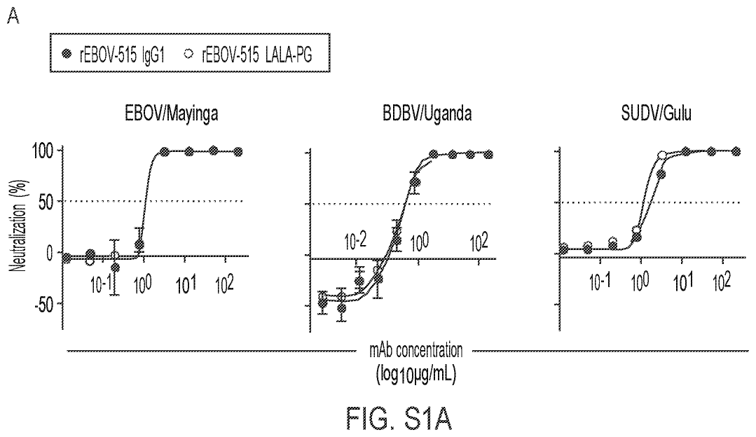

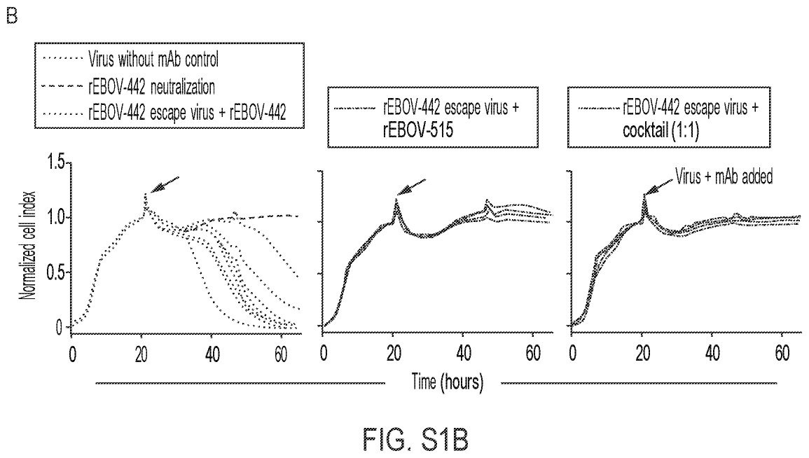

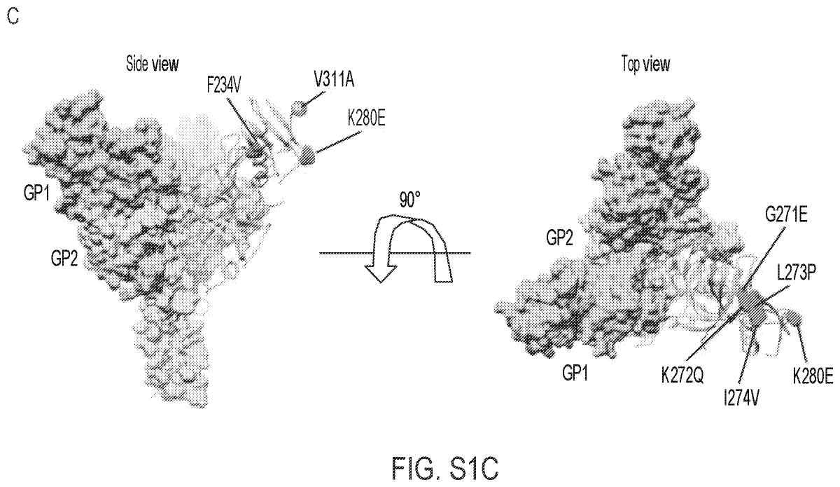

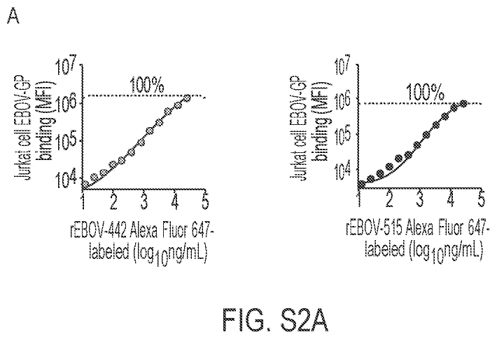

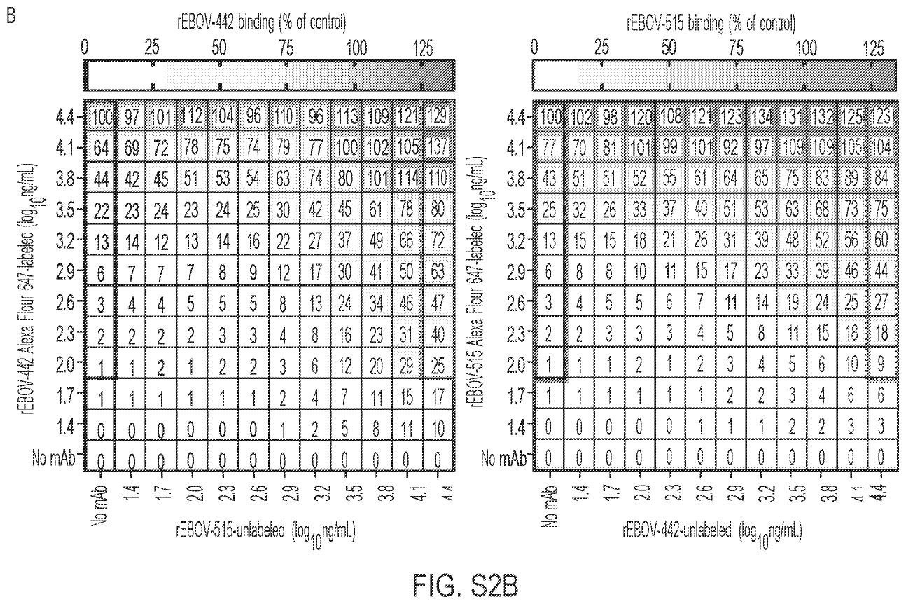

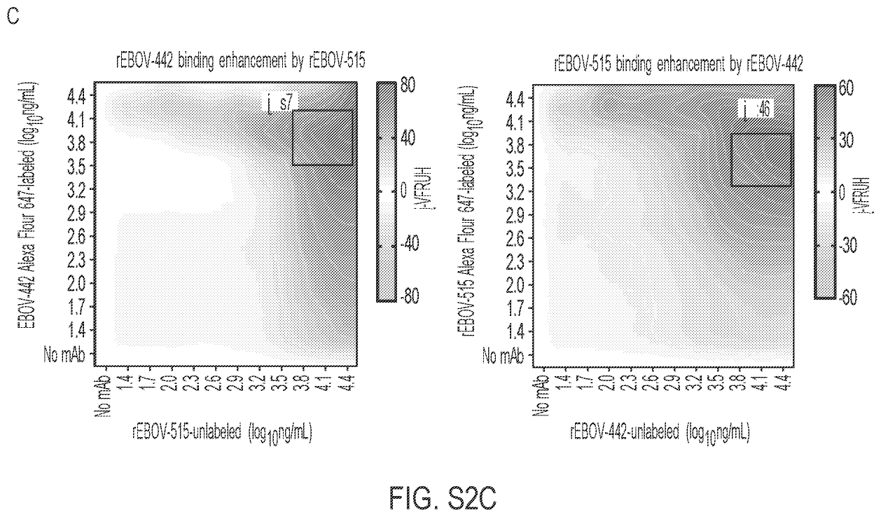

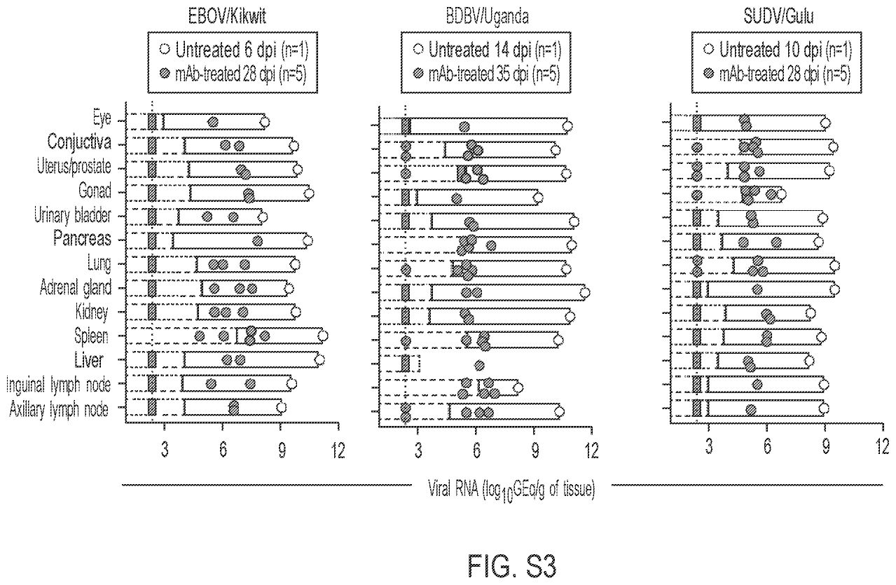

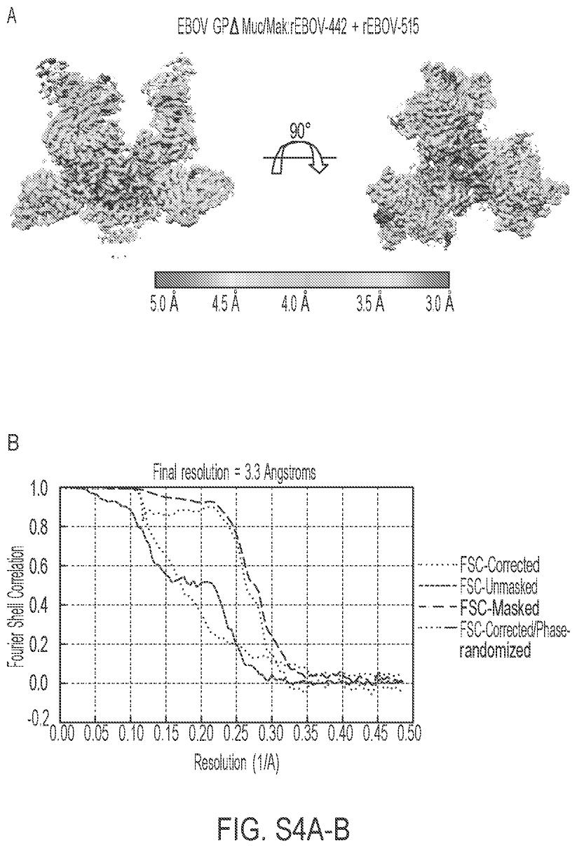

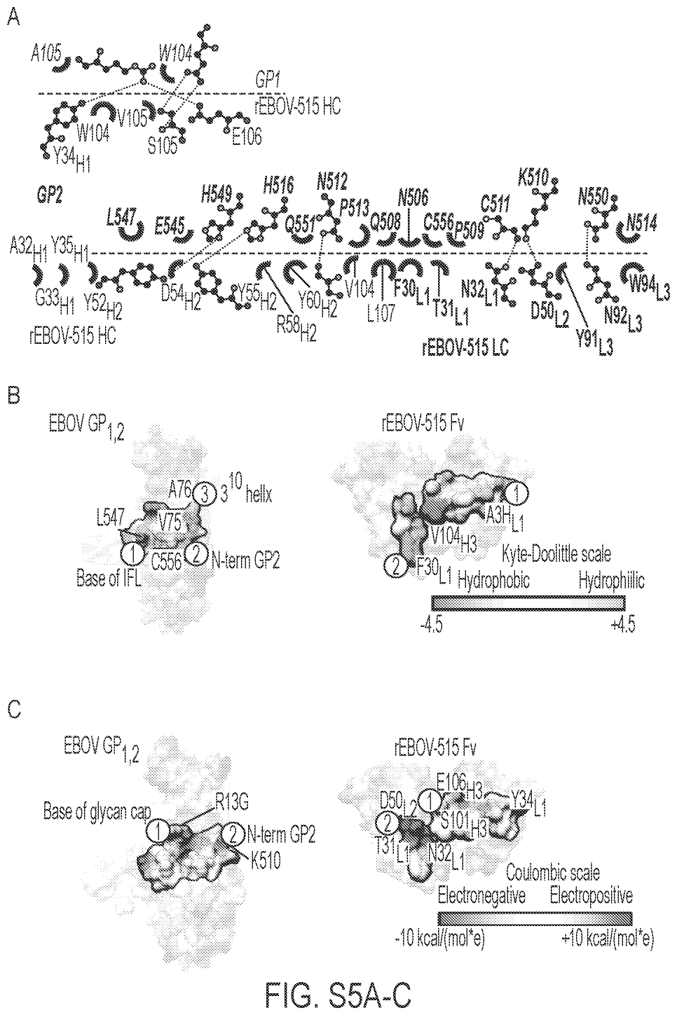

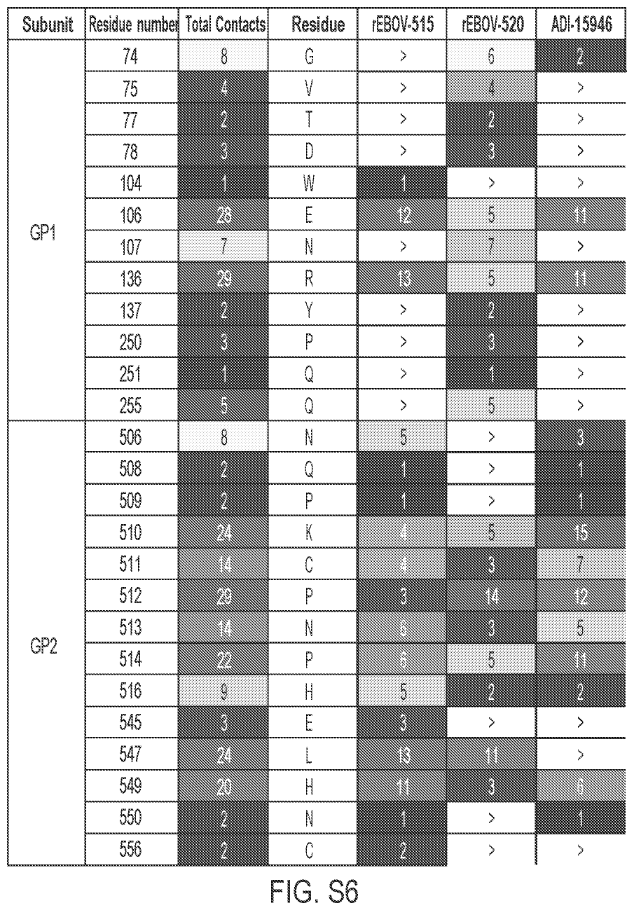

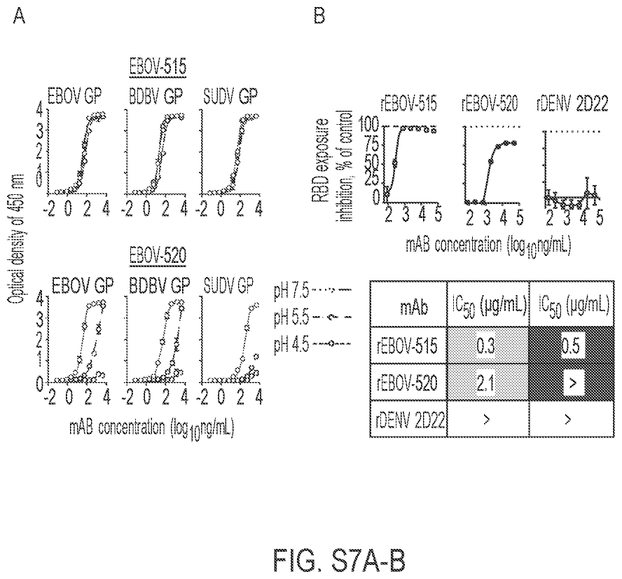

There are notable differences in degree of pan-ebolavirus neutralization and protection by each of three reported broadly reactive human antibodies despite recognition of partially overlapped epitopes they recognize on the GP base (FIG. 6B). ADI-15946 did not fully neutralize most antigenically distinct SUDV (Wec et al., 2017; West et al., 2019), rEBOV-520 neutralized SUDV with modest potency but showed weak protection against SUDV in mice, and rEBOV-515 potently neutralized each of three viruses and protected against SUDV in mice (Gilchuk et al., 2018a; Gilchuk et al., 2020b). A structural analysis allowed the inventors to compare the epitope footprint and contacts for each of these antibodies in relation to sequence conservation between the three major ebolavirus (FIG. 7; FIG. S6), providing insight that may help explain differences in neutralization breadth. ADI-15946 makes fewer contacts with GP1 but more contacts to the N-terminus of GP2, a region that is less conserved that may explain weak SUDV neutralization. Conversely, rEBOV-520 makes more extensive contacts with GP1, as this region is generally less conserved. rEBOV-515 relies on minimal contacts with GP1, except in regions that are completely conserved, including R136 and E106. Taken together with a more conserved footprint, these observations provide a molecular basis for the superior breadth of neutralization and protection exhibited by rEBOV-515 over similar antibodies.

### Example 3-Discussion

Here the inventors report comprehensive studies of a new pan-ebolavirus antibody combination treatment with a well-defined and complex molecular basis of broad neutralization and potency. The cocktail exhibited high therapeutic efficacy against all three medically important ebolaviruses in nonhuman primates. The discovery and recent approval of human antibody-based therapeutics represents a landmark achievement in the development of EVD medical countermeasures. The clinical trials in the DRC outbreak, however, highlighted substantial gaps remained in improving the treatment of acute EVD (Iversen et al., 2020). The greatest benefit of antibody treatment in patients was mainly in those receiving early therapy, and only moderate benefit was observed in severely ill patients (Levine, 2019). Another remaining challenge for implementation of the current regimens is the logistical complexity of intravenous drug administration in the field, which may limit widespread application of antibody therapy in future outbreak scenarios.

Given the difference in efficacy mediated by different antibody drugs that was observed for advanced EVD treatments, antibody potency likely is a key contributing determinant of treatment efficacy. Inmazeb and Ebanga demonstrated higher efficacy when compared to that of the antibody cocktail ZMapp™ in clinical trials (Levine, 2019). Inmazeb is a three-antibody cocktail based on REGN-EB3 antibody sequences (Pascal et al., 2018), Ebanga is a monotherapy based on the antibody mAb114 (Corti et al., 2016), and ZMapp™ is a cocktail of three murine-human chimeric mAbs (Qiu et al., 2014). A comparison with the historical NHPs studies with REGN-EB3 (3×50 mg/kg dose at d5, 8, and 11 after exposure) (Pascal et al., 2018), mAb114 (3×50 mg/kg dose at d1, 2, and 3 after exposure) (Corti et al., 2016), and ZMapp™ (3×50 mg/kg dose at d3, 6, and 9 after exposure) (Qiu et al., 2014) suggested equivalent or likely higher potency of the inventors' EBOV-442 IgG1+EBOV-515 LALA-PG cocktail treatment (2×30 mg/kg at d3 and 6 after exposure) against homologous EBOV. More studies are needed to compare available treatments and to determine if increasing antibody therapeutic potency would benefit clinical outcomes in treatment of severely ill patients and/or allow for rapid and more practical treatment by an alternative (intramuscular or subcutaneous) route of antibody delivery.

Anticipation of future EVD outbreaks requires consideration of therapeutic breadth. As an added feature, the two-antibody cocktail the inventors describe here offered an increased therapeutic breadth that extended to protection against BDBV and SUDV in NHP models. One other investigational human mAb cocktail that demonstrated broad efficacy in NHPs, MBP134AF, has been described (Bornholdt et al., 2019). MBP134AF is comprised of antibody ADI-15878 and a derivative of antibody ADI-15946 (defined as ADI-23774), which was engineered to improve its specificity against SUDV GP (Wec et al., 2019). A comparison of the inventors' results to the reported activities of ADI-15946 or ADI-23774 (Wec et al., 2017) indicates a high potency for the homologous base-specific antibody rEBOV-515 that the inventors describe. In agreement with this functional assessment, the inventors' structural data showed that rEBOV-515 strongly interacts with conserved residues in the GP, with a unique footprint among base-specific broadly reactive human mAbs rEBOV-515, rEBOV-520, and ADI-15946, suggesting a structural basis for its remarkable breadth and potency.

Multifunctionality is another desirable feature in antibody cocktails in addition to neutralizing potency and breadth (Saphire et al., 2018). Reports of human EVD cases revealed high plasma viral RNA titers at the time of patient admission into antibody treatment studies (Brown et al., 2018; Mbala-Kingebeni et al., 2019). Similarly, the inventors observed high serum viral titers in each cohort of challenged NHPs before antibody treatment (FIGS. 5A-B), indicating that at the time of treatment many cells in multiple organs are already infected. This finding highlights the importance to retain Fc-mediated effector function activity in antibody cocktails in order to preserve the ability to eliminate infected cells. In the cocktail of EBOV-442 IgG1+EBOV-515 LALA-PG, the stronger and broader neutralizing antibody EBOV-515 LALA-PG may act directly to neutralize circulating virus, and this activity is enhanced synergistically in the presence of rEBOV-442 IgG1. Antibody rEBOV-442 IgG1, in addition to direct virus neutralization, may act through Fc-mediated functions by binding to the non-overlapping antigenic site on the GP on infected cells, and this activity could be enhanced reciprocally in the presence of EBOV-515 LALA-PG. Such a combination of activities may facilitate viral clearance and potentially decrease the likelihood of viral mutations that facilitate escape from the cocktail treatment. The relative contribution of these activities to the therapeutic efficacy exhibited by this cocktail needs further investigation.

During acute EVD, circulating infectious virus sometimes seeds immune-privileged tissues, including the brain, eyes, and testes, and persist after clearance from the blood and recovery (Diallo et al., 2016; Subtil et al., 2017; Varkey et al., 2015). Until recently, it was generally assumed that Ebola epidemics start upon zoonotic transmission. On Feb. 14, 2021, a new EVD outbreak was declared in Guinea, and viral genome sequencing reports suggested that the outbreak was caused by the Makona variant of EBOV that caused the 2014 EVD epidemics (virological.org, 2021). The index case of the 2021 Guinea cluster likely was infected from a persistent source, such as via sexual transmission from an EVD survivor (virological.org. 2021), raising concerns about possible person-to-person transmission and reignition of outbreaks. The existing therapies and those that are currently in clinical development should be evaluated for their efficacy in clearing infectious virus from immune-privileged sites. Further, NHP studies suggested a genetic drift upon selection pressure with sub-optimal antibody treatment that could be a potential cause for failure during EVD treatment (Kugelman et al., 2015). Potent antibody cocktails like EBOV-442 IgG1+EBOV-515 LALA-PG may help thwart antigenic drift by targeting non-overlapping vulnerable sites on GP and exhibiting complementary mechanisms of action.

In summary, these studies highlight the power of implementing a rational mAb cocktail development program using structure-function-guided principles (e.g., knowledge of binding sites, neutralization breadth, resistance to escape, multifunctionality, synergy, etc.). The inventors identified a pan-ebolavirus biologic comprising a two-antibody cocktail that exhibits high efficacy for treatment of all three medically important ebolaviruses with only two doses of mAb. These findings set the stage for clinical evaluation of pan-ebolavirus combination therapy with the two human antibodies rEBOV-442 IgG1 and rEBOV-515 LALA-PG.

TABLE S1
Hematology measurements from rEBOV-515 + rEBOV-442 cocktail-treated
or -untreated individual NHP that were challenged with EBOV. Related to FIG. 4

| | | WBC | RBC | | | | | | | | | | | |
|---|---|---|---|---|---|---|---|---|---|---|---|---|---|---|
| | | per | per | | | Platelets | Lymph | | Mono. | | | | | |
| Animal | Day after | mm³ | mm³ | HgB | Hct | per mm³ | per mm³ | Lymph. | per mm³ | Neutr. | Eosin. | Baso. | MCH | MPV |
| ID | challenge | (×10³) | (×10³) | (g/dL) | (%) | (×10³ | (×10³) | (%) | (×10³) | (%) | (%) | (%) | (pg/cell) | (fL/cell) |
| M1 | 0 | 8 | 5 | 11 | 37 | 326 | 3 | 30 | 63 | 6 | 1 | 0 | 20 | 9 |
| M2 | 0 | 4 | 5 | 10 | 35 | 210 | 3 | 67 | 15 | 18 | 1 | 0 | 20 | 11 |
| M3 | 0 | 7 | 5 | 10 | 35 | 225 | 5 | 77 | 8 | 13 | 2 | 1 | 20 | 10 |
| M4 | 0 | 6 | 6 | 13 | 46 | 374 | 3 | 55 | 23 | 18 | 2 | 1 | 21 | 10 |
| M5 | 0 | 3 | 6 | 12 | 44 | 321 | 2 | 70 | 24 | 4 | 2 | 1 | 20 | 9 |
| C1 | 0 | 7 | 5 | 12 | 42 | 312 | 3 | 46 | 1 | 51 | 2 | 1 | 23 | 11 |
| M1 | 3 | 7 | 5 | 11 | 33 | 250 | 4 | 66 | 29 | 4 | 1 | 0 | 23 | 9 |
| M2 | 3 | 6 | 5 | 11 | 35 | 182 | 4 | 67 | 20 | 11 | 1 | 0 | 21 | 10 |
| M3 | 3 | 9 | 6 | 12 | 42 | 122 | 6 | 66 | 25 | 8 | 1 | 0 | 19 | 11 |
| M4 | 3 | 10 | 5 | 12 | 40 | 345 | 2 | 20 | 1 | 77 | 2 | 1 | 22 | 9 |
| M5 | 3 | 6 | 4 | 11 | 31 | 248 | 1 | 24 | 40 | 34 | 2 | 1 | 26 | 8 |
| C1 | 3 | 15 | 5 | 12 | 45 | 258 | 3 | 18 | 1 | 79 | 2 | 1 | 22 | 11 |
| M1 | 6 | 5 | 5 | 11 | 37 | 256 | 4 | 82 | 14 | 3 | 1 | 0 | 20 | 8 |
| M2 | 6 | 5 | 5 | 10 | 37 | 189 | 3 | 65 | 15 | 18 | 2 | 0 | 20 | 11 |
| M3 | 6 | 6 | 5 | 10 | 35 | 234 | 5 | 84 | 13 | 1 | 2 | 1 | 20 | 9 |
| M4 | 6 | 6 | 6 | 12 | 41 | 333 | 3 | 53 | 29 | 16 | 1 | 1 | 22 | 9 |
| M5 | 6 | 2 | 6 | 11 | 42 | 277 | 2 | 75 | 17 | 7 | 1 | 1 | 20 | 8 |
| C1 | 6 | 3 | 5 | 12 | 44 | 23 | 1 | 43 | 1 | 48 | 7 | 0 | 22 | 10 |
| M1 | 9 | 6 | 5 | 11 | 37 | 300 | 5 | 75 | 17 | 6 | 1 | 0 | 20 | 9 |
| M2 | 9 | 7 | 5 | 10 | 34 | 213 | 4 | 57 | 19 | 21 | 2 | 0 | 20 | 10 |
| M3 | 9 | 8 | 5 | 11 | 38 | 252 | 6 | 82 | 5 | 10 | 2 | 1 | 20 | 10 |
| M4 | 9 | 8 | 6 | 12 | 41 | 332 | 4 | 51 | 1 | 46 | 1 | 1 | 22 | 9 |
| M5 | 9 | 5 | 5 | 11 | 38 | 329 | 4 | 84 | 7 | 9 | 1 | 0 | 21 | 8 |
| M1 | 12 | 7 | 5 | 11 | 38 | 288 | 6 | 84 | 9 | 3 | 3 | 1 | 20 | 9 |
| M2 | 12 | 7 | 5 | 10 | 32 | 229 | 5 | 79 | 6 | 13 | 2 | 1 | 20 | 11 |
| M3 | 12 | 7 | 5 | 11 | 36 | 282 | 6 | 84 | 5 | 7 | 3 | 1 | 20 | 10 |
| M4 | 12 | 8 | 4 | 12 | 40 | 257 | 3 | 39 | 1 | 60 | 2 | 1 | 30 | 9 |
| M5 | 12 | 4 | 5 | 10 | 35 | 351 | 3 | 87 | 5 | 6 | 2 | 1 | 21 | 8 |
| M1 | 15 | 7 | 5 | 11 | 40 | 292 | 5 | 80 | 7 | 8 | 4 | 1 | 20 | 9 |
| M2 | 15 | 5 | 4 | 9 | 33 | 45 | 3 | 60 | 28 | 9 | 3 | 0 | 19 | 10 |
| M3 | 15 | 7 | 5 | 11 | 35 | 268 | 6 | 79 | 11 | 8 | 2 | 1 | 20 | 9 |
| M4 | 15 | 6 | 5 | 12 | 39 | 389 | 3 | 48 | 1 | 48 | 2 | 1 | 22 | 9 |
| M5 | 15 | 5 | 5 | 10 | 35 | 388 | 4 | 82 | 4 | 13 | 1 | 1 | 21 | 8 |
| M1 | 21 | 6 | 5 | 10 | 35 | 255 | 3 | 55 | 31 | 11 | 2 | 1 | 21 | 9 |
| M2 | 21 | 5 | 5 | 9 | 34 | 251 | 4 | 88 | 3 | 9 | 0 | 0 | 19 | 10 |
| M3 | 21 | 6 | 5 | 11 | 36 | 310 | 5 | 80 | 7 | 11 | 2 | 1 | 20 | 9 |
| M4 | 21 | 6 | 5 | 11 | 40 | 405 | 4 | 66 | 25 | 7 | 2 | 1 | 22 | 9 |
| M5 | 21 | 3 | 5 | 11 | 36 | 330 | 2 | 80 | 13 | 5 | 2 | 1 | 21 | 8 |
| M1 | 28 | 5 | 5 | 10 | 37 | 289 | 2 | 44 | 51 | 2 | 2 | 1 | 20 | 9 |
| M2 | 28 | 3 | 4 | 9 | 28 | 199 | 2 | 67 | 13 | 18 | 2 | 0 | 24 | 10 |
| M3 | 28 | 6 | 5 | 11 | 38 | 255 | 5 | 82 | 8 | 9 | 1 | 0 | 20 | 10 |
| M4 | 28 | 5 | 6 | 13 | 43 | 327 | 2 | 36 | 2 | 61 | 1 | 0 | 23 | 10 |
| M5 | 28 | 3 | 5 | 11 | 36 | 305 | 2 | 60 | 22 | 16 | 2 | 1 | 21 | 8 |

WBC, white blood cells; RBC, red blood cells; HgB, hemoglobin; HCT, hematocrit level; PLT, platelets; Lymph, lymphocytes; Mono., monocytes; Neutr., neutrophils; Eosin., eosinophils; Baso., basophils; MCH, mean corpuscular hemoglobin; MPV, mean platelet volume.
M1-M5 are designated identifications of treated NHPs, and C1 is an untreated control NHP.

TABLE S2
Hematology measurements from rEBOV-515 + rEBOV-442 cocktail-treated
or -untreated individual NHP that were challenged with BDBV. Related to FIG. 4

| | | WBC | | | | | | | Mono. | | | | | |
|---|---|---|---|---|---|---|---|---|---|---|---|---|---|---|
| | Day | per | RBC per | | | Platelets | Lymph | | per | | | | | |
| Animal | after | mm³ | mm³ | HgB | Hct | per mm³ | per mm³ | Lymph. | mm³ | Neutr. | Eosin. | Baso. | MCH | MPV |
| ID | challenge | (×10³) | (×10³) | (g/dL) | (%) | (×10³ | (×10³) | (%) | (×10³) | (%) | (%) | (%) | (pg/cell) | (fL/cell) |
| M1 | 0 | 13 | 7 | 14 | 47 | 316 | 8 | 58 | 0 | 30 | 9 | 2 | 21 | 9 |
| M2 | 0 | 7 | 6 | 12 | 41 | 279 | 4 | 59 | 0 | 31 | 6 | 2 | 20 | 8 |
| M3 | 0 | 15 | 6 | 13 | 43 | 366 | 9 | 59 | 0 | 34 | 5 | 2 | 22 | 9 |
| M4 | 0 | 11 | 6 | 13 | 43 | 439 | 4 | 39 | 0 | 53 | 6 | 2 | 21 | 8 |
| M5 | 0 | 9 | 7 | 13 | 44 | 297 | 5 | 58 | 0 | 32 | 7 | 2 | 20 | 9 |
| C1 | 0 | 10 | 5 | 12 | 41 | 277 | 7 | 64 | 0 | 30 | 4 | 1 | 22 | 9 |
| M1 | 3 | 13 | 6 | 13 | 43 | 373 | 8 | 62 | 0 | 32 | 4 | 2 | 21 | 9 |
| M2 | 3 | 7 | 6 | 12 | 40 | 309 | 4 | 51 | 0 | 45 | 2 | 1 | 20 | 9 |
| M3 | 3 | 11 | 6 | 13 | 45 | 200 | 7 | 66 | 0 | 26 | 5 | 1 | 21 | 10 |
| M4 | 3 | 14 | 6 | 12 | 42 | 417 | 3 | 25 | 0 | 67 | 6 | 2 | 20 | 9 |
| M5 | 3 | 9 | 7 | 13 | 44 | 294 | 4 | 45 | 0 | 51 | 3 | 1 | 19 | 9 |
| C1 | 3 | 13 | 5 | 11 | 39 | 299 | 7 | 49 | 0 | 47 | 2 | 1 | 21 | 10 |
| M1 | 6 | 10 | 6 | 13 | 46 | 151 | 2 | 21 | 0 | 75 | 3 | 1 | 21 | 9 |
| M2 | 6 | 6 | 5 | 11 | 36 | 238 | 2 | 38 | 0 | 57 | 2 | 1 | 20 | 8 |
| M3 | 6 | 10 | 6 | 13 | 41 | 322 | 5 | 49 | 0 | 48 | 2 | 1 | 22 | 9 |
| M4 | 6 | 10 | 6 | 12 | 44 | 161 | 3 | 31 | 0 | 67 | 1 | 0 | 19 | 9 |
| M5 | 6 | 12 | 6 | 13 | 42 | 168 | 2 | 16 | 0 | 80 | 3 | 1 | 20 | 9 |
| C1 | 6 | 13 | 5 | 12 | 38 | 196 | 2 | 16 | 0 | 82 | 1 | 0 | 23 | 10 |
| M1 | 9 | 15 | 6 | 14 | 47 | 169 | 5 | 34 | 0 | 60 | 4 | 1 | 22 | 10 |
| M2 | 9 | 9 | 5 | 11 | 38 | 195 | 4 | 45 | 0 | 48 | 2 | 1 | 21 | 9 |
| M3 | 9 | 10 | 5 | 12 | 40 | 395 | 7 | 69 | 1 | 17 | 5 | 2 | 23 | 9 |
| M4 | 9 | 11 | 6 | 12 | 42 | 234 | 4 | 37 | 0 | 57 | 2 | 1 | 21 | 11 |
| M5 | 9 | 12 | 6 | 12 | 41 | 108 | 6 | 51 | 0 | 46 | 1 | 0 | 20 | 11 |
| C1 | 9 | 11 | 5 | 12 | 4 | 176 | 2 | 22 | 0 | 76 | 1 | 0 | 23 | 12 |
| M1 | 12 | 19 | 5 | 12 | 41 | 347 | 14 | 74 | 1 | 15 | 3 | 1 | 22 | 10 |
| M2 | 12 | 9 | 5 | 11 | 38 | 302 | 5 | 57 | 0 | 34 | 6 | 2 | 21 | 8 |
| M3 | 12 | 14 | 5 | 12 | 40 | 640 | 10 | 69 | 0 | 27 | 1 | 1 | 23 | 9 |
| M4 | 12 | 26 | 5 | 11 | 39 | 322 | 14 | 55 | 0 | 42 | 1 | 0 | 21 | 10 |
| M5 | 12 | 17 | 5 | 11 | 35 | 282 | 11 | 65 | 2 | 20 | 3 | 1 | 21 | 10 |
| C1 | 12 | 26 | 5 | 12 | 39 | 113 | 5 | 17 | 0 | 79 | 3 | 0 | 23 | 12 |
| C1 | 14 | 23 | 5 | 12 | 40 | 110 | 4 | 17 | 0 | 80 | 2 | 0 | 23 | 11 |
| M1 | 15 | 16 | 5 | 12 | 41 | 442 | 10 | 59 | 0 | 35 | 3 | 2 | 23 | 9 |
| M2 | 15 | 9 | 5 | 10 | 37 | 304 | 5 | 50 | 0 | 46 | 3 | 1 | 20 | 8 |
| M3 | 15 | 18 | 5 | 12 | 39 | 543 | 10 | 54 | 0 | 40 | 4 | 2 | 23 | 8 |
| M4 | 15 | 15 | 5 | 10 | 37 | 477 | 9 | 58 | 0 | 40 | 1 | 1 | 21 | 9 |
| M5 | 15 | 14 | 5 | 11 | 38 | 402 | 6 | 44 | 0 | 51 | 3 | 2 | 21 | 9 |
| M1 | 21 | 16 | 6 | 13 | 45 | 355 | 7 | 48 | 0 | 39 | 10 | 2 | 23 | 9 |
| M2 | 21 | 9 | 5 | 11 | 38 | 330 | 4 | 49 | 0 | 44 | 5 | 2 | 22 | 9 |
| M3 | 21 | 15 | 5 | 11 | 39 | 689 | 7 | 46 | 0 | 50 | 2 | 2 | 21 | 9 |
| M4 | 21 | 16 | 6 | 13 | 43 | 503 | 9 | 58 | 0 | 31 | 8 | 2 | 23 | 9 |
| M5 | 21 | 10 | 5 | 11 | 38 | 434 | 6 | 57 | 0 | 34 | 7 | 2 | 22 | 9 |
| M1 | 28 | 16 | 6 | 14 | 47 | 248 | 10 | 65 | 0 | 24 | 8 | 2 | 23 | 9 |
| M2 | 28 | 9 | 5 | 11 | 39 | 304 | 5 | 61 | 0 | 28 | 8 | 2 | 21 | 8 |
| M3 | 28 | 14 | 5 | 12 | 40 | 441 | 9 | 66 | 0 | 28 | 4 | 2 | 24 | 9 |
| M4 | 28 | 13 | 5 | 11 | 39 | 430 | 7 | 52 | 0 | 41 | 4 | 2 | 21 | 9 |
| M5 | 28 | 11 | 6 | 12 | 41 | 237 | 8 | 69 | 0 | 18 | 12 | 2 | 21 | 10 |
| M1 | 35 | 15 | 6 | 14 | 46 | 329 | 10 | 65 | 0 | 27 | 7 | 2 | 23 | 9 |
| M2 | 35 | 10 | 5 | 11 | 38 | 297 | 6 | 64 | 0 | 29 | 5 | 2 | 22 | 8 |
| M3 | 35 | 11 | 5 | 12 | 39 | 349 | 8 | 70 | 0 | 26 | 3 | 1 | 23 | 9 |
| M4 | 35 | 14 | 5 | 11 | 38 | 341 | 6 | 46 | 0 | 51 | 1 | 1 | 21 | 8 |
| M5 | 35 | 12 | 5 | 11 | 39 | 199 | 7 | 61 | 0 | 27 | 10 | 2 | 21 | 10 |

Hematology measurement designations are as in Table S1.
M1-M5 are designated identifications of treated NHPs, and C1 is an untreated control NHP.

TABLE S3
Hematology measurements from rEBOV-515 + rEBOV-442 cocktail-treated
or -untreated individual NHP that were challenged with SUDV. Related to FIG. 4

| | | WBC | RBC | | | | | | | | | | | |
|---|---|---|---|---|---|---|---|---|---|---|---|---|---|---|
| | Day | per | per | | | Platelets | Lymph | | Mono. | | | | | |
| Animal | after | mm³ | mm³ | HgB | Hct | per mm³ | per mm³ | Lymph. | per mm³ | Neutr. | Eosin. | Baso. | MCH | MPV |
| ID | challenge | (×10³) | (×10³) | (g/dL) | (%) | (×10³ | (×10³) | (%) | (×10³) | (%) | (%) | (%) | (pg/cell) | (fL/cell) |
| C1 | 0 | 5 | 7 | 14 | 49 | 311 | 4 | 81 | 0 | 6 | 3 | 1 | 20 | 9 |
| M1 | 0 | 6 | 6 | 13 | 41 | 265 | 5 | 82 | 0 | 14 | 1 | 0 | 22 | 11 |
| M2 | 0 | 4 | 5 | 12 | 41 | 395 | 3 | 62 | 1 | 20 | 1 | 1 | 22 | 8 |
| M3 | 0 | 6 | 5 | 11 | 37 | 305 | 4 | 75 | 1 | 11 | 1 | 0 | 21 | 8 |
| M4 | 0 | 8 | 4 | 11 | 31 | 183 | 7 | 90 | 0 | 7 | 0 | 0 | 26 | 9 |
| M5 | 0 | 7 | 6 | 12 | 44 | 431 | 3 | 46 | 3 | 12 | 4 | 2 | 22 | 9 |
| C1 | 4 | 16 | 6 | 12 | 46 | 255 | 2 | 11 | 0 | 85 | 2 | 1 | 19 | 8 |
| M1 | 4 | 13 | 5 | 12 | 42 | 241 | 2 | 16 | 5 | 42 | 3 | 1 | 22 | 11 |
| M2 | 4 | 17 | 5 | 11 | 37 | 327 | 1 | 6 | 0 | 89 | 2 | 1 | 22 | 9 |
| M3 | 4 | 13 | 5 | 10 | 34 | 240 | 1 | 11 | 0 | 85 | 3 | 1 | 20 | 8 |
| M4 | 4 | 12 | 5 | 10 | 35 | 189 | 7 | 62 | 4 | 6 | 1 | 0 | 22 | 9 |
| M5 | 4 | 30 | 5 | 12 | 43 | 352 | 1 | 4 | 0 | 92 | 2 | 1 | 22 | 9 |
| C1 | 7 | 5 | 6 | 11 | 41 | 87 | 2 | 28 | 2 | 25 | 3 | 0 | 20 | 10 |
| M1 | 7 | 8 | 5 | 11 | 38 | 270 | 3 | 41 | 3 | 28 | 1 | 1 | 22 | 11 |
| M2 | 7 | 12 | 5 | 10 | 34 | 316 | 2 | 13 | 6 | 37 | 2 | 1 | 21 | 8 |
| M3 | 7 | 10 | 5 | 10 | 38 | 232 | 2 | 17 | 0 | 73 | 7 | 2 | 19 | 8 |
| M4 | 7 | 14 | 5 | 10 | 35 | 256 | 4 | 26 | 8 | 13 | 2 | 1 | 21 | 9 |
| M5 | 7 | 17 | 5 | 11 | 40 | 392 | 2 | 12 | 0 | 83 | 2 | 1 | 21 | 9 |
| C1 | 10 | 3 | 6 | 11 | 40 | 27 | 1 | 27 | 0 | 67 | 5 | 0 | 19 | 10 |
| M1 | 10 | 6 | 5 | 11 | 38 | 445 | 5 | 86 | 1 | 2 | 1 | 0 | 21 | 10 |
| M2 | 10 | 20 | 4 | 10 | 32 | 352 | 3 | 14 | 9 | 41 | 1 | 1 | 22 | 9 |
| M3 | 10 | 6 | 5 | 9 | 33 | 358 | 4 | 71 | 1 | 8 | 2 | 1 | 19 | 8 |
| M4 | 10 | 8 | 4 | 9 | 29 | 330 | 7 | 87 | 1 | 5 | 0 | 0 | 22 | 9 |
| M5 | 10 | 12 | 5 | 10 | 38 | 572 | 4 | 37 | 0 | 58 | 3 | 2 | 21 | 8 |
| M1 | 14 | 6 | 5 | 11 | 38 | 472 | 4 | 77 | 1 | 9 | 1 | 1 | 21 | 10 |
| M2 | 14 | 6 | 4 | 10 | 32 | 659 | 4 | 62 | 0 | 26 | 3 | 2 | 23 | 8 |
| M3 | 14 | 6 | 5 | 10 | 36 | 429 | 4 | 58 | 0 | 36 | 5 | 2 | 19 | 8 |
| M4 | 14 | 7 | 4 | 9 | 35 | 464 | 7 | 94 | 0 | 2 | 0 | 0 | 21 | 9 |
| M5 | 14 | 17 | 4 | 10 | 34 | 423 | 3 | 15 | 9 | 27 | 2 | 1 | 23 | 8 |
| M1 | 21 | 5 | 5 | 12 | 35 | 275 | 5 | 82 | 0 | 6 | 2 | 1 | 25 | 11 |
| M2 | 21 | 6 | 5 | 10 | 37 | 441 | 2 | 38 | 0 | 56 | 5 | 2 | 22 | 8 |
| M3 | 21 | 5 | 5 | 10 | 37 | 367 | 4 | 70 | 0 | 20 | 4 | 2 | 19 | 8 |
| M4 | 21 | 8 | 5 | 11 | 37 | 397 | 7 | 91 | 0 | 5 | 1 | 1 | 23 | 9 |
| M5 | 21 | 8 | 5 | 11 | 37 | 432 | 2 | 30 | 4 | 17 | 2 | 1 | 23 | 9 |
| M1 | 28 | 5 | 6 | 12 | 43 | 223 | 4 | 80 | 0 | 9 | 2 | 1 | 20 | 11 |
| M2 | 28 | 4 | 5 | 11 | 40 | 365 | 2 | 49 | 0 | 47 | 3 | 1 | 22 | 8 |
| M3 | 28 | 4 | 5 | 10 | 34 | 167 | 3 | 79 | 1 | 2 | 2 | 1 | 20 | 9 |
| M4 | 28 | 9 | 5 | 10 | 35 | 168 | 8 | 91 | 0 | 2 | 1 | 0 | 22 | 10 |
| M5 | 28 | 7 | 5 | 10 | 36 | 295 | 3 | 42 | 3 | 12 | 3 | 1 | 22 | 9 |

Hematology measurement designations are as in Table S1.
M1-M5 are designated identifications of treated NHPs, and C1 is an untreated control NHP.

TABLE S4
Blood biochemistry measurements from rEBOV-515 + rEBOV-442 cocktail-
treated or -untreated NHP that were challenged with EBOV. Related to FIG. 4

| | | | | | | | | | | | | C-reactive |
|---|---|---|---|---|---|---|---|---|---|---|---|---|
| Animal | Day after | Glucose | BUN | Creatinine | Albumin | TP | ALT | AST | ALP | GGT | AMY | protein |
| ID | challenge | (mg/dL) | (mg/dL) | (mg/dL) | (g/dL) | (g/dL) | (U/L) | (U/L) | (U/L) | (U/L) | (U/L) | (mg/dL) |
| M1 | 0 | 88 | 34 | 1 | 4 | 8 | 46 | 32 | 252 | 72 | 273 | <5.0 |
| M2 | 0 | 98 | 20 | 1 | 4 | 7 | 58 | 26 | 204 | 63 | 256 | <5.0 |
| M3 | 0 | 76 | 17 | 0 | 4 | 7 | 45 | 32 | 440 | 104 | 166 | <5.0 |
| M4 | 0 | 65 | 20 | 1 | 3 | 7 | 123 | 60 | 209 | 78 | 307 | <5.0 |
| M5 | 0 | 79 | 14 | 1 | 3 | 6 | 38 | 32 | 311 | 77 | 320 | <5.0 |
| C1 | 0 | 93 | 15 | 0 | 3 | 8 | 41 | 27 | 266 | 92 | 296 | <5.0 |
| M1 | 3 | 94 | 43 | 1 | 3 | 8 | 48 | 32 | 255 | 72 | 297 | <5.0 |
| M2 | 3 | 82 | 21 | 1 | 3 | 7 | 62 | 29 | 170 | 59 | 276 | <5.0 |
| M3 | 3 | 85 | 23 | 1 | 4 | 7 | 44 | 31 | 414 | 100 | 203 | <5.0 |
| M4 | 3 | 67 | 20 | 1 | 3 | 6 | 147 | 67 | 196 | 75 | 324 | <5.0 |
| M5 | 3 | 61 | 20 | 1 | 3 | 6 | 33 | 32 | 310 | 72 | 370 | 5.0 |
| C1 | 3 | 92 | 14 | 1 | 3 | 8 | 41 | 29 | 230 | 82 | 335 | 15.8 |
| M1 | 6 | 85 | 38 | 1 | 3 | 7 | 38 | 30 | 216 | 69 | 353 | <5.0 |
| M2 | 6 | 69 | 22 | 1 | 3 | 7 | 51 | 32 | 146 | 58 | 259 | <5.0 |
| M3 | 6 | 74 | 21 | 1 | 3 | 7 | 35 | 35 | 336 | 94 | 172 | <5.0 |
| M4 | 6 | 72 | 25 | 1 | 3 | 7 | 102 | 48 | 182 | 75 | 301 | <5.0 |
| M5 | 6 | 62 | 23 | 1 | 3 | 6 | 47 | 63 | 301 | 66 | 316 | <5.0 |
| C1 | 6 | 13 | 101 | 6 | 2 | 7 | 931 | 4170* | 1708 | 484 | 514 | 160.0 |
| M1 | 9 | 82 | 28 | 1 | 3 | 8 | 40 | 33 | 205 | 66 | 250 | <5.0 |
| M2 | 9 | 73 | 19 | 1 | 3 | 7 | 55 | 32 | 142 | 57 | 232 | <5.0 |
| M3 | 9 | 83 | 23 | 1 | 3 | 7 | 38 | 38 | 337 | 89 | 210 | <5.0 |
| M4 | 9 | 69 | 24 | 1 | 3 | 7 | 103 | 60 | 177 | 73 | 302 | <5.0 |
| M5 | 9 | 63 | 26 | 1 | 3 | 6 | 48 | 46 | 271 | 66 | 289 | <5.0 |
| M1 | 12 | 81 | 27 | 1 | 3 | 8 | 34 | 33 | 211 | 68 | 280 | <5.0 |
| M2 | 12 | 84 | 21 | 1 | 3 | 7 | 49 | 30 | 144 | 58 | 278 | <5.0 |
| M3 | 12 | 67 | 23 | 1 | 3 | 7 | 38 | 34 | 360 | 88 | 201 | <5.0 |
| M4 | 12 | 49 | 23 | 1 | 3 | 7 | 131 | 70 | 167 | 72 | 320 | <5.0 |
| M5 | 12 | 62 | 22 | 1 | 3 | 6 | 34 | 37 | 274 | 65 | 316 | <5.0 |
| M1 | 15 | 89 | 25 | 1 | 3 | 8 | 32 | 31 | 214 | 66 | 260 | 8.3 |
| M2 | 15 | 81 | 19 | 1 | 3 | 7 | 63 | 38 | 138 | 56 | 249 | <5.0 |
| M3 | 15 | 71 | 19 | 1 | 3 | 7 | 44 | 35 | 384 | 87 | 196 | <5.0 |
| M4 | 15 | 61 | 21 | 1 | 3 | 7 | 124 | 54 | 168 | 69 | 300 | <5.0 |
| M5 | 15 | 74 | 17 | 1 | 3 | 6 | 31 | 31 | 292 | 66 | 341 | <5.0 |
| M1 | 21 | 67 | 34 | 0 | 3 | 8 | 29 | 29 | 203 | 64 | 263 | <5.0 |
| M2 | 21 | 84 | 19 | 1 | 3 | 7 | 50 | 28 | 108 | 57 | 254 | <5.0 |
| M3 | 21 | 72 | 21 | 1 | 4 | 7 | 40 | 37 | 366 | 91 | 186 | <5.0 |
| M4 | 21 | 67 | 24 | 1 | 3 | 7 | 85 | 42 | 147 | 63 | 310 | <5.0 |
| M5 | 21 | 61 | 24 | 1 | 3 | 6 | 26 | 27 | 316 | 68 | 358 | <5.0 |
| M1 | 28 | 58 | 32 | 1 | 3 | 7 | 32 | 29 | 208 | 64 | 240 | 5.8 |
| M2 | 28 | 74 | 20 | 1 | 4 | 7 | 74 | 36 | 120 | 58 | 257 | <5.0 |
| M3 | 28 | 66 | 22 | 1 | 4 | 7 | 38 | 35 | 362 | 93 | 166 | <5.0 |
| M4 | 28 | 45 | 23 | 1 | 3 | 7 | 81 | 42 | 129 | 55 | 275 | <5.0 |
| M5 | 28 | 53 | 24 | 1 | 3 | 5 | 22 | 39 | 243 | 57 | 272 | <5.0 |

BUN, blood urea nitrogen; TP, total protein; ALT, alanine aminotransferase; AST, aspartate aminotransferase; ALP, alkaline phosphatase; GGT, gamma-glutamyl transpeptidase; AMY, amylase.
*Sample was assessed at 10-fold dilution, and the value from the measurement was multiplied by ten to generate the recorded value.
M1-M5 are designated identifications of treated NHPs, and C1 is an untreated control NHP.

TABLE S5
Blood biochemistry measurements from rEBOV-515 + rEBOV-442 cocktail-
treated or -untreated NHP that were challenged with BDBV. Related to FIG. 4

| | | | | | | | | | | | | C-reactive |
|---|---|---|---|---|---|---|---|---|---|---|---|---|
| Animal | Day after | Glucose | BUN | Creatinine | Albumin | TP | ALT | AST | ALP | GGT | AMY | protein |
| ID | challenge | (mg/dL) | (mg/dL) | (mg/dL) | (g/dL) | (g/dL) | (U/L) | (U/L) | (U/L) | (U/L) | (U/L) | (mg/dL) |
| M1 | 0 | 87 | 19 | 1 | 3 | 7 | 31 | 33 | 463 | 63 | 338 | <5.0 |
| M2 | 0 | 64 | 19 | 1 | 3 | 7 | 28 | 47 | 294 | 73 | 270 | 9 |
| M3 | 0 | 84 | 21 | 1 | 3 | 7 | 27 | 31 | 280 | 66 | 467 | 9 |
| M4 | 0 | 83 | 20 | 1 | 3 | 7 | 55 | 35 | 222 | 73 | 458 | <5.0 |
| M5 | 0 | 93 | 20 | 1 | 3 | 7 | 30 | 40 | 270 | 46 | 296 | 7 |
| C1 | 0 | 89 | 18 | 1 | 3 | 6 | 27 | 30 | 171 | 38 | 217 | <5.0 |
| M1 | 3 | 67 | 19 | 1 | 3 | 7 | 37 | 33 | 474 | 60 | 324 | <5.0 |
| M2 | 3 | 66 | 19 | 1 | 3 | 8 | 22 | 40 | 309 | 74 | 261 | 7 |
| M3 | 3 | 85 | 20 | 1 | 3 | 7 | 35 | 39 | 290 | 63 | 487 | 11 |
| M4 | 3 | 78 | 21 | 1 | 3 | 7 | 65 | 34 | 216 | 75 | 462 | <5.0 |
| M5 | 3 | 76 | 20 | 0 | 3 | 8 | 40 | 41 | 278 | 16 | 291 | 7 |
| C1 | 3 | 90 | 17 | 1 | 3 | 6 | 33 | 29 | 166 | 38 | 207 | <5.0 |
| M1 | 6 | 87 | 20 | 1 | 3 | 7 | 33 | 55 | 387 | 53 | 265 | 123 |
| M2 | 6 | 71 | 18 | 1 | 3 | 7 | 22 | 44 | 286 | 66 | 253 | 7 |
| M3 | 6 | 82 | 18 | 1 | 3 | 7 | 32 | 29 | 247 | 54 | 336 | 106 |
| M4 | 6 | 100 | 15 | 1 | 3 | 7 | 29 | 52 | 171 | 34 | 178 | 101 |
| M5 | 6 | 83 | 20 | 1 | 3 | 7 | 30 | 44 | 216 | 37 | 240 | 136 |
| C1 | 6 | 93 | 17 | 1 | 2 | 7 | 57 | 106 | 294 | 81 | 336 | 164 |
| M1 | 9 | 76 | 22 | 1 | 3 | 8 | 168 | 242 | 403 | 73 | 232 | 25 |
| M2 | 9 | 66 | 26 | 1 | 3 | 7 | 26 | 52 | 312 | 66 | 267 | 14 |
| M3 | 9 | 82 | 23 | 1 | 3 | 7 | 37 | 29 | 218 | 55 | 456 | 26 |
| M4 | 9 | 80 | 26 | 1 | 2 | 7 | 147 | 66 | 421 | 120 | 303 | 33 |
| M5 | 9 | 78 | 22 | 1 | 3 | 7 | 116 | 94 | 273 | 76 | 196 | 39 |
| C1 | 9 | 81 | 33 | 1 | 2 | 6 | 55 | 114 | 471 | 186 | 150 | 23 |
| M1 | 12 | 74 | 17 | 1 | 3 | 7 | 82 | 42 | 297 | 63 | 344 | 7 |
| M2 | 12 | 66 | 19 | 1 | 3 | 7 | 29 | 46 | 379 | 83 | 287 | 10 |
| M3 | 12 | 90 | 19 | 1 | 3 | 7 | 31 | 30 | 143 | 61 | 528 | 11 |
| M4 | 12 | 81 | 22 | 1 | 2 | 7 | 56 | 38 | 315 | 98 | 339 | 7 |
| M5 | 12 | 72 | 23 | 1 | 3 | 7 | 51 | 47 | 202 | 52 | 356 | 9 |
| C1 | 12 | 107 | >180 | 6 | 2 | 6 | 66 | 164 | 566 | 338 | 305 | <5.0 |
| C1 | 14 | 190 | 380* | 12 | 1 | 5 | 85 | 227 | 357 | 198 | 63 | <5.0 |
| M1 | 15 | 66 | 20 | 1 | 3 | 7 | 49 | 34 | 340 | 64 | 337 | 6 |
| M2 | 15 | 55 | 20 | 1 | 3 | 7 | 34 | 50 | 309 | 73 | 245 | 10 |
| M3 | 15 | 75 | 21 | 1 | 3 | 7 | 35 | 47 | 249 | 61 | 398 | 22 |
| M4 | 15 | 72 | 25 | 1 | 3 | 7 | 68 | 47 | 309 | 108 | 414 | 6 |
| M5 | 15 | 64 | 21 | 1 | 3 | 7 | 41 | 48 | 205 | 49 | 329 | 9 |
| M1 | 21 | 76 | 17 | 0 | 3 | 7 | 44 | 35 | 418 | 56 | 340 | 5 |
| M2 | 21 | 70 | 18 | 1 | 3 | 7 | 28 | 43 | 273 | 74 | 259 | 8 |
| M3 | 21 | 75 | 24 | 1 | 3 | 7 | 56 | 32 | 245 | 97 | 414 | <5.0 |
| M4 | 21 | 83 | 20 | 1 | 3 | 7 | 25 | 29 | 272 | 63 | 415 | 9 |
| M5 | 21 | 77 | 20 | 1 | 3 | 8 | 27 | 41 | 207 | 42 | 281 | 7 |
| M1 | 28 | 85 | 22 | 1 | 3 | 7 | 28 | 37 | 271 | 64 | 418 | 8 |
| M2 | 28 | 66 | 19 | 1 | 3 | 7 | 23 | 45 | 272 | 69 | 259 | 6 |
| M3 | 28 | 85 | 20 | 1 | 3 | 7 | 39 | 33 | 379 | 52 | 364 | <5.0 |
| M4 | 28 | 71 | 24 | 1 | 3 | 7 | 56 | 40 | 214 | 75 | 431 | <5.0 |
| M5 | 28 | 73 | 21 | 1 | 3 | 7 | 29 | 41 | 202 | 40 | 311 | 7 |
| M1 | 35 | 79 | 19 | 1 | 3 | 7 | 53 | 34 | 395 | 53 | 365 | <5.0 |
| M2 | 35 | 63 | 17 | 1 | 3 | 7 | 22 | 41 | 275 | 66 | 288 | 8 |
| M3 | 35 | 73 | 21 | 1 | 3 | 6 | 33 | 31 | 283 | 60 | 396 | 11 |
| M4 | 35 | 78 | 25 | 1 | 3 | 6 | 61 | 43 | 208 | 64 | 40 | 5 |
| M5 | 35 | 66 | 21 | 1 | 3 | 6 | 35 | 36 | 231 | 42 | 369 | 6 |

Blood chemistry measurement designations are as in Table S4.
*Sample was assessed at 5-fold dilution, and the value from the measurement was multiplied by five to generate the recorded value.
M1-M5 are designated identifications of treated NHPs, and C1 is an untreated control NHP.

TABLE S6
Blood biochemistry measurements from rEBOV-515 + rEBOV-442 cocktail-
treated or -untreated NHP that were challenged with SUDV. Related to FIG. 4

| | | | | | | | | | | | | C-reactive |
|---|---|---|---|---|---|---|---|---|---|---|---|---|
| Animal | Day after | Glucose | BUN | Creatinine | Albumin | TP | ALT | AST | ALP | GGT | AMY | protein |
| ID | challenge | (mg/dL) | (mg/dL) | (mg/dL) | (g/dL) | (g/dL) | (U/L) | (U/L) | (U/L) | (U/L) | (U/L) | (mg/dL) |
| C1 | 0 | 97 | 23 | 1 | 3 | 7 | 119 | 43 | 388 | 82 | 397 | <5.0 |
| M1 | 0 | 115 | 27 | 1 | 4 | 7 | 64 | 35 | 262 | 81 | 345 | <5.0 |
| M2 | 0 | 82 | 15 | 1 | 3 | 7 | 84 | 33 | 247 | 73 | 286 | <5.0 |
| M3 | 0 | 83 | 19 | 1 | 3 | 7 | 67 | 33 | 217 | 59 | 311 | <5.0 |
| M4 | 0 | 89 | 28 | 1 | 3 | 7 | 49 | 35 | 213 | 67 | 349 | <5.0 |
| M5 | 0 | 80 | 23 | 1 | 3 | 7 | 34 | 32 | 258 | 61 | 344 | <5.0 |
| C1 | 4 | 89 | 20 | 1 | 3 | 7 | 82 | 38 | 322 | 71 | 211 | 125 |
| M1 | 4 | 88 | 17 | 1 | 3 | 6 | 61 | 31 | 202 | 55 | 228 | 109 |
| M2 | 4 | 93 | 14 | 0 | 3 | 7 | 76 | 34 | 208 | 65 | 220 | 103 |
| M3 | 4 | 88 | 19 | 1 | 3 | 7 | 50 | 33 | 226 | 67 | 228 | 129 |
| M4 | 4 | 81 | 24 | 1 | 3 | 6 | 50 | 34 | 169 | 60 | 393 | <5.0 |
| M5 | 4 | 86 | 17 | 1 | 3 | 7 | 44 | 34 | 241 | 54 | 226 | 139 |
| C1 | 7 | 73 | 24 | 1 | 2 | 6 | 298 | 379 | 990 | 123 | 162 | 121 |
| M1 | 7 | 63 | 20 | 1 | 3 | 7 | 73 | 64 | 230 | 50 | 269 | 6 |
| M2 | 7 | 80 | 13 | 1 | 3 | 6 | 59 | 31 | 199 | 55 | 180 | 29 |
| M3 | 7 | 75 | 23 | 1 | 3 | 7 | 53 | 34 | 248 | 64 | 227 | 26 |
| M4 | 7 | 81 | 29 | 1 | 3 | 7 | 42 | 34 | 172 | 56 | 293 | 21 |
| M5 | 7 | 82 | 21 | 1 | 3 | 7 | 87 | 39 | 391 | 50 | 219 | 90 |
| C1 | 10 | 59 | 36 | 1 | 2 | 6 | 159 | 478 | 1364 | 165 | 124 | 140 |
| M1 | 10 | 56 | 17 | 1 | 3 | 7 | 53 | 32 | 208 | 46 | 269 | <5.0 |
| M2 | 10 | 108 | 16 | 1 | 2 | 6 | 28 | 30 | 308 | 51 | 230 | 168 |
| M3 | 10 | 71 | 19 | 1 | 3 | 7 | 37 | 30 | 225 | 61 | 280 | <5.0 |
| M4 | 10 | 78 | 23 | 0 | 3 | 6 | 43 | 35 | 174 | 49 | 291 | 17 |
| M5 | 10 | 72 | 21 | <0.2 | 2 | 7 | 55 | 33 | 290 | 49 | 276 | <5.0 |
| M1 | 14 | 67 | 18 | 0 | 3 | 7 | 54 | 29 | 228 | 53 | 287 | <5.0 |
| M2 | 14 | 55 | 13 | 0 | 2 | 8 | 33 | 30 | 310 | 61 | 198 | 6 |
| M3 | 14 | 69 | 20 | 1 | 3 | 7 | 31 | 32 | 231 | 66 | 273 | <5.0 |
| M4 | 14 | 74 | 23 | 1 | 3 | 7 | 34 | 29 | 174 | 52 | 321 | <5.0 |
| M5 | 14 | 73 | 20 | 0 | 3 | 7 | 38 | 34 | 248 | 51 | 285 | <5.0 |
| M1 | 21 | 61 | 19 | 1 | 3 | 7 | 66 | 36 | 231 | 56 | 300 | <5.0 |
| M2 | 21 | 76 | 15 | 1 | 3 | 7 | 38 | 33 | 233 | 66 | 265 | <5.0 |
| M3 | 21 | 79 | 24 | 1 | 3 | 7 | 32 | 27 | 230 | 68 | 313 | <5.0 |
| M4 | 21 | 72 | 26 | 1 | 3 | 7 | 38 | 31 | 160 | 54 | 356 | <5.0 |
| M5 | 21 | 76 | 24 | 0 | 3 | 7 | 36 | 32 | 234 | 54 | 313 | <5.0 |
| M1 | 28 | 59 | 20 | 1 | 3 | 7 | 76 | 34 | 235 | 61 | 294 | <5.0 |
| M2 | 28 | 70 | 16 | 1 | 3 | 7 | 41 | 27 | 213 | 57 | 266 | <5.0 |
| M3 | 28 | 70 | 23 | 1 | 4 | 7 | 37 | 31 | 212 | 67 | 296 | <5.0 |
| M4 | 28 | 66 | 29 | 1 | 3 | 7 | 36 | 36 | 169 | 56 | 308 | <5.0 |
| M5 | 28 | 69 | 23 | 1 | 3 | 7 | 28 | 29 | 179 | 50 | 275 | <5.0 |

Hematology measurement designations are as in Table S1.
M1-M5 are designated identifications of treated NHPs, and C1 is an untreated control NHP.

TABLE S7
Cryo-EM data collection and statistics of EBOV GP ΔMucΔTM:rEBOV-
515:rEBOV-442 Fab complex. Related to FIG. 6

| | EBOV GPΔMucΔTM:rEBOV-442 + |
|---|---|
| Map | rEBOV-515 |
| Sample vitrification | — |
| Concentration | 3 mg/mL |
| Grid type | 1.2/1.3 Quantifoil |
| Detergent | 0.01% (w/v) F-OM |
| Blot time | 6 s |
| Data collection | 19aug23b |
| Microscope | Titan Krios |
| Voltage (kV) | 300 |
| Detector | K2 Summit |
| Recording mode | Counting |
| Magnification | 48,543 |
| Movie micrograph pixel size (Å) | 1.03 |
| Dose rate (e−/[(camera pixel)*s]) | 6.43 |
| Number of frames per movie | 42 |
| Frame exposure time (ms) | 250 |
| Movie micrograph exposure time (s) | 10.5 |
| Total dose (e−/Å²) | 50 |
| Defocus range (μm) | −1 to −3.5 |
| EM data processing | — |
| Software used | Relion 3.1b |
| Number of movie micrographs | 1,020 |
| Number of particles in map | 21,285 |
| Symmetry | C3 |
| Map resolution (FSC 0.143; Å) | 3.3 |
| Map sharpening B-factor (Å²) | −92 |
| Structure building and validation | — |
| Number of atoms in deposited model | 18,783 |
| GP1 | 5,541 |
| GP2 | 2,373 |
| rEBOV-442 | 5,571 |
| rEBOV-515 | 5,298 |
| Glycans | 534 |
| MolProbity score | 1.02 |
| Clashscore | 1.84 |
| EMRinger score | 3.62 |
| RMSD from ideal | — |
| Bond length (Å) | 0.021 |
| Bond angle (degrees) | 1.8 |
| Ramachandran plot | — |
| Favored (%) | 97.65 |
| Allowed (%) | 1.69 |
| Outliers (%) | 0.65 |
| Average B-factor | 128 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
U.S. Pat. No. 6,485,982
“Antibodies: A Laboratory Manual,” Cold Spring Harbor Press, Cold Spring Harbor, N Y, 1988.
Abbondanzo et al., Am. J. Pediatr. Hematol. Oncol. 12(4): 480-489, 1990.
Allred et al., Arch. Surg. 125(1), 107-113: 1990.
Atherton et al., Biol, of Reproduction 32: 155-171, 1985.
Barzon et al., Euro Surveill. 11: 21(32), 2016.
Beltramello et al., Cell Host Microbe 8: 271-283, 2010.
Brown et al., J. Immunol. Meth. 130(1): 111-121, 1990.
Campbell, In: Monoclonal Antibody Technology, Laboratory Techniques in Biochemistry and Molecular Biology, Vol. 13, Burden and Von Knippenberg, Eds, pp. 75-83, Amsterdam, Elsevier, 1984.
Capaldi et al., Biochem. Biophys. Res. Comm. 74(2):4 25-433, 1977.
De Jager et al., Semin. Nucl. Med. 23(2): 165-179, 1993.
Dholakia et al., J. Biol. Chem. 264: 20638-20642, 1989.
Diamond et al., J. Virol. 77: 2578-2586, 2003.
Doolittle and Ben-Zeev, Methods Mol. Biol. 109: 215-237, 1999.
Duffy et al., N. Engl. J. Med. 360: 2536-2543, 2009.
Elder et al. Infections, infertility and assisted reproduction. Part II: Infections in reproductive medicine & Part III: Infections and the assisted reproductive laboratory. Cambridge UK: Cambridge University Press; 2005.
Gefter et al., Somatic Cell Genet. 3: 231-236, 1977.
Gornet et al., Semin Reprod Med. 34(5): 285-292, 2016.
Gulbis and Galand, Hum. Pathol. 24(12): 1271-1285, 1993.
Halfon et al., PLOS ONE 5(5): e10569, 2010
Hessell et al., Nature 449: 101-104, 2007.
Khatoon et al., Ann, of Neurology 26: 210-219, 1989.
King et al., J. Biol. Chem. 269: 10210-10218, 1989.
Kohler and Milstein, Eur. J. Immunol. 6: 511-519, 1976.
Kohler and Milstein, Nature 256: 495-497, 1975.
Kyte and Doolittle, J. Mol. Biol. 157(1): 105-132, 1982.
Mansuy et al., Lancet Infect Dis. 16(10): 1106-7, 2016.
Nakamura et al., In: Enzyme Immunoassays: Heterogeneous and Homogeneous Systems, Chapter 27, 1987.
O'Shannessy et al., J. Immun. Meth. 99: 153-161, 1987.
Persic et al., Gene 187: 1, 1997
Potter and Haley, Meth. Enzymol. 91: 613-633, 1983.
Purpura et al., Lancet Infect Dis. 16(10): 1107-8, 2016.
Remington's Pharmaceutical Sciences, 15th Ed., 3: 624-652, 1990.
Tang et al., J. Biol. Chem., 271: 28324-28330, 1996.
Wawrzynczak & Thorpe, In: Immunoconjugates, Antibody Conuugates In Radioimaging And Therapy Of Cancer, Vogel (Ed.), NY, Oxford University Press, 28, 1987.
Yu et al., J Immunol Methods 336: 142-151, 2008.
Agirre et al. (2015). Nat. Struct. Mol. Biol. 22, 833-834.
Barad et al. (2015). Nat. Methods 12, 943-946.
Baum et al. (2020). Science 369, 1014-1018.
Bennett et al. (2017). Curr. Top. Microbiol. Immunol 411, 171-193.
Biasini et al. (2014). Nucleic Acids Res. 42, W252-258.
Bornholdt et al. (2019). Cell Host Microbe 25, 49-58 e45.
Bornholdt et al. (2016). mBio 7, e02154-02115.
Bray et al. (1998). J. Infect. Dis. 178, 651-661.
Brown et al. (2018). J. Infect. Dis. 218, 555-562.
Carette et al. (2011). Nature 477, 340-343.
Chandran et al. (2005). Science 308, 1643-1645.
Chng et al. (2015). mAbs 7, 403-412.
Corti et al. (2016). Science 351, 1339-1342.
Cross et al. (2016). J. Infect. Dis. 214, 565-569.
Davis et al. (2019). Cell 177, 1566-1582 e1517.
Diallo et al. (2016). Clin. Infect. Dis. 63, 1353-1356.
DiMaio et al. (2015). Nat. Methods 12, 361-365.
Domi et al. (2018). Sci. Rep. 8, 864.
Emsley et al. (2010). Features and development of Coot. Acta Crystallogr. D Biol. Crystallogr. 66, 486-501.
FDA (2020a). FDA Approves First Treatment for Ebola Virus. world-wide-web at fdagov/news-events/press-announcements/fda-approves-first-treatment-ebola-virus (accessed 21 Mar. 2021).
FDA (2020b). FDA Approves Treatment for Ebola Virus. world-wide-web fdagov/drugs/drug-safety-and-availability/fda-approves-treatment-ebola-virus (accessed 21 Mar. 2021).
Feldmann et al. (2020). New Engl. J. Med. 382, 1832-1842.
Fibriansah et al. (2015). Science 349, 88-91.
Garbutt et al. (2004). J. Virol. 78, 5458-5465.
Gaudinski et al. (2019). Lancet 393, 889-898.
Geisbert et al. (2008). J. Virol. 82, 5664-5668.
Geisbert et al. (2015). J. Infect. Dis. 212, S91-S97.
Gilchuk et al. (2020a). Nat. Biomed. Eng. 4, 1030-1043.
Gilchuk et al. (2018a). Immunity 49, 363-374 e310.
Gilchuk, et al. (2018b). J. Infect. Dis. 218, S565-S573.
Gilchuk et al. (2020b). Immunity 52, 388-403 e312.
Goldstein et al. (2019). Int. J. Infect. Dis. 79, 4-5.
Greaney, et al. (2021). Cell Host Microbe 29, 44-57 e9.
Herbert et al. (2020). Pros. Natl. Acad. Sci. U.S.A. 117, 3768-3778.
Ianevski et al. (2020). Nucleic Acids Res. 48, W488-W493.
Ilinykh, et al. (2016). J. Virol. 90, 3890-3901.
Iversen et al. (2020). Lancet Infect. Dis. 20, E231-E237.
Jubb et al. (2017). J. Mol. Biol. 429, 365-371.
King et al. (2019). Curr. Opin. Virol. 34, 140-148.
King et al. (2018). Curr. Opin. Immunol. 53, 196-202.
Kugelman et al. (2015). Cell Rep. 12, 2111-2120.
Kuhn et al. (2019). Nat. Rev. Microbiol. 17, 261-263.
Kuzmina et al. (2018). Cell Rep. 24, 1802-1815 e1805.
Laskowski, R. A., and Swindells, M. B. (2011). J. Chem. Inf. Model. 51, 2778-2786.
Lee et al. (2008). Nature 454, 177-182.
Lee, J. E., and Saphire, E. O. (2009). Curr. Opin. Struc. Biol. 19, 408-417.
Levine, M. M. (2019). New Engl. J. Med. 381, 2365-2366.
Liebschner, et al. (2019). Acta Crystallogr. D Biol. Crystallogr. 75, 861-877.
Lutteke, T., and von der Lieth, C. W. (2004). BMC Bioinformatics 5, 69.
Mbala-Kingebeni et al. (2019). Lancet Infect. Dis. 19, 648-657.
Mire et al. (2013). PLOS Negl. Trop. Dis. 7, e2600.
Misasi, J., and Sullivan, N.J. (2021). Immunity 54, 412-436.
Mulangu et al. (2019). New Engl. J. Med. 381, 2293-2303.
Murin et al. (2021). Cell Rep. (in press).
Orlandi et al. (2016). J. Immunol. Methods 433, 51-58.
Pascal et al. (2018). J. Infect. Dis. 218, S612-S626.
Pettersen et al. (2004). J. Comput. Chem. 25, 1605-1612.
Punjani et al. (2017). Nat. Methods 14, 290-296.
Qiu et al. (2014). Nature 514, 47-53.
Saphire et al. (2018). Cell 174, 938-952 e913.
Sivapalasingam, et al. (2018). Lancet Infect. Dis. 18, 884-893.
Starr et al. (2021). doi: 10.1101/2021.02.17.431683.
Subtil, et al. (2017). Clin. Infect. Dis. 64, 1788-1790.
Thi et al. (2016). Nat. Microbiol. 1, 16142.
Towner et al. (2005). Virology 332, 20-27.
Towner et al. (2008). PLOS Pathog. 4, e1000212.
Varke et al. (2015). New Engl. J. Med. 372, 2423-2427. virological.org (2021). Guinea 2021 EBOV outbreak. virologicalorg/t/guinea-2021-ebov-outbreak/643 (accessed 21 Mar. 2021).
Wang et al. (2021). Nature. doi.org/10.1038/s41586-021-03398-2
Wec et al. (2019). Cell Host Microbe 25, 39-48 e35.
Wec et al. (2017). Cell 169, 878-890 e815.
West et al. (2019). Nat. Struct. Mol. Biol. 26, 204-212.
WHO (2021). Ebola virus disease. world-wide-web at whoint/en/news-room/fact-sheets/detail/ebola-virus-disease (accessed 21 Mar. 2021)
Yewdell et al. (1979). Nature 279, 246-248.
Zheng et al. (2017). Nat. Methods 14, 331-332.
Zivanov et al. (2018). Elife 7.

1. A method of treating a subject infected with ebola virus, or reducing the likelihood of infection of a subject at risk of contracting ebola virus, comprising delivering to said subject at least first and second structurally distinct antibodies or antibody fragments, wherein said first antibody or antibody fragment has heavy and light chain CDR1-3 sequences of SEQ ID NOS: 5-7 and 8-10, respectively, wherein said second antibody or antibody fragment has heavy and light chain CDR1-3 sequences of SEQ ID NOS: 15-17 and 18-20, respectively.

2.-4. (canceled)

5. The method of claim 1, wherein said first antibody or antibody fragment comprises heavy and light chain sequences of SEQ ID NOS: 3 and 4, respectively, wherein said second antibody or antibody fragment comprises heavy and light chain sequences of SEQ ID NOS: 13 and 14, respectively.

6. The method of claim 1, wherein said first antibody or antibody fragment comprises heavy and light chain sequences having 70%, 80% 90% or 95% sequence identity to SEQ ID NOS: 3 and 4, respectively, wherein said second antibody or antibody fragment comprises heavy and light chain sequences having 70%, 80%, 90% or 95% sequence identity to SEQ ID NOS: 13 and 14, respectively.

7. (canceled)

8. The method of claim 1, wherein at least one of said antibody fragments is a recombinant scFv (single chain fragment variable) antibody, Fab fragment, F(ab′)₂ fragment, or Fv fragment.

9. The method of claim 1, wherein at least one of said antibodies is an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern.

10. The method of claim 1, wherein at least one of said antibodies is a chimeric antibody or a bispecific antibody.

11. The method of claim 1, wherein at least one of said antibodies or antibody fragments is administered prior to infection or after infection.

12. The method of claim 1, wherein said subject has been diagnosed with an ebola virus infection.

13. The method of claim 1, wherein delivering comprises antibody or antibody fragment administration, or genetic delivery with an RNA or DNA sequence or vector encoding the antibody or antibody fragment.

14. A composition comprising at least first and second structurally distinct antibodies or antibody fragments, wherein said first antibody or antibody fragment has heavy and light chain CDR1-3 sequences of SEQ ID NOS: 5-7 and 8-10, respectively, wherein said second antibody or antibody fragment has heavy and light chain CDR1-3 sequences of SEQ ID NOS: 15-17 and 18-20, respectively.

15. The composition of claim 14, wherein said first antibody or antibody fragment is encoded by heavy and light chain sequences of SEQ ID NOS: 1 and 2, respectively, wherein said second antibody or antibody fragment is encoded by heavy and light chain sequences of SEQ ID NOS: 11 and 12, respectively.

16. (canceled)

17. (canceled)

18. The composition of claim 14, wherein said first antibody or antibody fragment comprises heavy and light chain sequences of SEQ ID NOS: 3 and 4, respectively, wherein said second antibody or antibody fragment comprises heavy and light chain sequences of SEQ ID NOS: 13 and 14, respectively.

19. The composition of claim 14, wherein said first antibody or antibody fragment comprises heavy and light chain sequences having 70%, 80% 90% or 95% sequence identity to SEQ ID NOS: 3 and 4, respectively, wherein said second antibody or antibody fragment comprises heavy and light chain sequences having 70%, 80%, 90% or 95% sequence identity to SEQ ID NOS: 13 and 14, respectively.

20. (canceled)

21. The composition of claim 14, wherein at least one of said antibody fragments is a recombinant scFv (single chain fragment variable) antibody, Fab fragment, F(ab′)₂ fragment, or Fv fragment.

22. The composition of claim 14, wherein at least one of said antibodies is a chimeric antibody or is bispecific antibody.

23. The composition of claim 14, wherein at least one of said antibodies is an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern.

24. (canceled)

25. An engineered cell encoding at least first and second structurally distinct antibodies or antibody fragments, wherein said first antibody or antibody fragment has heavy and light chain CDR1-3 sequences of SEQ ID NOS: 5-7 and 8-10, respectively, wherein said second antibody or antibody fragment has heavy and light chain CDR1-3 sequences of SEQ ID NOS: 15-17 and 18-20, respectively.

26.-31. (canceled)

32. The engineered cell of claim 25, wherein at least one of said antibody fragments is a recombinant scFv (single chain fragment variable) antibody, Fab fragment, F(ab′)₂ fragment, or Fv fragment, or wherein at least one of said antibodies is a chimeric antibody or a bispecific antibody.

33. The engineered cell of claim 25, wherein at least one of said antibodies is an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern.

34-59. (canceled)

60. A method of protecting the health of a placenta and/or fetus of a pregnant a subject infected with or at risk of infection with ebola virus comprising delivering to said subject a composition according to claim 14.

61.-66. (canceled)


SEQUENCE LISTING (31 sequences)

SEQ ID NO: 1 - DNA, 408 bp, Artificial sequence
  Synthetic antibody
gaggtgcagttggtggagtctgggggaggcctcgtaaagccgggggggtcccttagactctcctgtacaggctctggaggcactggattcactttcaagtatgccgggatgagctgggtccgccaggctccagggaaggggccggagtggattggccgtattaaaagcaggattgatggtgggacaacagactacgctgcacccgtgaaagacagattcattgtctcaagagatgattcaagaaatacactctatctgcaaatgaacagcctgaagaccgaggacacagccgtctattattgtgccacaggatcgggaaaggggccctctgcgtcgttcggggagtcatactactactacgacttcattaacgtctggggcaaagggaccacggtcaccgtctcctca

SEQ ID NO: 2 - DNA, 324 bp, Artificial sequence
  Synthetic antibody
gaaagtgtgttgacgcagtctccaggcaccctgtctttgtctccaggggaaagagccaccctctcgtgcagggccagtcagagtattagcaggaagtacttagcctggtaccagcagaaacctggccaggctcccaggctcctcatctatggttcatccagcagggccactggcatcccagacagattcagtggcagtgggtctgggacagacttcactctcaccatcagcagactggagcctgaagattttgcagtgtattactgtcatcagtatgaaagctcaccttggacgttcggccaagggactaaggtggaaatcaaa

SEQ ID NO: 3 - protein, 136 aa, Artificial sequence
  Synthetic antibody
EVQLVESGGGLVKPGGSLRLSCTGSGGTGFTFKYAGMSWVRQAPGKGPEWIGRIKSRIDGGTTDYAAPVKDRFIVSRDDSRNTLYLQMNSLKTEDTAVYYCATGSGKGPSASFGESYYYYDFINVWGKGTTVTVSS

SEQ ID NO: 4 - protein, 108 aa, Artificial sequence
  Synthetic antibody
ESVLTQSPGTLSLSPGERATLSCRASQSISRKYLAWYQQKPGQAPRLLIYGSSSRATGIPDRFSGSGSGTDFTLTISRLEPEDFAVYYCHQYESSPWTFGQGTKVEIK

SEQ ID NO: 5 - protein, 8 aa, Artificial sequence
  Synthetic antibody
GFTFKYAG

SEQ ID NO: 6 - protein, 10 aa, Artificial sequence
  Synthetic antibody
IKSRIDGGTT

SEQ ID NO: 7 - protein, 24 aa, Artificial sequence
  Synthetic antibody
ATGSGKGPSASFGESYYYYDFINV

SEQ ID NO: 8 - protein, 7 aa, Artificial sequence
  Synthetic antibody
QSISRKY

SEQ ID NO: 9 - protein, 3 aa, Artificial sequence
  Synthetic antibody
GSS

SEQ ID NO: 10 - protein, 9 aa, Artificial sequence
  Synthetic antibody
HQYESSPWT

SEQ ID NO: 11 - DNA, 366 bp, Artificial sequence
  Synthetic antibody
caggtgcagctgcacgagtcgggcccaggactggtgaagccttcacagaccctgtccctcacctgcactgtctctggtgggtccatcaacagtgctggttactactggacctggatccgccagcacccggggaagggcctggagtggattgggtacatcgattatactgggaggacctactacaacccgtcccttgagagccgagtgatcatttcaatagacacgtctaagaaccacttctccctgagactgacctctgtgtctgccgcggacacggccgtgtattactgtgcgcgagaatcgtcgtgggtatccgagttagggcgtgacaactggggccagggaaccctggtcaccgtctcttca

SEQ ID NO: 12 - DNA, 321 bp, Artificial sequence
  Synthetic antibody
gaaatagtgatgacgcagtctccagccaccctgcctgtgtctccaggggaaggagccaccctctcctgcagggccagtcagagtgttttcaccaacttagcctggtaccagcaaaaacctggccaggctcccaggctcctcatctatgatgcatccaccagggccactggtatcccagccaggttcagtggcagtgggtctgggacagagttcactctcaccatcagcagcctgcagtctgaagactttgcagtttattattgtcagcagtataataactggcctcggacgtacggccaagggaccagggtggaagtcaaa

SEQ ID NO: 13 - protein, 122 aa, Artificial sequence
  Synthetic antibody
QVQLHESGPGLVKPSQTLSLTCTVSGGSINSAGYYWTWIRQHPGKGLEWIGYIDYTGRTYYNPSLESRVIISIDTSKNHFSLRLTSVSAADTAVYYCARESSWVSELGRDNWGQGTLVTVSS

SEQ ID NO: 14 - protein, 107 aa, Artificial sequence
  Synthetic antibody
EIVMTQSPATLPVSPGEGATLSCRASQSVFTNLAWYQQKPGQAPRLLIYDASTRATGIPARFSGSGSGTEFTLTISSLQSEDFAVYYCQQYNNWPRTYGQGTRVEVK

SEQ ID NO: 15 - protein, 10 aa, Artificial sequence
  Synthetic antibody
GGSINSAGYY

SEQ ID NO: 16 - protein, 7 aa, Artificial sequence
  Synthetic antibody
IDYTGRT

SEQ ID NO: 17 - protein, 14 aa, Artificial sequence
  Synthetic antibody
ARESSWVSELGRDN

SEQ ID NO: 18 - protein, 6 aa, Artificial sequence
  Synthetic antibody
QSVFTN

SEQ ID NO: 19 - protein, 3 aa, Artificial sequence
  Synthetic antibody
DAS

SEQ ID NO: 20 - protein, 9 aa, Artificial sequence
  Synthetic antibody
QQYNNWPRT

SEQ ID NO: 21 - DNA, 21 bp, Artificial sequence
  Synthetic primer
ccgtcaatcaaggagcgcctc

SEQ ID NO: 22 - DNA, 20 bp, Artificial sequence
  Synthetic primer
cgcaacctccacagtcgcct

SEQ ID NO: 23 - DNA, 21 bp, Artificial sequence
  Synthetic primer
catccaatcaaagacattgcg

SEQ ID NO: 24 - protein, 40 aa, Ebola virus
EWAENCYNLEIKKPDGSECLPAAPDGIRGFPRCRYVHKVS

SEQ ID NO: 25 - protein, 60 aa, Ebola virus
NAQPKCNPNLHYWTTQDEGAAIGLAWIPYFGPAAEGIYIEGLMHNQDGLICGLRQLANET

SEQ ID NO: 26 - protein, 63 aa, Ebola virus
NGVATDVPSAEWAENCYNLEIKKPDGSECLPAAPDGIRGFPRCRYVHKVSQLESRFTPQFLLQ

SEQ ID NO: 27 - protein, 60 aa, Ebola virus; identical to SEQ ID NO: 25

SEQ ID NO: 28 - protein, 60 aa, Ebola virus
LRSVGLNLEGNGVATDVPSATKRWGFRSGVPPKVVNYEAGEWAENCYNLEPRCRYVHKVS

SEQ ID NO: 29 - protein, 60 aa, Ebola virus; identical to SEQ ID NO: 25

SEQ ID NO: 30 - protein, 4 aa, Ebola virus
EQHN

SEQ ID NO: 31 - protein, 4 aa, Ebola virus
DRPH